(19) United States
(12) Patent Application Publication
(10) Pub. No.: US 2026/0248799 A1
(43) Pub. Date: Aug. 27, 2026

(54) COMBINATIONS OF RETINALDEHYDE DEHYDROGENASE 1 (RALDH1) INHIBITORS AND IMMUNOSTIMULATORS AND METHODS USING SAME

(71) Applicants: The Trustees of the University of Pennsylvania, Philadelphia, PA (US); Government of the United States of America, as represented by the Secretary, Department of Health an, Bethesda, MD (US)

(72) Inventors: Malay Haldar, Wynnewood, PA (US); Ganesha Rai Bantukallu, Rockville, MD (US); Shyh Ming Yang, Doylestown, PA (US); Natalia Julia Martinez, Rockville, MD (US); Anton Simeonov, Bethesda, MD (US); Alexey Vladimirovich Zakharov, Ijamsville, MD (US); Adam Scott Yasgar, Bethesda, MD (US)

(21) Appl. No.: 19/164,842

(22) PCT Filed: Mar. 15, 2024
(86) PCT No.: PCT/US2024/020160

§ 371 Date: Sep. 12, 2025

Related U.S. Application Data
(60) Provisional application No. 63/452,978, filed on Mar. 17, 2023.

(51) Int. Cl. A61K 31/52; A61K 35/17; A61K 40/11; A61K 40/31; A61P 35/00; C07K 16/28

(52) U.S. Cl. CPC A61K 31/52; A61K 35/17; A61K 40/11; A61K 40/31; A61P 35/00; C07K 16/2818

(57) ABSTRACT

The present disclosure relates in part to methods of treating a solid tumor in a subject in need thereof, the method comprising administering to the subject a retinaldehyde dehydrogenase 1 (RALDH1) inhibitor and an immunostimulator. The present disclosure further relates to pharmaceutical compositions comprising an RALDH1 inhibitor and an immunostimulator.

### CROSS REFERENCE TO RELATED APPLICATIONS

This application claims priority under 35 U.S.C. § 119(e) to U.S. Provisional Patent Application No. 63/452,978, filed Mar. 17, 2023, which application is incorporated herein by reference in its entirety.

### STATEMENT REGARDING FEDERALLY SPONSORED RESEARCH OR DEVELOPMENT

This invention was made with government support under W81XWH-21-1-0592 awarded by the Medical Research and Development Command, and CA234027 awarded by the National Institutes of Health. The government has certain rights in the invention.

### SEQUENCE LISTING

The XML file named “046483_7326W01_SequenceListing,” created on Mar. 14, 2024, comprising 6,408 bytes, is hereby incorporated by reference in its entirety.

### BACKGROUND OF THE INVENTION

Hepatocellular carcinoma (HCC) ranks as the sixth most common cancer and the third leading cause of cancer-related death globally. The presence of chronic liver disease is a significant risk factor. Early detection with surveillance in high-risk patients and better diagnostic approaches have improved outcomes, but advanced HCC continues to pose major management challenges. Depending on the grade and stage of HCC and the underlying liver function, surgical resection is the treatment of choice in most cases. Other options include liver transplantation, image-guided tumor ablation, tyrosine kinase inhibitors, and immune checkpoint blockers, the latter of which is often ineffective in patients. Despite the progress in management of early stage HCC, overall 5 year survival in advanced cases with distal metastases is about 2%.

There is thus a need in the art for methods and compositions for the treatment of solid tumors, including but not limited to HCC. The present disclosure addresses this need.

### BRIEF SUMMARY OF THE INVENTION

In one aspect, the disclosure provides a method of treating, preventing, and/or ameliorating a solid tumor in a subject in need thereof. In certain embodiments, the method comprises administering to the subject a pharmaceutically effective amount of:
(a) at least one immunostimulator; and
(b) a retinaldehyde dehydrogenase 1 (RALDH1) inhibitor, wherein the RALDH1 inhibitor is a compound of formula (I), or a salt, solvate, prodrug, stereoisomer, tautomer, or isotopologue thereof:

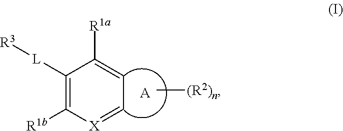

wherein:
R^{1a} is selected from the group consisting of optionally substituted C₂-C₈ heterocyclyl, optionally substituted phenyl, and optionally substituted C₅-C₈ cycloalkenyl,
   wherein each optional substituent in R^{1a} is independently selected from the group consisting of C₁-C₆ alkyl, C₃-C₆ cycloalkyl, C₁-C₆ alkoxy, optionally substituted phenyl, optionally substituted C₂-C₈ heterocyclyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b},
      wherein each optional substituent is optionally substituted with at least one substituent selected from the group consisting of C₁-C₃ alkyl, C₁-C₃ alkoxy, halogen, CN, and NO₂, and
   wherein two vicinal or geminal optional substituents in R^{1a} may combine with the atoms to which they are bound to form a C₂-C₈ heterocyclyl or C₃-C₈ cycloalkyl;
R^{1b} and R^{1c}, if present, are each independently selected from the group consisting of H, C₁-C₆ alkyl, C₁-C₆ alkoxy, halogen, OH, N(R^{a})(R^{b}), NO₂, and CN;
each occurrence of R² is independently selected from the group consisting of C₁-C₆ alkyl, C₁-C₆ alkoxy, C₁-C₃ haloalkoxy, C₁-C₆ hydroxyalkyl, halogen, NO₂, and CN;
R³ is selected from the group consisting of optionally substituted C₂-C₈ heterocyclyl, optionally substituted phenyl, N(R^{a})(optionally substituted C₃-C₈ cycloalkyl), and N(R^{a})(optionally substituted C₂-C₈ heterocyclyl),
   wherein each optional substituent in R³ is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b};
A is selected from the group consisting of optionally substituted C₆-C₁₀ aryl and optionally substituted C₂-C₈ heterocyclyl,
   wherein each optional substituent in A is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b};
L is selected from the group consisting of —CH₂—, —C(═O)—, and —S(═O)₂—;
X is selected from the group consisting of N and CR^{1c};
n is an integer selected from the group consisting of 0, 1, 2, 3, and 4; and
each occurrence of R^{a}, R^{b}, and R^{c} is independently selected from the group consisting of H, C₁-C₆ alkyl, C₂-C₆ alkenyl, C₂-C₆ alkynyl, C₃-C₈ cycloalkyl, and C₂-C₈ heterocyclyl.

In another aspect, the disclosure provides a method of treating, preventing, and/or ameliorating a solid tumor in a subject in need thereof. In certain embodiments, the method comprises administering to the subject a pharmaceutically effective amount of:
(a) at least one immunostimulator; and
(b) a retinaldehyde dehydrogenase 1 (RALDH1) inhibitor, wherein the RALDH1 inhibitor is a compound selected from the group consisting of:
   1-benzylindoline-2,3-dione;
   ethyl 2-((4-oxo-3-(3-(pyrrolidin-1-yl)propyl)-3,4-dihydrobenzo[4,5]thieno[3,2-d]pyrimidin-2-yl)thio)acetate;
   2-((2-(sec-butyl)-3-oxo-2,3-dihydroimidazo[1,2-c]quinazolin-5-yl)thio)-N-(o-tolyl)butanamide; and
   8-((4-(cyclopropanecarbonyl)piperazin-1-yl)methyl)-7-isopentyl-1,3-dimethyl-3,7-dihydro-1H-purine-2,6-dione;
   or a salt, solvate, prodrug, stereoisomer, tautomer, or isotopologue thereof.

In another aspect, the disclosure provides a pharmaceutical composition comprising:
(a) at least one immunostimulator;
(b) a pharmaceutically acceptable carrier; and
(c) a retinaldehyde dehydrogenase 1 (RALDH1) inhibitor, wherein the RALDH1 inhibitor is a compound of formula (I), or a salt, solvate, prodrug, stereoisomer, tautomer, or isotopologue thereof:

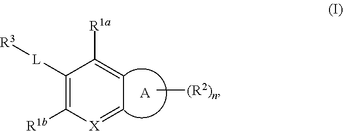

wherein:
R^{1a} is selected from the group consisting of optionally substituted C₂-C₈ heterocyclyl, optionally substituted phenyl, and optionally substituted C₅-C₈ cycloalkenyl,
   wherein each optional substituent in R^{1a} is independently selected from the group consisting of C₁-C₆ alkyl, C₃-C₆ cycloalkyl, C₁-C₆ alkoxy, optionally substituted phenyl, optionally substituted C₂-C₈ heterocyclyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b},
      wherein each optional substituent is optionally substituted with at least one substituent selected from the group consisting of C₁-C₃ alkyl, C₁-C₃ alkoxy, halogen, CN, and NO₂, and
   wherein two vicinal or geminal optional substituents in R^{1a} may combine with the atoms to which they are bound to form a C₂-C₈ heterocyclyl or C₃-C₈ cycloalkyl;
R^{1b} and R^{1c}, if present, are each independently selected from the group consisting of H, C₁-C₆ alkyl, C₁-C₆ alkoxy, halogen, OH, N(R^{a})(R^{b}), NO₂, and CN;
each occurrence of R² is independently selected from the group consisting of C₁-C₆ alkyl, C₁-C₆ alkoxy, C₁-C₃ haloalkoxy, C₁-C₆ hydroxyalkyl, halogen, NO₂, and CN;
R³ is selected from the group consisting of optionally substituted C₂-C₈ heterocyclyl, optionally substituted phenyl, N(R^{a})(optionally substituted C₃-C₈ cycloalkyl), and N(R^{a})(optionally substituted C₂-C₈ heterocyclyl),
   wherein each optional substituent in R³ is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b};
A is selected from the group consisting of optionally substituted C₆-C₁₀ aryl and optionally substituted C₂-C₈ heterocyclyl
   wherein each optional substituent in A is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b}.
L is selected from the group consisting of —CH₂—, —C(═O)— and —S(═O)₂—;
X is selected from the group consisting of N and CR^{1c};
n is an integer selected from the group consisting of 0, 1, 2, 3, and 4; and
each occurrence of R^{a}, R^{b}, and R^{c} is independently selected from the group consisting of H, C₁-C₆ alkyl, C₂-C₆ alkenyl, C₂-C₆ alkynyl, C₃-C₈ cycloalkyl, and C₂-C₈ heterocyclyl.

In another aspect, the disclosure provides a pharmaceutical composition comprising:
(a) at least one immunostimulator;
(b) a pharmaceutically acceptable carrier; and
(c) a retinaldehyde dehydrogenase 1 (RALDH1) inhibitor, wherein the RALDH1 inhibitor is a compound selected from the group consisting of:
   1-benzylindoline-2,3-dione,
   ethyl 2-((4-oxo-3-(3-(pyrrolidin-1-yl)propyl)-3,4-dihydrobenzo[4,5]thieno[3,2-d]pyrimidin-2-yl)thio)acetate;
   2-((2-(sec-butyl)-3-oxo-2,3-dihydroimidazo[1,2-c]quinazolin-5-yl)thio)-N-(o-tolyl)butanamide; and
   8-((4-(cyclopropanecarbonyl)piperazin-1-yl)methyl)-7-isopentyl-1,3-dimethyl-3,7-dihydro-1H-purine-2,6-dione;
   or a salt, solvate, prodrug, stereoisomer, tautomer, or isotopologue thereof.

### BRIEF DESCRIPTION OF THE FIGURES

The drawings illustrate generally, by way of example, but not by way of limitation, various embodiments of the present application.

FIG. 1 provides a schematic which illustrates a pathway by which tumor-derived retinoic acid (RA) promotes immune evasion.

FIG. 2 provides a schematic which illustrates the biosynthesis of RA from vitamin A (retinol) and the regulation of gene expression by RA in intratumoral monocytes.

FIG. 3 provides a bar graph showing the relative expression of RALDH1, RALDH2, and RALDH3 in human cell lines HT1080 (liposarcoma), SNU398 (hepatocellular carcinoma), and PLC (hepatoma) in vitro culture and in vivo xenograft (xeno) in NU/J mouse hosts.

FIG. 4 provides a bar graph representing gene expression of RALDH1, RALDH2, and RALDH3 in mouse HCC cell line (Hepa 1-6), normal liver, and fibrosarcoma cell line (B6-PRG) normalized to Hprt expression.

FIG. 5 provides a pharmacological profile of RALDH1 inhibitors C-86 and C-91. n=3; for each example the compound was formulated as a solution in 20% HP-β-CD in saline. Dosage; 2 mg/kg for intravenous (iv) and 10 mg/kg for oral (po) administration; plasma samples were measured for drug exposure by LC-MS/MS; CD-1 mouse or Sprague-Dawley rat were used; The maximum drug concentration (Cₘₐₓ) was observed at t=5 min, the first sample time point after iv administration.

FIG. 6 shows the RALDH activity of mouse (Hepa 1-6) and human (SNU398) HCC cell lines, and mouse fibrosarcoma (B6-PRG) cell lines as measured by fluorescence using the Aldered™ (aka Aldefluor) assay. Fluorescence reflects RALDH activity. Control (top row) shows baseline fluorescence while test (bottom row) shows fluorescence imparted by RALDH activity.

FIG. 7 shows that the RALDH activity, as detected by Aldered (Aldefluor) assay, was abrogated in SNU398 human HCC cells in vitro upon treatment with 100 nM of either C-86 or C-91, wherein DMSO served as a control treatment.

FIG. 8 shows the mean fluorescence intensity (MFI) of an Aldered assay wherein SNU398 cells were treated with various concentrations of C-86 or C-91 (1, 10, 100, and 1000 nM) and DMSO served as a control.

FIG. 9 provides a graph comparing the growth of SNU398 cells in the presence of C-86 or C-91 at various concentrations (1, 10, 100, and 1000 nM) as compared to a DMSO control, demonstrating that neither C-86 or C-91 significantly impact proliferation of SNU398 cells.

FIG. 10 provides plots showing the frequency of myeloid antigen presenting cells and T cells. Hepa 1-6 cells were transplanted into the flank of C57BL/6J mice. Tumors were dissociated into single-cell suspensions and subjected to flow cytometry with indicated markers.

FIG. 11A provides a schematic illustrating the experimental protocol utilized to demonstrate restoration by compound C-86 of activated T-cell proliferation for cells exposed to tumor conditioned media (CM); (a) monocytes harvested from mice or humans were exposed to vehicle (DMSO) or to compound C-86—in parallel, T-cells harvested from mice or humans were labeled (CFSE), then activated in vitro (anti-CD3/28) to evaluate their proliferation via dye dilution through flow cytometry; (b) monocyte-derived dendritic cells (MoDCs) and activated T-cells were combined in different ratios (1:0, 1:1, or 1:2), then exposed to the following conditions: vehicle (DMSO), compound C-86 alone, to SNU398 HCC cell conditioned media (SNU398-CM) or to CM in combination with C-86 (SNU398-CM+C-86). The proliferation was evaluated as described in (a) and compared to unstimulated T-cells.

FIG. 11B provides a bar graph showing the restoration by compound C-86 of activated T-cell proliferation for cells exposed to tumor conditioned media (CM). Addition of C-86 to MoDC and T cells exposed to SNU398-CM fully restored the proliferation rate as compared to vehicle.

FIGS. 12A-12C shows that intraperitoneal C-86 inhibits in vivo growth of hepatocellular carcinoma (HCC) cell line (Huh7) and shows synergy with BMS493. FIG. 12A: diagram showing mechanisms of inhibition of retinaldehyde (RA) synthesis in HCC cells and inhibition of RA mediated transcription in monocytes. FIGS. 12B-12C: results of intraperitoneal treatment of human HCC (Huh7) with C-86, BMS493, and C-86+BMS493, as compared to a control.

FIGS. 13A-13B show tumor volume (FIG. 13A) and tumor mass (FIG. 13B) with intraperitoneal administration of C-86.

FIGS. 14A-14B show that the in vivo effects of C-86 on tumors may be mediated by macrophages. FIG. 14A: macrophage depletion with liposomal chlodronate (CloLipo). FIG. 14B: results of treatment of human HCC (Huh7) with C-86, CloLipo, and C-86+CloLipo.

FIG. 15 provides pharmacokinetic (PK) data for compounds C-86 and C-97. ^{a}n=3. Formulation: 20% HP-β-CD in saline for IV and PO. ^{b}Dosage: 2 mg/kg for intravenous (IV) and 10 mg/kg for oral (PO) administration. ° CD-1 mouse was used. ^{d}Cₘₐₓ was observed at t=5 min, the first sampling time point for IV administration.

FIG. 16 shows that C-97 inhibits RALDH1 activity in human HCC cells (SNU398), as assessed in an Aldefluor assay; Controls: dimethylsulfoxide (DMSO); N,N-diethylaminobenzaldehyde (DEAB). DEAB is commonly used as a selective inhibitor of aldehyde dehydrogenase. WIN 18446 is a non-selective RALDH inhibitor.

FIGS. 17A-17B provides a comparison of orally administered C-86 and C-97 in HCC cell inhibition as assessed by tumor volume (FIG. 17A) and tumor mass (FIG. 17B).

FIGS. 18A-18I: HCCs overexpress RALDH1 to produce high levels of RA. FIG. 18A: Raldh1 mRNA levels (y-axis, RSEM, batch normalized) in different tumor types (x-axis) from TCGA data-based analyzed through the cBioPortal web interface. Data show higher expression of RALDH1 in liver cancer. AdC, adrenocortical carcinoma; BaC, bladder cancer; BrC, breast cancer; CvC, cervical cancer; CrC, colorectal cancer; EmC, endometrial cancer; EgC, esophagogastric cancer; GBM, glioblastoma; GL, glioma; HNC, head and neck cancer; HC, hepatobiliary cancer; LK, leukemia; MBN, mature B cell neoplasm; ML, melanoma; MNT. Miscellaneous neuroepithelial tumor; SGT, nonseminomatous germ cell tumor; OML, ocular melanoma; OET, ovarian epithelial tumor; PCT, pheochromocytoma; SMN, seminoma; TC, thyroid cancer. FIG. 18B: Raw sequencing counts for the 183 primary HCCs previously used to identify iCluster 1-3 molecular subtypes were downloaded and the expression levels of the three Raldh isozymes calculated. Raldh1 levels are significantly higher than the other two isozymes in all three molecular subtypes. FIG. 18C: Human tumors (header) were stained with anti-RALDH1 antibody. Arrows show tumor locations. Primary and metastatic HCC show strong RALDH1 staining whereas unrelated tumors, such as GIST and CRC show no staining. FIG. 18D: Transcript levels of Raldh1, Raldh2 and Raldh3 in multiple human HCC cell lines were measured by RT-qPCR. Raldh1 is the dominant isozyme expressed in all cell lines. FIG. 18E AldeRed assay on human HCC cell lines. “Control” shows AldeRed fluorescence with aldehyde dehydrogenase inhibitor DEAB whereas “Test” shows the same without the inhibitor, which distinguishes fluorescence through RALDH activity from the background. The histograms are representative of ≥3 experiments. Numbers denote the percentage of cells within indicated gate. All HCC cell lines show high RALDH activity in all cells (Huh7 and Hep3B), majority of cells (SNU398 and SNU449), or some cells (PLC). FIG. 18F: Transcript levels of Raldh1, Raldh2 and Raldh3 in multiple murine HCC cell lines were measured by RT-qPCR. Raldh1 is the dominant isozyme expressed in all cell lines. FIG. 18G: AldeRed assay in murine HCC line Hepa1-6 showing high RALDH activity in the majority of cells. FIG. 18H: The Raldh1 gene was deleted from Huh7 cells by using CRISPR/CAS9 (RALDH1-KO cell line). AldeRed assay performed on RALDH1-KO and the parental Huh7 cells show loss of AldeRed positivity in the KO. FIG. 18I: LC-MS-based measurement of ATRA in the indicated murine and human HCC cell lines (x-axis).

FIG. 19A: Transcript levels of Raldh1 in human tumor formalin fixed paraffin embedded (FFPE) samples measured by RT-qPCR. Raldh1 is higher in primary and metastatic HCC. HCC-M: Metastatic HCC; HCC-P: Primary HCC; GIST: Gastrointestinal Stromal Tumor; CrC: Colorectal Cancer; Lv: Normal Liver. One experiment with at least three replicates per sample type. One-way ANOVA. *p<0.05, **p<0.01, ***p<0.001. FIG. 19B: Raldh2 (top plot) and Raldh3 (bottom plot) transcript levels (Y-axis, RSEM, batch normalized) in different tumor types (X-axis) from the TCGA databased analyzed through the cBioPortal. FIG. 19C: Transcript levels of Raldh1, Raldh2 and Raldh3 in human HCC FFPE samples measured by RT-qPCR. Raldh1 is the dominant isozyme in the majority of specimens. One experiment with at least three replicates per sample type. One-way ANOVA. *p<0.05, **p<0.01, ***p<0.001. FIG. 19D: De-identified human tumors (archived formalin fixed and paraffin embedded) were sectioned and stained with anti-RALDH1 antibody. Shown is the staining intensity (Y-axis) plotted against tumor type (X-axis). One experiment with at least three replicates per sample type. One-way ANOVA. *p<0.05, **p<0.01, ***p<0.001. Calculated significance is compared to normal liver. FIG. 19E: human HCC dataset was analyzed for the expression of highlighted genes using a web interface. Raldh1 is the primary isozyme expressed and is restricted to non-leukocytes within the tumors. Ptprc: protein-tyrosine phosphatase (CD45), a pan-leukocyte marker. Raldh: Retinaldehyde dehydrogenase. FIG. 19F: Transcript levels of the three Raldh isozymes in mouse HCC cell line (Hepa 1-6) and normal mouse liver. HCC cells predominantly express Raldh1 unlike normal liver where all three isozymes are detected.

FIGS. 20A-20F: Raldh1-INH show species-specific inhibition of RA production in HCC cells. FIG. 20A: SNU398 cells (human HCC cell line) were treated with the Raldh1-INH C86 or C91 for 24 hours. Representative two-color histograms shows loss of AldeRed fluorescence with Raldh1-INH whereas the bar graph shows quantitative changes in AldeRed-positive cells as a fraction of all cells when treated with different concentrations of the indicated Raldh1-INH. FIG. 20B: Median fluorescence intensity in AldeRed assay for different human HCC lines when treated with 100 nmol/L C86 for 24 hours. FIG. 20C: Representative two-color histograms show no change in AldeRed fluorescence when the murine HCC cell line (Hepa1-6) is treated with up to 1 mmol/L C86 or C91 for 24 hours. FIG. 20D: LC-MS for ATRA on Hepa1-6 (mouse HCC cell line) or SNU398 (human HCC cell line). Cells were treated with C86 (100 nmol/L for human or 1,000 nmol/L for mouse cells) or the nonspecific RALDH inhibitor WIN18446 (1,000 nmol/L) for 24 hours. WIN18446 inhibits both murine and human RALDH1 whereas C86 inhibition is specific to the human isozyme. Two independent experiments were performed with at least three replicates per experimental group. Unpaired t test, two-tailed. *, P<0.05; **, P<0.01; ***, P<0.001. FIG. 20E: SNU398 cells were treated with different concentrations of C86 or C91 for 3 days. Cell proliferation was measured by counting live cell numbers at each time point. FIG. 20F: Huh7 cells were treated with different concentrations of C86 for 3 days. Cell proliferation was measured by counting live cell numbers at each time point.

FIG. 21A: Transcript levels (RT-qPCR) of Raldh1, Raldh2 and Raldh3 in SNU398 cells treated with different concentrations of C86 or C91 for 24 hours. FIG. 21B: Homology modeling identifies differences in amino acid residues between human and murine RALDH1 at sites predicted to bind RALDH1-INH. FIG. 21C: SNU398 cells were treated with various concentrations of C86 or C91 for 24 hours. Cell viability was measured by flow cytometry (FCM) with 7-AAD staining (Y-axis indicates percentage of 7-AAD negative cells).

FIGS. 22A-22E: RALDH1 inhibition blocks RA-mediated effects of HCC on monocyte differentiation. FIGS. 22A-22B: Circulating primary human monocytes from donors (FIG. 22A) or murine monocytes from bone marrow (FIG. 22B) were cocultured with SNU398 (Human HCC) cells or treated with SNU398-conditioned media (CM) in a DC differentiation system (GM-CSF and IL-4). Two-color histograms show the frequencies of DCs (CD11c⁺CD1a⁺ for human and CD11c⁺MHCII⁺ for mouse). The presence of SNU398 or its CM suppressed DC differentiation, which is reversed when SNU398 cells are treated with C86. Three or more independent experiments were performed with at least three replicates per experimental group. Unpaired t test, two-tailed. FIG. 22C: Huh7 (human HCC) cells were subcutaneously injected into flanks of NU/J mice. 12 days later the tumor size was about 50 mm³ and C86 or vehicle treatment (10 mg/kg, i.p, daily injection) was started. On day 14, one million primary human monocytes (obtained from donors) were injected into these tumors. Five days after monocyte injection, mice were sacrificed and tumor tissue harvested for FCS analyses. Histogram shows DC (HLA-DR⁺CD1a⁺) differentiation of the injected human monocytes identified by human-specific CD45 (bar graph quantification on right). Three or more independent experiments were performed with at least three replicates per experimental group. Unpaired t test, two-tailed. FIG. 22D: Human monocytes were transplanted into Huh7 tumors similar to the strategy described for FIG. 22C and tumors were harvested 6 days after monocyte transplantation. The frequencies of the host (murine) DCs (CD45⁺F4/80⁻CD11C⁺MHCII-high) and macrophages (CD45⁺F4/80⁺) are quantified in a bar graph. Three or more independent experiments were performed with at least three replicates per experimental group. Unpaired t test, two-tailed. FIG. 22E: Circulating human primary monocytes from donors were cocultured with CM from Huh7-cells or the Huh7 cell line with Raldh1 deleted (RALDH1-KO) in a DC differentiation system (with GM-CSF and IL4). Shown are the representative FCM plot (histogram) and DC quantification (bar graph). Deletion of Raldh1 in tumor cells enhances DC differentiation from monocytes. Two independent experiments were performed with at least three replicates per experimental group. Unpaired t test, two-tailed. *, P<0.05; **, P<0.01; ***, P<0.001; n.s., not significant.

FIGS. 23A-23B: Total RNA was extracted from experiments described in FIGS. 22A-22B and the transcript levels of DC (Zbtb46 and Irf4) and macrophage (Mafb) associated genes were measured by RT-qPCR. FIG. 23A: results from human cells corresponding to experiment in FIG. 22A. FIG. 23B: results from mouse cells corresponding to experiment in FIG. 22B. Zbtb46 expression marks all DCs while Irf4 expression is induced upon monocyte to DC differentiation. Mafb expression marks macrophage differentiation and its levels are low in DCs. Three or more independent experiments with at least three replicates per sample type. Two-tailed T test. *p<0.05, **p<0.01, ***p<0.001.

FIGS. 24A-24E: Raldh1-INH blocks tumor supportive functions of monocytes and macrophages. FIG. 24A: Circulating primary human monocytes were collected from donors and cultured with MCSF (50 ng/mL) to generate macrophages. On day 3 of these cultures, DMSO or RA (100 nmol/L) was added to generate control monocyte-derived macrophages (Control-MoDM) or RA monocyte-derived macrophages (RA-MoDM), respectively. On day 7 of these cultures, macrophages were collected for each well after washing with 1×PBS, mixed with Huh7 cells at approximately 50:50 ratio, and the mix injected into the flanks of NU/J mice. Huh7 tumors cells that were not mixed with any macrophages before flank injection served as an additional control. Tumor volume was measured every 2 days (left graph). 19 days after tumor injection, mice were sacrificed and tumor weight (right graph) was measured. Tumor cells cotransplanted with RA-treated macrophages grew significantly faster than tumors cotransplanted with control macrophages or tumors transplanted without any macrophages. Three or more independent experiments were performed with at least three replicates per experimental group. Unpaired t test, two-tailed. FIG. 24B: Huh7 cells were subcutaneously injected to NU/J mice. When tumor size reached about 50 mm³, mice were intraperitoneally injected with liposomal clodronate (CloLipo; Liposoma, #C-015) and control liposomes (CtrlLipo; Liposoma, #P-015) at 200 mL/mice every 4 days. Tumor growth was monitored daily (left graph). 13 days after tumor cell transplantation, mice were sacrificed and tumor weight measured (right graph). Macrophage depletion slowed tumor growth. Two independent experiments were performed with at least three replicates per experimental group. Unpaired t test, two-tailed. FIG. 24C: Macrophages were generated from primary human monocytes by culturing them for 7 days with M-CSF. Macrophages were then collected, washed, and seeded into new well with indicated compounds with or without tumor-conditioned media (CM; TCM). Three days later, cells were harvested, counted, and stained with PI for FCS analyses. Shown are the numbers of live macrophages (y-axis) under different experimental conditions (x-axis). RA and tumor-CM significantly increased macrophage numbers over other conditions, an effect that is reversed with reduced RA (C86 treated TCM) or RA signaling blockade (BMS493). Three or more independent experiments were performed with at least three replicates per experimental group. Unpaired t test, two-tailed. FIG. 24D: Different human HCC lines (x-axis, first legend of each graph) were cocultured for 3 days with macrophages pretreated with various compounds (x-axis). Cell proliferation was measured by counting live cell numbers (y-axis). RA treated macrophages increased tumor cell numbers compared with control macrophages, an effect that is reversed with RA signaling blockade (BMS493). Three or more independent experiments were performed with at least three replicates per experimental group. Unpaired t test, two-tailed. FIG. 24E: Primary human monocytes were differentiated into macrophages with M-CSF alone (control) or M-CSF with BMS493, RA, or RA bBMS493 (x-axis). After 7 days, macrophages differentiated under these conditions were harvested and cocultured with Huh7 human HCC cells (1:10 tumor cell: macrophage) that were labeled with carboxyfluorescein diacetate succinimidyl ester (CFSE). 72 hours later, the cells were washed, counted, and analyzed by FCS. HCC cell division is indicated by the extent of CFSE dilution (y-axis). Three or more independent experiments were performed with at least three replicates per experimental group. Unpaired t test, two-tailed. *, P<0.05; **, P<0.01; *** P<0.001; n.s., not significant.

FIG. 25A: Tumors at the endpoint for experiment outlined in FIG. 24A. FIG. 25B: FCM plots of Macrophages (F4/80+ cells) in spleen tissues from experiment in FIG. 24B, showing macrophage depletion with CloLipo. FIG. 25C: Different human HCC lines were cultured for three days with conditioned medium (CM) from wells containing macrophages treated with different compounds (X-axis, macrophage CM). HCC cells exposed to the same compounds without conditioned media (X-axis, control CM) served as additional control. Viable HCC cell numbers were counted after three days (Y-axis). Three or more independent experiments with at least three replicates per sample type. Two-tailed T test. *p<0.05, **p<0.01, ***p<0.001.

FIGS. 26A-26H: Raldh1-INH suppresses HCC growth by altering macrophage functions. FIG. 26A: Huh7 tumor-bearing NU/J mice were treated with C86 (i.p, 10 mg/kg) every day starting when tumor size was approximately 50 mm³. Tumor volume was measured every 2 days. Bar graph on right shows tumor weight at endpoint. Three or more independent experiments were performed with at least three replicates per experimental group. Unpaired t test, two-tailed. FIG. 26B: AldeRed assay at endpoint on Huh7 tumor treated as described in FIG. 26A. Graph shows percentage of AldeRed-positive cells within CD45⁺ leukocytes and CD45⁻ cells (tumor⁺ stromal cells). Data show selective inhibition of RA in mostly the tumor cells. Three or more independent experiments were performed with at least three replicates per experimental group. Unpaired t test, two-tailed. FIG. 26C: Huh7 tumor-bearing nude mice were treated with different dose of C86 every day. Tumor volume was measured every 2-3 days. Bar graph on right shows tumor volume at experimental endpoint. Two independent experiments were performed with at least three replicates per experimental group. Unpaired t test, two-tailed. FIG. 26D: Body weight measured every 3-5 days for the experiment outlined in FIG. 26C. FIG. 26E: Parental Huh7 or RALDH1-KO Huh7 cells were implanted subcutaneously into NU/J mice and treated with C86 (i.p, 10 mg/kg) or vehicle (control) every day. RALDH1-KO tumors grew significantly slower than parental Huh7 and did not respond to C86 treatment. Bar graph on the right shows tumor weight at endpoint. Three or more independent experiments were performed with at least three replicates per experimental group. Unpaired t test, two-tailed. FIG. 26F: Huh7 tumor-bearing NU/J mice were treated with clodronate liposomes (CloLipo) and/or C86. CloLipo-treated tumors grew slower and did not respond to C86 treatment. Bar graph on the right shows tumor weight at endpoint. Two independent experiments were performed with at least three replicates per experimental group. Unpaired t test, two-tailed. FIG. 26G: Murine Hepa 1-6 tumors were implanted subcutaneously into C57BL6/J WT mice or LyMCre:RosadnRAR mice. Expression of the dominant negative RAR in myeloid cells slows tumor growth. Two independent experiments were performed with at least three replicates per experimental group. Unpaired t test, two-tailed. FIG. 26H: Huh7 cells were implanted subcutaneously into NU/J mice. When tumor size reached about 50 mm³, mice were intraperitoneally injected vehicle (control) or C86 daily with or without BMS493 treatment intratumorally every 3 days. BMS493 and C86 suppress tumor growth with the combination showing higher suppression than monotherapy. *, P<0.05; **, P<0.01; ***, P<0.001; n.s., not significant.

FIG. 27A: Huh1 tumor-bearing nude mice were treated with C86 (i.p, 10 mg/kg) every day. Three or more independent experiments with at least three replicates per sample type. Two-tailed T test. *p<0.05, **p<0.01, ***p<0.001. FIG. 27B: Equal numbers of Raldh1-KO Huh7 and parental Huh7 cells were seeded and viable cells counted over time. RALDH1 deficiency does not suppress cell proliferation in vitro. FIG. 27C: NU/J mice harboring flank Huh7 tumors were treated i.p with C86 daily or with liposomal chlodronate (CloLipo, 200 μl/mouse) every four days. Tumors were harvested 13 days post tumor induction and analyzed for the frequency (left graph) and number (right graph) of the macrophages (CD45+ F4/80+) under different treatment conditions (X-axis). Three or more independent experiments with at least three replicates per sample type. Two-tailed T test. *p<0.05, **p<0.01, ***p<0.001. FIG. 27D: Pictures depicting size differences amongst tumors in experiment outlined in FIG. 26G. FIG. 27E: FCS-based analyses of macrophage percentage (F4/80+ cells) within leukocytes (CD45+ cells) in tumors described in FIG. 26G. Two independent experiments were performed with at least three replicates per experimental group. Two-tailed T test. *p<0.05, **p<0.01, ***p<0.001.

FIGS. 28A-28G: C99 inhibits murine RALDH1 and suppresses murine HCC growth. FIG. 28A: AldeRed assay was performed on Hepa1-6 (murine HCC, left graph) and SNU398 (human HCC, right graph) cells with or without different concentration of C99 (x-axis). Shown is the percentage of AldeRed-positive cells (y-axis) after 24 hours exposure to C99. Murine RALDH1 is sensitive to C99. FIG. 28B: Hepa1-6 cells were treated with different concentrations of C99 in vitro and the number of viable cells were counted at different time points. C99 does not reduce cell viability in vitro. FIG. 28C: C57BL6/J mice were implanted subcutaneously with Hepa1-6 cells and the tumor-bearing mice. To prevent spontaneous rejection of this cell line, mice were treated with anti-CD3. Once the tumors reached around 50 mm³ size, mice were treated with C86 or C99 (i.p., 20 mg/kg) every day. Graph on the right shows tumor mass at end point. C99, but not C86, suppresses murine HCC growth. Two independent experiments were performed with at least three replicates per experimental group. Unpaired t test, two-tailed. FIG. 28D: Tumors in (FIG. 28C) were harvested at endpoint, single-cell suspension generated, and AldeRed assay was performed along with surface staining with immune cell markers. Shown is the percentage of AldeRed-positive cells in nonleukocytes (CD45 negative, mostly tumor cells) under the different treatment conditions. Two independent experiments were performed with at least three replicates per experimental group. Unpaired t test, two-tailed. FIG. 28E: FCS base frequency of Macrophages (F4/80⁺ cells) within CD45⁺ leukocytes in tumor tissues from experiment outlined in (FIGS. 28C-28D). Two independent experiments were performed with at least three replicates per experimental group. Unpaired t test, two-tailed. FIG. 28F: Hepa1-6 tumor-bearing NU/J mice were treated with chlodronate liposomes (CloLipo) and/or C86/C99. CloLipo treatment suppresses tumor growth and renders tumors insensitive to C99. Two independent experiments were performed with at least three replicates per experimental group. Unpaired t test, two-tailed. FIG. 28G: The murine fibrosarcoma (FS) cell line was implanted subcutaneously into C57BL6/J mice. Mice were treated with 25 mg/kg of C-99 or vehicle everyday starting one day after tumor cell transplantation. *, P<0.05; **, P<0.01; ***, P<0.001; ns, not significant.

FIG. 29A: IC₅₀ of C-86 and C-99 on the different human RALDH isozymes. FIG. 29B: Cell viability was measured by flow cytometry (FCM) with 7-AAD in experiment outlined in FIG. 28B. FIG. 29C: Tumors at endpoint from experiment in FIG. 28C. FIG. 29D: Tumor weights at the endpoint for FIG. 28G. One experiment with at least three replicates per experimental group. Two-tailed T test. *p<0.05, **p<0.01, ***p<0.001. FIGS. 29E-29F: ALDH1 was deleted with CRISPR/Cas9 in murine Hep55 HCC cell line (FIG. 29E). Two independent clones, 2 and 19, were selected (FIG. 29E). Both clones did not show any difference in growth in vitro when compared to parental WT cells (FIG. 29E). FIG. 29F: in contrast to in vitro growth, both clones showed dramatic growth suppression in vivo in a syngeneic (C57BL/6) flank transplantation model. Picture shows tumor size at endpoint corresponding to experiment show in FIG. 30A. FIG. 29G: Graph shows growth of tumors of the indicated genotypes and treatment syngeneically transplanted into C57BL/6 mice. Of note, there is a small but significant synergy between loss of RALDH1 and anti-PD1 treatment. Clone 2 was used for RALDH1-KO Hep55 cell line. Three or more independent experiments were performed with at least three replicates per experimental group. One-way ANOVA. *p<0.05, **p<0.01, ***p<0.001. FIG. 29H: Hep55 murine HCC cells were transplanted into the flanks of syngeneic C57BL/6 mice. Tumors were then harvested and single cell suspension were subjected to multi-parametric flow cytometry. Shown are the sequential (arrow) gating scheme leading to two macrophage subsets based on CD163—a marker for immunosuppressive macrophages. The histogram overlays on right (Mac1 subset; Mac2 subset) show the expression of anti-inflammatory (CD206 and Folate receptor B) and pro-inflammatory (MHCII and CD11C) markers in Mac1 and Mac2 subsets, supporting their pro- and anti-inflammatory function respectively. FIG. 29I: Flow cytometry-based comparison of macrophages in WT and RALDH1-knockout Hep55 tumors show increased MHCII+ pro-inflammatory macrophages in KO. Three or more independent experiments were performed with at least three replicates per experimental group. Two-tailed T test. *p<0.05, **p<0.01, ***p<0.001.

FIGS. 30A-30E: Pharmacokinetics of Raldh1-INH and synergy with immune checkpoint blockade. FIG. 30A: RALDH1 was deleted with CRISPR/Cas9 in the murine Hep55 HCC cell line. Two independent clones, 2 and 19, were selected on the basis of the confirmation of gene deletion. Cell lines of indicated genotypes were implanted subcutaneously into immunocompetent syngeneic C57BL6/J mice and tumor size monitored over time. Tumor size at experimental endpoint is shown in FIG. 29F. Three or more independent experiments were performed with at least three replicates per experimental group. One-way ANOVA. FIG. 30B: Tumors from (FIG. 30A) were harvested at endpoint (FIG. 29F) and T-cell infiltration analyzed by flow cytometry. Three or more independent experiments were performed with at least three replicates per experimental group. One-way ANOVA. FIG. 30C: Strategy for generating RALDH1-KO mice. Cas9 mRNA and the two guide RNAs (arrows) were microinjected into single-cell zygotes. Founders were identified by a PCR screening protocol designed to detect the approximately 36 kb deletion anticipated from dual cuts. The founders were then bred to C57BL/6 WTmice to “fix” the knockout allele. FIG. 30D: Confirmation of RALDH1 deletion in knockout mice through quantitative PCR performed with a murine RALDH1-specific TaqMan probe. Two independent experiments were performed with at least three replicates per experimental group. Unpaired t test, two-tailed. FIG. 30E: Serum from RALDH1-KO (⁻/⁻), heterozygous (⁺/⁻), and WT mice were used to perform serum toxicology analyses (standard tox panel no. 62794) through IDEXX bioanalytic services. Shown are a select few analytes from a larger panel. One experiment was performed with more than 5 replicates per genotype. One-way ANOVA. *, P<0.05; **, P<0.01; ***, P<0.001.

FIG. 31A: Completed blood count (CBC) was performed at IDEXX on whole blood collected from indicated RALDH1 genotypes (X-axis). One experiment with four replicates per experimental group. One-way ANOVA. *p<0.05, **p<0.01, ***p<0.001. FIG. 31B: Weights of mice of the indicated RALDH1 genotypes. Siblings were used for all genotypes to control for confounding factors. One experiment with four replicates per experimental group. One-way ANOVA. *p<0.05, **p<0.01, ***p<0.001. FIG. 31C: Pharmacokinetics of C86 delivered through an oral formulation with diet (chow). Diet (or PO) #A, B, and C (shown in the inbox of individual graphs) corresponds to drug dose of 10, 30, and 60 mg/kg respectively. Mice had free access to food and water during the 15-day study. Graph shows drug concentrations (Y-axis) is plotted against time (X-axis) in various tissue (header). FIG. 31D: Shows mouse weight (Y-axis) measured every day (X-axis) for the 15-day study described in FIG. 31A.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

Hepatocellular carcinoma (HCC) is a devastating disease with a projected annual incidence of around one million cases worldwide by 2025. Around 27,000 people die from this disease each year in the United States alone and advanced HCC has an abysmal 5-year survival rate of about 2%. Surgical resection and liver transplantation are preferred treatments for early-stage HCC, while locoregional interventions such as radiofrequency ablation and transarterial chemoembolization are used in unresectable cases. Advanced metastatic cases present substantial management challenges with a median survival of a few months. Recent progress in systemic therapies, which currently include immune checkpoint blockers, tyrosine kinase inhibitors (TKI), and angiogenesis inhibitors, have improved patient outcomes. Nonetheless, there is substantial room for improvement, especially with immunotherapy. Single-agent immune checkpoint blockade (ICB) elicits clinical responses in a minority of patients, suggesting the existence of other biological modulators of ICB responses.

Macrophages and dendritic cells (DCs) are the key antigen-presenting cells in solid tumors and, given the current limitations of immunotherapy, there has been increasing interest in therapeutically targeting them. These efforts are generally aimed at reducing the frequency of immunosuppressive macrophages, increasing the frequency of tumoricidal and pro-inflammatory macrophages, and enhancing the immunostimulatory activities of DCs. A number of approaches have been described that can achieve these effects in experimental models by targeting specific receptors and/or pathways in tumor-associated macrophage (TAMs) and DCs. In contrast, very little is known about whether and how monocyte differentiation into DCs vs. TAMs can be targeted for cancer immunotherapy. DCs and TAMs can also originate from non-monocyte precursors, embryonic progenitors arising from yolk sac and HSC-derived myeloid progenitors, respectively, but these progenitors are exceeding rare compared to abundant circulating monocytes. Furthermore, DCs and TAMs within the tumor microenvironment (TME) have a finite life-span requiring a continuous influx of progenitors. Thus, targeting monocyte differentiation represents a viable but largely unexplored therapeutic strategy in cancer immunotherapy.

As described elsewhere herein, some tumors produce retinoic acid (RA) that promotes differentiation of monocytes into immunosuppressive and tumor-promoting macrophages. Therefore, reducing RA production by tumor cells or inhibiting RA signaling in monocytes is a potential treatment approach in these tumors. Key barriers to implementation of this approach include identifying the tumors where this pathway is active and developing safe and effective inhibitors of the RA pathway. As described herein, HCCs produce high levels of RA through overexpression of RALDH1, which is one of the three enzymes that catalyzes RA production. RA production in HCC was abrogated with certain exemplary RALDH1 inhibitors (Raldh1-INH). These inhibitors suppressed tumor growth in multiple mouse models of human and murine HCC. Using genetic and pharmacologic tools, as described herein, the HCC-suppressive effects of Raldh1-INH are driven by altered macrophage numbers and function as well as increased infiltration of tumors by activated T cells. Pharmacological and toxicological analyses revealed a favorable profile of Raldh1-INH for potential clinical use, which was also supported by observations in newly generated RALDH1 knockout (RALDH1-KO) mice. These findings provide proof of concept for the use of Raldh1-INH in HCC and establish the scientific premise for the development of isozyme-specific RALDH inhibitors as a new strategy in cancer immunotherapy.

### Definitions

The term “about” as used herein can allow for a degree of variability in a value or range, for example, within 10%, within 5%, or within 1% of a stated value or of a stated limit of a range, and includes the exact stated value or range.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the compounds and compositions described herein are administered orally.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆) alkyl, such as, but not limited to, ethyl, methyl, isopropyl, isobutyl, n-pentyl, n-hexyl and cyclopropylmethyl. Linear, branched, and/or cyclic moieties having a number of carbon atoms designated in a range (e.g., C₁-C₆) should be interpreted in a flexible manner to include not only the numerical values explicitly recited as the limits of the range, but also to include all the individual numerical values or sub-ranges encompassed within that range as if each numerical value and sub-range is explicitly recited (e.g., C₁-C₆ includes individual values C₁, C₂, C₃, C₄, C₅, and C₆, and non-limiting exemplary ranges C₁-C₅ and C₂-C₆, inter alia).

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As described herein, the term “chimeric antigen receptor (CAR) T-cell” refers to T-cells that have been genetically engineered to provide an artificial T-cell receptor for use in immunotherapy.

The term “co-administered” as used herein is meant simultaneous administration in the same formulation or in two different formulations via the same or different routes or sequential administration by the same or different routes. By “sequential” administration is meant a time difference of from seconds, minutes, hours or days between the administration of the two or more separate compounds.

The term “combination therapy” means the administration of two or more therapeutic agents to treat a therapeutic condition or disorder described in the present disclosure. Such administration encompasses co-administration of these therapeutic agents in a substantially simultaneous manner, such as in a single formulation (e.g., a capsule or injection) having a fixed ratio of active ingredients or in multiple, separate dosage forms for each active ingredient. In addition, such administration also encompasses use of each type of therapeutic agent in a sequential manner. In either case, the treatment regimen will provide beneficial effects of the drug combination in treating the conditions or disorders described herein.

The term “comprising” is used in this specification to mean including the feature(s) or act(s) followed thereafter, without excluding the presence of one or more additional features or acts.

The terms “effective amount” or “therapeutically effective amount” as used herein, refer to a sufficient amount of at least one compound being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is a decrease in the growth of, the killing of, or the inducing of apoptosis in at least one abnormally proliferating cell, e.g., a cancer cell. In certain instances, an “effective amount” for therapeutic uses is the amount of the composition comprising a compound as set forth herein required to provide a clinically significant decrease in a disease. An appropriate “effective” amount in any individual case is determined using any suitable technique, such as a dose escalation study.

As used herein, the term “halo” or “halogen” alone or as part of another substituent refers to, unless otherwise stated, a fluorine, chlorine, bromine, or iodine atom.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system having the number of carbon atoms designated (i.e., C₂-C₈ refers to a cyclic group comprising a ring group consisting of 2 to 8 carbon atoms) and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

The term “immunostimulator” as used herein refers to a substance, non-limiting examples including small molecules, macromolecules (e.g. proteins), and biologics (e.g. T-cells), that are capable of modulating an immune response (e.g. stimulate and/or engage in an immune response).

As used herein, the term “increase” or the related terms “increased,” “enhance” or “enhanced” may refer to a statistically significant increase, and the terms “decreased,” “suppressed,” or “inhibited” to a statistically significant decrease. For the avoidance of doubt, an increase generally refers to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control, baseline, or prior-in-time value. Inhibition generally refers to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease over the control value.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

The term “pharmaceutically acceptable salt” may refer to salts prepared from pharmaceutically acceptable non-toxic acids or bases including inorganic acids and bases and organic acids and bases. When the compounds disclosed in this specification are basic, salts may be prepared from pharmaceutically acceptable non-toxic acids including inorganic and organic acids. Suitable pharmaceutically acceptable acid addition salts for the compounds disclosed in this specification include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

As used herein, a “pharmaceutically effective amount,” “therapeutically effective amount” or “effective amount” of a compound is that amount of compound that is sufficient to provide a beneficial effect to the subject to which the compound is administered.

The term “prevent,” “preventing” or “prevention” as used herein means avoiding or delaying the onset of symptoms associated with a disease or condition in a subject that has not developed such symptoms at the time the administering of an agent or compound commences. Disease, condition and disorder are used interchangeably herein.

The term “retinoic acid modulator” as used herein refers to a substance which acts to increase or decrease the abundance or the influence of retinoic acid present in a subject by any of a number of mechanisms. In certain embodiments, a retinoic acid modulator may decrease the abundance of retinoic acid. In certain embodiments, the decrease of retinoic acid abundance may occur by inhibition of retinoic acid biosynthesis.

By the term “specifically bind” or “specifically binds” as used herein is meant that a first molecule preferentially binds to a second molecule (e.g., a particular receptor or enzyme), but does not necessarily bind only to that second molecule.

As used herein, the term “subject” means all mammals including humans. Examples of subjects include humans, mice, primates, cows, dogs, cats, goats, sheep, pigs, and rabbits. In some embodiments, the subject is a human.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

As used herein, the term “substituted” refers to that an atom or group of atoms has replaced hydrogen as the substituent attached to another group.

As used herein, the term “substituted alkyl,” “substituted cycloalkyl,” “substituted alkenyl” or “substituted alkynyl” refers to alkyl, cycloalkyl, alkenyl or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, preventing, delaying onset of, delaying recurrence of, abating or ameliorating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

In certain embodiments, each occurrence of alkyl or cycloalkyl is independently optionally substituted with at least one substituent selected from the group consisting of C₁-C₆ alkyl, halo, —OR, phenyl (thus yielding, in non-limiting examples, optionally substituted phenyl-(C₁-C₃ alkyl), such as, but not limited to, benzyl or substituted benzyl) and —N(R)(R), wherein each occurrence of R is independently H, C₁-C₆ alkyl or C₃-C₈ cycloalkyl. In other embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R)(R), —NO₂, —S(═O)₂N(R)(R), acyl, and C₁-C₆ alkoxycarbonyl, wherein each occurrence of R is independently H, C₁-C₆ alkyl or C₃-C₈ cycloalkyl. In yet other embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R)(R), and C₁-C₆ alkoxycarbonyl, wherein each occurrence of R is independently H, C₁-C₆ alkyl or C₃-C₈ cycloalkyl.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual sub-combination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

### Methods

The present disclosure provides a method of treating, preventing, and/or ameliorating a solid tumor in a subject in need thereof, the method comprising administering to the subject a pharmaceutically effective amount of:
(a) at least one immunostimulator; and
(b) a retinaldehyde dehydrogenase 1 (RALDH1) inhibitor, wherein the RALDH1 inhibitor is selected from the group consisting of:
(i) a compound of formula (I), or a salt, solvate, prodrug, stereoisomer, tautomer, or isotopologue thereof:

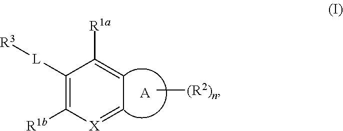

wherein:
R^{1a} is selected from the group consisting of optionally substituted C₂-C₈ heterocyclyl, optionally substituted phenyl, and optionally substituted C₅-C₈ cycloalkenyl,
   wherein each optional substituent in R^{1a} is independently selected from the group consisting of C₁-C₆ alkyl, C₃-C₆ cycloalkyl, C₁-C₆ alkoxy, optionally substituted phenyl, optionally substituted C₂-C₈ heterocyclyl, halogen, OH, N(R^{a})(R^{b}), NO₂, CN, C(═O)R^{a}, C(═O)OR^{a}, C(═O)N(R^{a})(R^{b}), S(═O)₂R^{a}, S(═O)₂OR^{a}, S(═O)₂N(R^{a})(R^{b}) OC(═O)R^{a}, N(R^{a})=S(═O)(R^{b})(R^{c}), S(═O)(=NR^{a})R^{b}, and N(R^{a})C(═O)R^{b},
      wherein each optional substituent is optionally substituted with at least one substituent selected from the group consisting of C₁-C₃ alkyl, C₁-C₃ alkoxy, halogen, CN, and NO₂, and
   wherein two vicinal or geminal optional substituents in R^{1a} may combine with the atoms to which they are bound to form a C₂-C₈ heterocyclyl or C₃-C₈ cycloalkyl;
R^{1b} and R^{1c}, if present, are each independently selected from the group consisting of H, C₁-C₆ alkyl, C₁-C₆ alkoxy, halogen, OH, N(R^{a})(R^{b}), NO₂, and CN;
each occurrence of R² is independently selected from the group consisting of C₁-C₆ alkyl, C₁-C₆ alkoxy, C₁-C₃ haloalkoxy, C₁-C₆ hydroxyalkyl, halogen, NO₂, and CN;
R³ is selected from the group consisting of optionally substituted C₂-C₈ heterocyclyl, optionally substituted phenyl, N(R^{a})(optionally substituted C₃-C₈ cycloalkyl), and N(R^{a})(optionally substituted C₂-C₈ heterocyclyl),
   wherein each optional substituent in R³ is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)OR^{a}, C(═O)N(R^{a})(R^{b}), S(═O)₂R^{a}, S(═O)₂OR^{a}, S(═O)₂N(R^{a})(R^{b}), OC(═O)R^{a}, and N(R^{a})C(═O)R^{b};
A is selected from the group consisting of optionally substituted C₆-C₁₀ aryl and optionally substituted C₂-C₈ heterocyclyl,
   wherein each optional substituent in A is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)OR^{a}, C(═O)N(R^{a})(R^{b}), S(═O)₂R^{a}, S(═O)₂OR^{a}, S(═O)₂N(R^{a})(R^{b}), OC(═O)R^{a}, and N(R^{a})C(═O)R^{b};
L is selected from the group consisting of —CH₂—, —C(═O)—, and —S(═O)₂—;
X is selected from the group consisting of N and CR^{1c};
n is an integer selected from the group consisting of 0, 1, 2, 3, and 4; and
each occurrence of R^{a}, R^{b}, and R^{c} is independently selected from the group consisting of H, C₁-C₆ alkyl, C₂-C₆ alkenyl, C₂-C₆ alkynyl, C₃-C₈ cycloalkyl, and C₂-C₈ heterocyclyl; and
(ii) a compound selected from the group consisting of:
   1-benzylindoline-2,3-dione;
   ethyl 2-((4-oxo-3-(3-(pyrrolidin-1-yl)propyl)-3,4-dihydrobenzo[4,5]thieno[3,2-d]pyrimidin-2-yl)thio)acetate;
   2-((2-(sec-butyl)-3-oxo-2,3-dihydroimidazo[1,2-c]quinazolin-5-yl)thio)-N-(o-tolyl)butanamide; and
   8-((4-(cyclopropanecarbonyl)piperazin-1-yl)methyl)-7-isopentyl-1,3-dimethyl-3,7-dihydro-1H-purine-2,6-dione,
   or a salt, solvate, prodrug, stereoisomer, tautomer, or isotopologue thereof.

In certain embodiments, the C₂-C₈ heterocyclyl in R^{1a} is thiophen-2-yl. In certain embodiments, the C₂-C₈ heterocyclyl in R^{1a} is thiophen-3-yl.

In certain embodiments, the optionally substituted C₆-C₁₀ aryl in A is optionally substituted phenyl. In certain embodiments, the optionally substituted C₂-C₈ heterocyclyl is optionally substituted C₂-C₅ heterocyclyl.

In certain embodiments, the RALDH1 inhibitor is a compound of formula (I).

In certain embodiments, the compound of formula (I) is selected from the group consisting of:

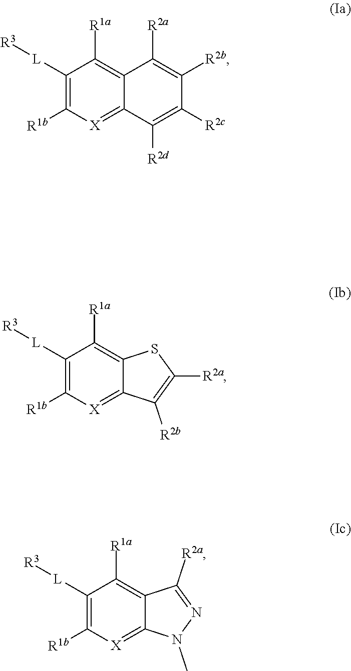

wherein:
R^{2a}, R^{2b}, R^{2c}, and R^{2d}, if present, are each independently selected from the group consisting of H, C₁-C₆ alkyl, C₁-C₆ alkoxy, and halogen.

In certain embodiments, R^{2a}, R^{2b}, R^{2c}, and R^{2d}, if present, are each independently selected from the group consisting of H, Me, OMe, F, and Cl.

In certain embodiments, X is N.

In certain embodiments, R^{1a} is selected from the group consisting of:

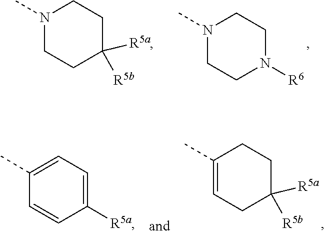

wherein:
R^{5a} and R^{5b}, if present, are each independently selected from the group consisting of C₁-C₆ alkyl, C₁-C₆ alkoxy, C₃-C₆ cycloalkyl, phenyl, thiophen-2-yl, thiophen-3-yl, and CN,
   wherein each substituent in R^{5a} and R^{5b} is optionally substituted with at CN, and
   wherein R^{5a} and R^{5b}, if present, may combine with the atoms to which they are bound to form a C₂-C₆ heterocyclyl or C₃-C₆ cycloalkyl; and
R⁶ is S(═O)₂R^{a}.

In certain embodiments, R^{5a} is Me. In certain embodiments, R^{5a} is t-tBu. In certain embodiments, R^{5a} is 1-cyanocyclopropyl. In certain embodiments, R^{5a} is 1-cyanocyclopentyl. In certain embodiments, R^{5a} is phenyl. In certain embodiments, R^{5a} is CN. In certain embodiments, R^{5a} is 1-cyanocyclobutyl. In certain embodiments, R^{5a} is 1-cyanocyclohexyl. In certain embodiments, R^{5a} is thiophen-2-yl. In certain embodiments, R^{5a} is thiophen-3-yl.

In certain embodiments, R^{5b} is Me. In certain embodiments, R^{5b} is t-tBu. In certain embodiments, R^{5b} is 1-cyanocyclopropyl. In certain embodiments, R^{5b} is 1-cyanocyclopentyl. In certain embodiments, R^{5b} is phenyl. In certain embodiments, R^{5b} is CN. In certain embodiments, R^{5b} is 1-cyanocyclobutyl. In certain embodiments, R^{5b} is 1-cyanocyclohexyl. In certain embodiments, R^{5b} is thiophen-2-yl. In certain embodiments, R^{5b} is thiophen-3-yl.

In certain embodiments, R⁶ is ethenylsulfonyl.

In certain embodiments, R^{1a} is

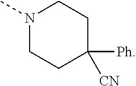

In certain embodiments, R^{1a} is

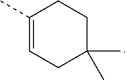

In certain embodiments, R^{1a} is

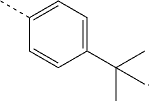

In certain embodiments, R^{1a} is

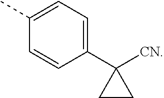

In certain embodiments, R^{1a} is

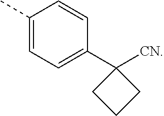

In certain embodiments, R^{1a} is

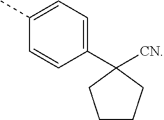

In certain embodiments, R^{1a} is

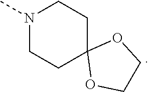

In certain embodiments, R^{1a} is

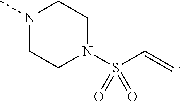

In certain embodiments, R^{1a} is

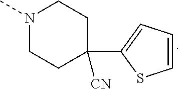

In certain embodiments, R^{1b} is H.

In certain embodiments, R³ is selected from the group consisting of:

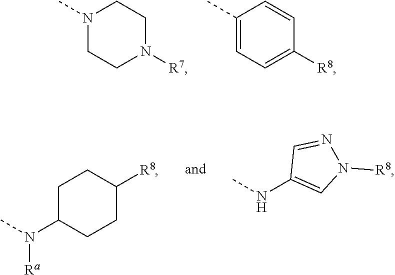

wherein:
R⁷ is selected from the group consisting of C(═O)R^{a} and S(═O)₂R^{a}; and
R⁸ is selected from the group consisting of C₁-C₆ alkoxy, C₁-C₆ hydroxyalkyl, and OH.

In certain embodiments, R⁷ is cyclopropylcarbonyl. In certain embodiments, R⁷ is methylsulfonyl. In certain embodiments, R⁷ is dimethylaminosulfonyl. In certain embodiments, R⁷ is dimethylaminocarbonyl.

In certain embodiments, R⁸ is methoxy. In certain embodiments, R⁸ is 2-hydroxyethyl.

In certain embodiments, R⁸ is OH.

In certain embodiments, R³ is

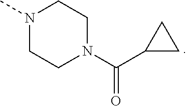

In certain embodiments, R³ is

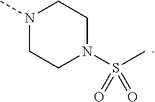

In certain embodiments, R³ is

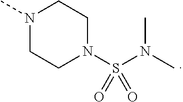

In certain embodiments, R³ is

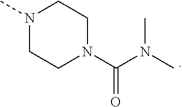

In certain embodiments, R³ is

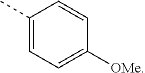

In certain embodiments, R³ is

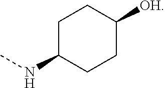

In certain embodiments, R³ is

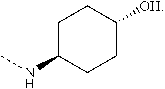

In certain embodiments, R³ is

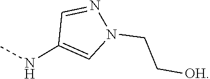

In certain embodiments, the compound of formula (I) is selected from the group consisting of:
8-(6-methoxy-3-((4-methoxyphenyl)sulfonyl)quinolin-4-yl)-1,4-dioxa-8-azaspiro[4.5]decane;
1-(3-(4-(cyclopropanecarbonyl)piperazine-1-carbonyl)-6-fluoroquinolin-4-yl)-4-phenylpiperidine-4-carbonitrile;
(4-(cyclopropanecarbonyl)piperazin-1-yl)(4-(4,4-dimethylcyclohex-1-en-1-yl)-6-fluoroquinolin-3-yl)methanone;
(4-(4-(tert-butyl)phenyl)-6-fluoroquinolin-3-yl)(4-(cyclopropanecarbonyl)piperazin-1-yl)methanone;
1-(4-(3-(4-(cyclopropanecarbonyl)piperazine-1-carbonyl)-6-fluoroquinolin-4-yl)phenyl)cyclopropanecarbonitrile;
1-(6-fluoro-3-(4-(methylsulfonyl)piperazine-1-carbonyl)quinolin-4-yl)-4-phenylpiperidine-4-carbonitrile;
(4-(4,4-dimethylcyclohex-1-en-1-yl)-6-fluoroquinolin-3-yl)(4-(methylsulfonyl)piperazin-1-yl)methanone;
(4-(4-(tert-butyl)phenyl)-6-fluoroquinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclopropane-1-carbonitrile;
4-(4-cyano-4-phenylpiperidin-1-yl)-6-fluoro-N-((1r,4r)-4-hydroxycyclohexyl)quinoline-3-carboxamide;
(6-fluoro-4-(4-(vinylsulfonyl)piperazin-1-yl)quinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclobutane-1-carbonitrile;
1-(4-(6-fluoro-3-(4-(methylsulfonyl)piperazine-1-carbonyl)quinolin-4-yl)phenyl)cyclopentane-1-carbonitrile;
4-(4-(4-cyano-4-phenylpiperidin-1-yl)-6-fluoroquinoline-3-carbonyl)-N,N-dimethylpiperazine-1-sulfonamide;
4-(4-(4-cyano-4-phenylpiperidin-1-yl)-6-fluoroquinoline-3-carbonyl)-N,N-dimethylpiperazine-1-carboxamide;
1-(6-chloro-3-(4-(methylsulfonyl)piperazine-1-carbonyl)quinolin-4-yl)-4-phenylpiperidine-4-carbonitrile;
1-(6-chloro-3-(4-(cyclopropanecarbonyl)piperazine-1-carbonyl)quinolin-4-yl)-4-phenylpiperidine-4-carbonitrile;
1-(6-(4-(methylsulfonyl)piperazine-1-carbonyl)thieno[3,2-b]pyridin-7-yl)-4-phenylpiperidine-4-carbonitrile;
1-(6-(4-(cyclopropanecarbonyl)piperazine-1-carbonyl)thieno[3,2-b]pyridin-7-yl)-4-phenylpiperidine-4-carbonitrile;
1-(6-methoxy-3-(4-(methylsulfonyl)piperazine-1-carbonyl)quinolin-4-yl)-4-phenylpiperidine-4-carbonitrile;
1-(7-methoxy-3-(4-(methylsulfonyl)piperazine-1-carbonyl)quinolin-4-yl)-4-phenylpiperidine-4-carbonitrile;
1-(6,7-difluoro-3-(4-(methylsulfonyl)piperazine-1-carbonyl)quinolin-4-yl)-4-phenylpiperidine-4-carbonitrile;
1-(6,8-difluoro-3-(4-(methylsulfonyl)piperazine-1-carbonyl)quinolin-4-yl)-4-phenylpiperidine-4-carbonitrile;
4-(4-(4-(1-cyanocyclopropyl)phenyl)-6-fluoroquinoline-3-carbonyl)-N,N-dimethylpiperazine-1-sulfonamide;
4-(4-(4-(1-cyanocyclopropyl)phenyl)-6-fluoroquinoline-3-carbonyl)-N,N-dimethylpiperazine-1-carboxamide;
4-(6-chloro-4-(4-(1-cyanocyclopropyl)phenyl)quinoline-3-carbonyl)-N,N-dimethylpiperazine-1-sulfonamide;
1-(4-(6-chloro-3-(4-(cyclopropanecarbonyl)piperazine-1-carbonyl)quinolin-4-yl)phenyl)cyclopropane-1-carbonitrile;
1-(4-(6-(4-(methylsulfonyl)piperazine-1-carbonyl)thieno[3,2-b]pyridin-7-yl)phenyl)cyclopropane-1-carbonitrile;
1-(4-(6-(4-(cyclopropanecarbonyl)piperazine-1-carbonyl)thieno[3,2-b]pyridin-7-yl)phenyl)cyclopropane-1-carbonitrile;
1-(4-(6-methoxy-3-(4-(methylsulfonyl)piperazine-1-carbonyl)quinolin-4-yl)phenyl)cyclopropane-1-carbonitrile;
1-(4-(7-methoxy-3-(4-(methylsulfonyl)piperazine-1-carbonyl)quinolin-4-yl)phenyl)cyclopropane-1-carbonitrile;
1-(4-(6,7-difluoro-3-(4-(methylsulfonyl)piperazine-1-carbonyl)quinolin-4-yl)phenyl)cyclopropane-1-carbonitrile;
1-(4-(6,8-difluoro-3-(4-(methylsulfonyl)piperazine-1-carbonyl)quinolin-4-yl)phenyl)cyclopropane-1-carbonitrile;
4-(4-(1-cyanocyclopropyl)phenyl)-6-fluoro-N-((1r,4r)-4-hydroxycyclohexyl)quinoline-3-carboxamide;
4-(4-(1-cyanocyclopropyl)phenyl)-6-fluoro-N-(1-(2-hydroxyethyl)-1H-pyrazol-4-yl)quinoline-3-carboxamide; and
4-(4-cyano-4-phenylpiperidin-1-yl)-6-fluoro-N-(1-(2-hydroxyethyl)-1H-pyrazol-4-yl)quinoline-3-carboxamide.

In certain embodiments, the solid tumor is a carcinoma.

In certain embodiments, the carcinoma comprises human hepatocellular carcinoma (HCC) cells.

In certain embodiments, RALDH1 is overexpressed in the solid tumor.

In certain embodiments, expression of Raldh1 and Raldh2 or Raldh1 and Raldh3 in the solid tumor has a ratio ranging from about 10000:1 to about 2:1. In certain embodiments, expression of Raldh1 and Raldh2 or Raldh1 and Raldh3 in the solid tumor has a ratio selected from the group consisting of about 10000:1, 9000:1, 8000:1, 7000:1, 6000:1, 5000:1, 4000:1, 3000:1, 2000:1, and 1000:1. In certain embodiments, expression of Raldh1 and Raldh2 or Raldh1 and Raldh3 in the solid tumor has a ratio selected from the group consisting of about 900:1, 800:1, 700:1, 600:1, 500:1, 400:1, 300:1, 200:1, and 100:1. In certain embodiments, expression of Raldh1 and Raldh2 or Raldh1 and Raldh3 in the solid tumor has a ratio selected from the group consisting of about 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 4:1, 3:1, and 2:1.

In certain embodiments, the immunostimulator is at least one selected from the group consisting of an immune checkpoint inhibitor, chimeric antigen receptor (CAR) T-cells, T-cells engineered to express specific TCR targeted tumor antigens (TCR-transgenic), ex vivo expanded T-cells, and bispecific T-cell engagers (BiTE).

In certain embodiments, the immunostimulator is an immune checkpoint inhibitor.

In certain embodiments, the immune checkpoint inhibitor is selected from the group consisting of an anti-PD1 antibody, an anti-PD-L1 antibody, an anti-CTLA4 antibody, any fragment thereof, and any combinations thereof. In certain embodiments, the immune checkpoint inhibitor is anti-PD1 antibody. In certain embodiments, the immune checkpoint inhibitor is an anti-PD-L1 antibody. In certain embodiments, the immune checkpoint inhibitor is an anti-CTLA4 antibody.

In certain embodiments, the immunostimulator is CAR T-cells. In certain embodiments, the CAR T-cells are administered intravenously. In certain embodiments, the CAR T-cells are administered as a CAR T-cell therapy.

In certain embodiments, the subject is administered an immune checkpoint inhibitor and chimeric antigen receptor (CAR) T-cells.

In certain embodiments, the method further comprises administering to the subject at least one selected from the group consisting of a retinoic acid receptor (RAR) inhibitor and a retinoid X receptor (RXR) inhibitor.

In certain embodiments, the RAR inhibitor is selected from the group consisting of AGN 193109, BMS 195614, BMS 493, CD 2665, ER 50891, LE 135, LY 2955303, MM 11253, any salt or solvate thereof, and any combinations thereof.

In certain embodiments, the RXR inhibitor is selected from the group consisting of HX 531, PA 452, and UVI 3003, any salt or solvate thereof, and any combinations thereof.

In certain embodiments, the RALDH1 inhibitor and the immunostimulator are administered to the subject simultaneously or sequentially.

In certain embodiments, the subject is a mammal. In certain embodiments, the mammal is a human.

### Retinoic Acid Modulators

The compounds of the disclosure may possess one or more stereocenters, and each stereocenter may exist independently in either the (R) or (S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

In certain embodiments, the compounds of the invention exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed invention. The compounds of the invention may contain any of the substituents, or combinations of substituents, provided herein.

Compounds of the present teachings can be prepared from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

### RALDH1 Inhibitors

The RALDH1 inhibitors of the present invention inhibit RALDH1 with high selectivity over other RALDHs (i.e. RALDH2 and RALDH3), and may be chemically synthesized by methods known in the art, or they may be purchased from commercial sources. The RALDH1 inhibitors are not limited to the compounds described herein, but comprise any RALDH1 inhibitor known in the art, including Yang et. al (J. Med. Chem. 2018, 61(11):4883-4903), which is incorporated herein by reference. Non-limiting examples of RALDH1 inhibitors contemplated for use in the present disclosure include any of the compounds provided in Table 1, or a salt, solvate, prodrug, stereoisomer, or isotopologue thereof.

TABLE 1

| Compound | Structure | Name |
|---|---|---|
| C-1 | 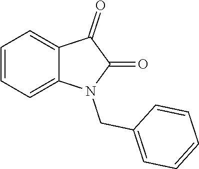 [SMILES: O=C1C(=O)N(Cc2ccccc2)c2ccccc21] | 1-benzylindoline-2,3-dione |
| C-2 | 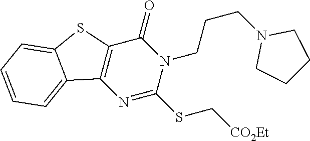 [SMILES: CCOC(=O)CSc1nc2c(sc3ccccc32)c(=O)n1CCCN1CCCC1] | ethyl 2-((4-oxo-3-(3-(pyrrolidin-1- yl)propyl)-3,4- dihydrobenzo[4,5]thieno[3,2- d]pyrimidin-2-yl)thio)acetate |
| C-3 | 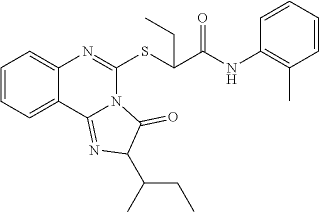 [SMILES: CCC(SC1=Nc2ccccc2C2=NC(C(C)CC)C(=O)N12)C(=O)Nc1ccccc1C] | 2-((2-(sec-butyl)-3-oxo-2,3- dihydroimidazo[1,2-c]quinazolin-5- yl)thio)-N-(o-tolyl)butanamide |
| C-4 | 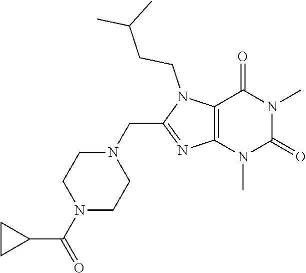 [SMILES: CC(C)CCn1c(CN2CCN(C(=O)C3CC3)CC2)nc2c1c(=O)n(C)c(=O)n2C] | 8-((4- (cyclopropanecarbonyl)piperazin-1- yl)methyl)-7-isopentyl-1,3- dimethyl-3,7-dihydro-1H-purine- 2,6-dione |
| C-5 | 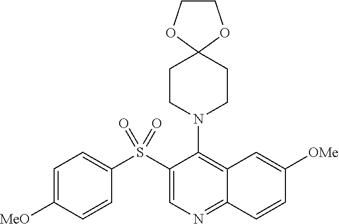 [SMILES: COc1ccc(S(=O)(=O)c2cnc3ccc(OC)cc3c2N2CCC3(CC2)OCCO3)cc1] | 8-(6-methoxy-3-((4- methoxyphenyl)sulfonyl)quinolin- 4-yl)-1,4-dioxa-8- azaspiro[4.5]decane |
| C-78 | 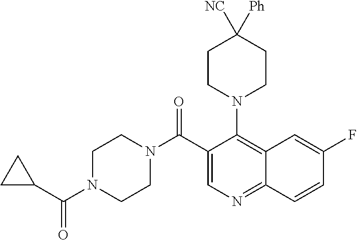 [SMILES: N#CC1(c2ccccc2)CCN(c2c(C(=O)N3CCN(C(=O)C4CC4)CC3)cnc3ccc(F)cc23)CC1] | 1-(3-(4- (cyclopropanecarbonyl)piperazine- 1-carbonyl)-6-fluoroquinolin-4-yl)- 4-phenylpiperidine-4-carbonitrile |
| C-80 | 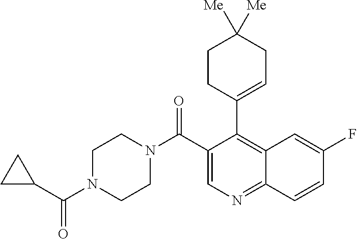 [SMILES: CC1(C)CC=C(c2c(C(=O)N3CCN(C(=O)C4CC4)CC3)cnc3ccc(F)cc23)CC1] | (4- (cyclopropanecarbonyl)piperazin-1- yl)(4-(4,4-dimethylcyclohex-1-en-1- yl)-6-fluoroquinolin-3- yl)methanone |
| C-81 | 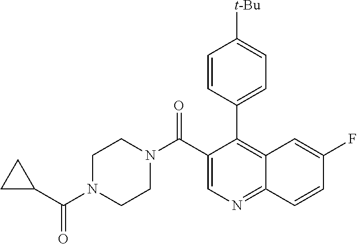 [SMILES: CC(C)(C)c1ccc(-c2c(C(=O)N3CCN(C(=O)C4CC4)CC3)cnc3ccc(F)cc23)cc1] | (4-(4-(tert-butyl)phenyl)-6- fluoroquinolin-3-yl)(4- (cyclopropanecarbonyl)piperazin-1- yl)methanone |
| C-83 | 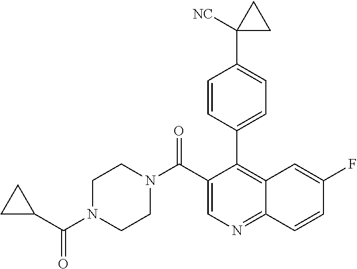 [SMILES: N#CC1(c2ccc(-c3c(C(=O)N4CCN(C(=O)C5CC5)CC4)cnc4ccc(F)cc34)cc2)CC1] | 1-(4-(3-(4- (cyclopropanecarbonyl)piperazine- 1-carbonyl)-6-fluoroquinolin-4- yl)phenyl)cyclopropane-1- carbonitrile |
| C-86 | 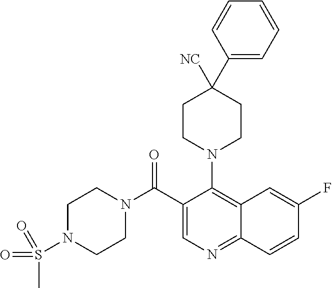 [SMILES: CS(=O)(=O)N1CCN(C(=O)c2cnc3ccc(F)cc3c2N2CCC(C#N)(c3ccccc3)CC2)CC1] | 1-(6-fluoro-3-(4- (methylsulfonyl)piperazine-1- carbonyl)quinolin-4-yl)-4- phenylpiperidine-4-carbonitrile |
| C-88 | 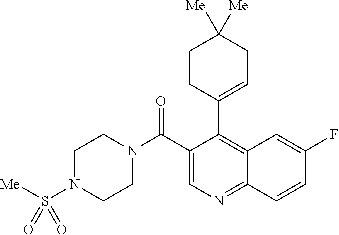 [SMILES: CC1(C)CC=C(c2c(C(=O)N3CCN(S(C)(=O)=O)CC3)cnc3ccc(F)cc23)CC1] | (4-(4,4-dimethylcyclohex-1-en-1- yl)-6-fluoroquinolin-3-yl)(4- (methylsulfonyl)piperazin-1- yl)methanone |
| C-89 | 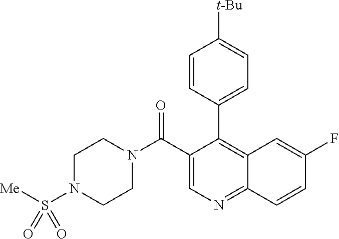 [SMILES: CC(C)(C)c1ccc(-c2c(C(=O)N3CCN(S(C)(=O)=O)CC3)cnc3ccc(F)cc23)cc1] | (4-(4-(tert-butyl)phenyl)-6- fluoroquinolin-3-yl)(4- (methylsulfonyl)piperazin-1- yl)methanone |
| C-91 | 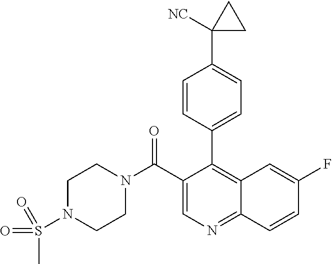 [SMILES: CS(=O)(=O)N1CCN(C(=O)c2cnc3ccc(F)cc3c2-c2ccc(C3(C#N)CC3)cc2)CC1] | 1-(4-(6-fluoro-3-(4- (methylsulfonyl)piperazine-1- carbonyl)quinolin-4- yl)phenyl)cyclopropane-1- carbonitrile |
| C-97 | 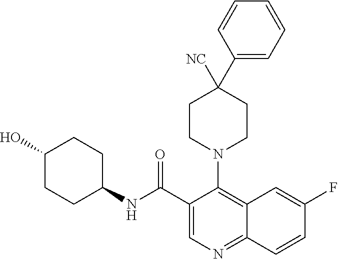 [SMILES: N#CC1(c2ccccc2)CCN(c2c(C(=O)N[C@H]3CC[C@H](O)CC3)cnc3ccc(F)cc23)CC1] | 4-(4-cyano-4-phenylpiperidin-1-yl)- 6-fluoro-N-((1r,4r)-4- hydroxycyclohexyl)quinoline-3- carboxamide |
| C-99 | 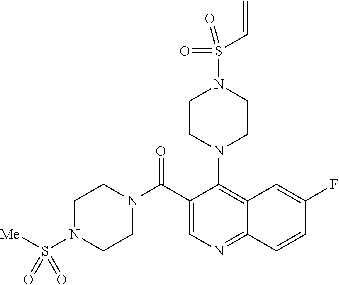 [SMILES: C=CS(=O)(=O)N1CCN(c2c(C(=O)N3CCN(S(C)(=O)=O)CC3)cnc3ccc(F)cc23)CC1] | (6-fluoro-4-(4- (vinylsulfonyl)piperazin-1- yl)quinolin-3-yl)(4- (methylsulfonyl)piperazin-1- yl)methanone |
| C-105 | 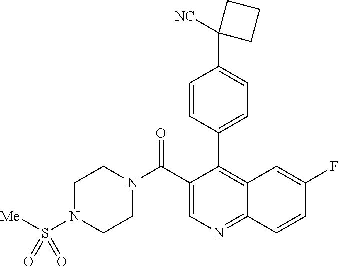 [SMILES: CS(=O)(=O)N1CCN(C(=O)c2cnc3ccc(F)cc3c2-c2ccc(C3(C#N)CCC3)cc2)CC1] | 1-(4-(6-fluoro-3-(4- (methylsulfonyl)piperazine-1- carbonyl)quinolin-4- yl)phenyl)cyclobutane-1- carbonitrile |
| C-106 | 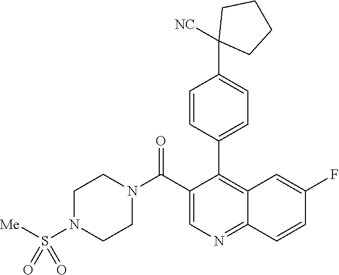 [SMILES: CS(=O)(=O)N1CCN(C(=O)c2cnc3ccc(F)cc3c2-c2ccc(C3(C#N)CCCC3)cc2)CC1] | 1-(4-(6-fluoro-3-(4- (methylsulfonyl)piperazine-1- carbonyl)quinolin-4- yl)phenyl)cyclopentane-1- carbonitrile |
| C-107 | 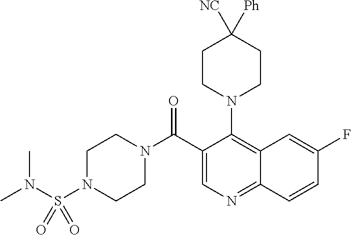 [SMILES: CN(C)S(=O)(=O)N1CCN(C(=O)c2cnc3ccc(F)cc3c2N2CCC(C#N)(c3ccccc3)CC2)CC1] | 4-(4-(4-cyano-4-phenylpiperidin-1- yl)-6-fluoroquinoline-3-carbonyl)- N,N-dimethylpiperazine-1- sulfonamide |
| C-108 | 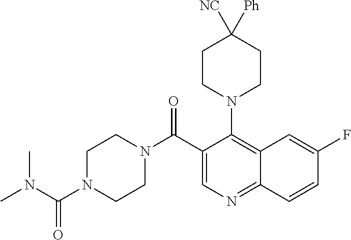 [SMILES: CN(C)C(=O)N1CCN(C(=O)c2cnc3ccc(F)cc3c2N2CCC(C#N)(c3ccccc3)CC2)CC1] | 4-(4-(4-cyano-4-phenylpiperidin-1- yl)-6-fluoroquinoline-3-carbonyl)- N,N-dimethylpiperazine-1- carboxamide |
| C-109 | 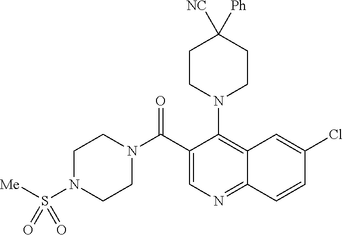 [SMILES: CS(=O)(=O)N1CCN(C(=O)c2cnc3ccc(Cl)cc3c2N2CCC(C#N)(c3ccccc3)CC2)CC1] | 1-(6-chloro-3-(4- (methylsulfonyl)piperazine-1- carbonyl)quinolin-4-yl)-4- phenylpiperidine-4-carbonitrile |
| C-110 | 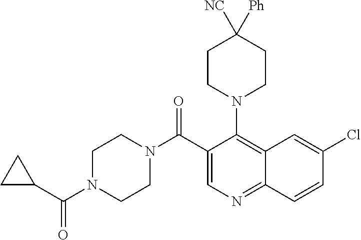 [SMILES: N#CC1(c2ccccc2)CCN(c2c(C(=O)N3CCN(C(=O)C4CC4)CC3)cnc3ccc(Cl)cc23)CC1] | 1-(6-chloro-3-(4- (cyclopropanecarbonyl)piperazine- 1-carbonyl)quinolin-4-yl)-4- phenylpiperidine-4-carbonitrile |
| C-111 | 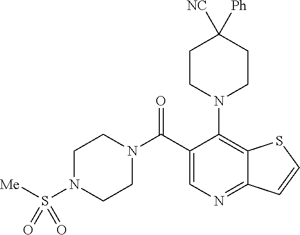 [SMILES: CS(=O)(=O)N1CCN(C(=O)c2cnc3ccsc3c2N2CCC(C#N)(c3ccccc3)CC2)CC1] | 1-(6-(4-(methylsulfonyl)piperazine- 1-carbonyl)thieno[3,2-b]pyridin-7- yl)-4-phenylpiperidine-4- carbonitrile |
| C-112 | 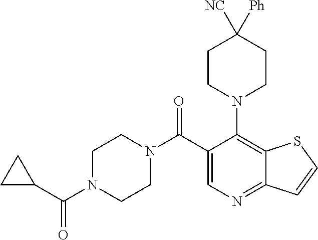 [SMILES: N#CC1(c2ccccc2)CCN(c2c(C(=O)N3CCN(C(=O)C4CC4)CC3)cnc3ccsc23)CC1] | 1-(6-(4- (cyclopropanecarbonyl)piperazine- 1-carbonyl)thieno[3,2-b]pyridin-7- yl)-4-phenylpiperidine-4- carbonitrile |
| C-113 | 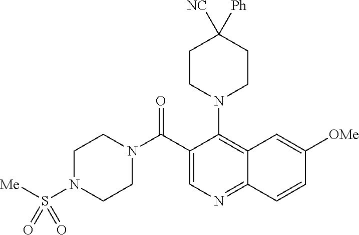 [SMILES: COc1ccc2ncc(C(=O)N3CCN(S(C)(=O)=O)CC3)c(N3CCC(C#N)(c4ccccc4)CC3)c2c1] | 1-(6-methoxy-3-(4- (methylsulfonyl)piperazine-1- carbonyl)quinolin-4-yl)-4- phenylpiperidine-4-carbonitrile |
| C-114 | 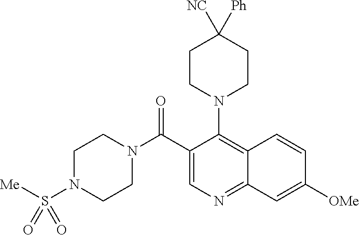 [SMILES: COc1ccc2c(N3CCC(C#N)(c4ccccc4)CC3)c(C(=O)N3CCN(S(C)(=O)=O)CC3)cnc2c1] | 1-(7-methoxy-3-(4- (methylsulfonyl)piperazine-1- carbonyl)quinolin-4-yl)-4- phenylpiperidine-4-carbonitrile |
| C-115 | 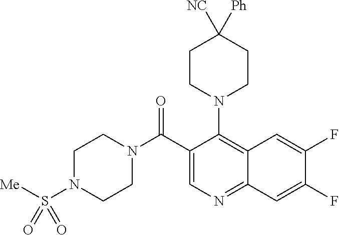 [SMILES: CS(=O)(=O)N1CCN(C(=O)c2cnc3cc(F)c(F)cc3c2N2CCC(C#N)(c3ccccc3)CC2)CC1] | 1-(6,7-difluoro-3-(4- (methylsulfonyl)piperazine-1- carbonyl)quinolin-4-yl)-4- phenylpiperidine-4-carbonitrile |
| C-116 | 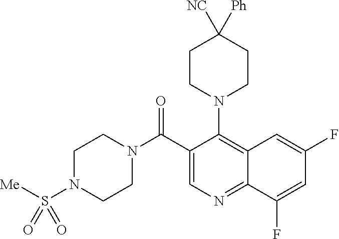 [SMILES: CS(=O)(=O)N1CCN(C(=O)c2cnc3c(F)cc(F)cc3c2N2CCC(C#N)(c3ccccc3)CC2)CC1] | 1-(6,8-difluoro-3-(4- (methylsulfonyl)piperazine-1- carbonyl)quinolin-4-yl)-4- phenylpiperidine-4-carbonitrile |
| C-117 | 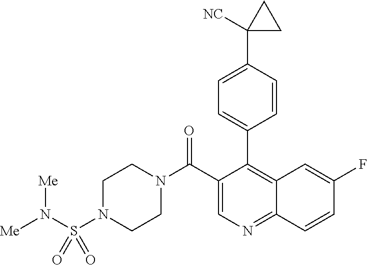 [SMILES: CN(C)S(=O)(=O)N1CCN(C(=O)c2cnc3ccc(F)cc3c2-c2ccc(C3(C#N)CC3)cc2)CC1] | 4-(4-(4-(1- cyanocyclopropyl)phenyl)-6- fluoroquinoline-3-carbonyl)-N,N- dimethylpiperazine-1-sulfonamide |
| C-118 | 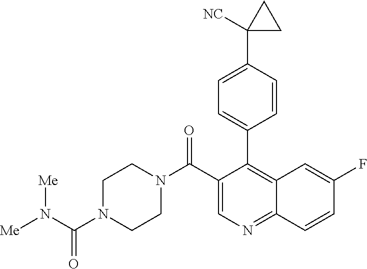 [SMILES: CN(C)C(=O)N1CCN(C(=O)c2cnc3ccc(F)cc3c2-c2ccc(C3(C#N)CC3)cc2)CC1] | 4-(4-(4-(1- cyanocyclopropyl)phenyl)-6- fluoroquinoline-3-carbonyl)-N,N- dimethylpiperazine-1-carboxamide |
| C-119 | 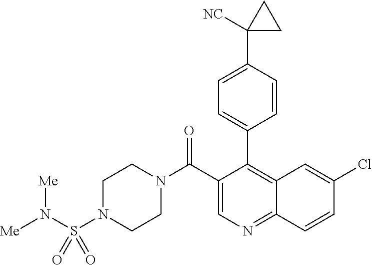 [SMILES: CN(C)S(=O)(=O)N1CCN(C(=O)c2cnc3ccc(Cl)cc3c2-c2ccc(C3(C#N)CC3)cc2)CC1] | 4-(6-chloro-4-(4-(1- cyanocyclopropyl)phenyl)quinoline- 3-carbonyl)-N,N- dimethylpiperazine-1-sulfonamide |
| C-120 | 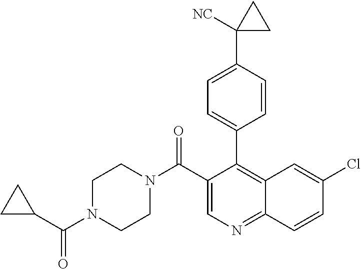 [SMILES: N#CC1(c2ccc(-c3c(C(=O)N4CCN(C(=O)C5CC5)CC4)cnc4ccc(Cl)cc34)cc2)CC1] | 1-(4-(6-chloro-3-(4- (cyclopropanecarbonyl)piperazine- 1-carbonyl)quinolin-4- yl)phenyl)cyclopropane-1- carbonitrile |
| C-121 | 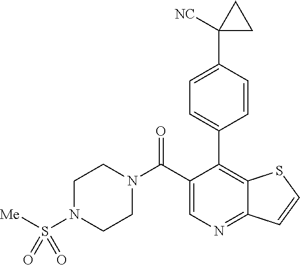 [SMILES: CS(=O)(=O)N1CCN(C(=O)c2cnc3ccsc3c2-c2ccc(C3(C#N)CC3)cc2)CC1] | 1-(4-(6-(4- (methylsulfonyl)piperazine-1- carbonyl)thieno[3,2-b]pyridin-7- yl)phenyl)cyclopropane-1- carbonitrile |
| C-122 | 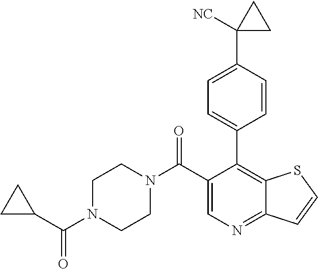 [SMILES: N#CC1(c2ccc(-c3c(C(=O)N4CCN(C(=O)C5CC5)CC4)cnc4ccsc34)cc2)CC1] | 1-(4-(6-(4- (cyclopropanecarbonyl)piperazine- 1-carbonyl)thieno[3,2-b]pyridin-7- yl)phenyl)cyclopropane-1- carbonitrile |
| C-123 | 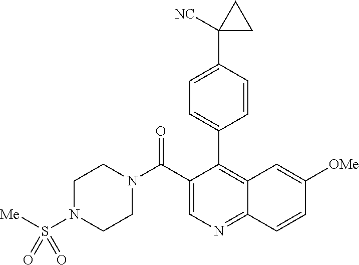 [SMILES: COc1ccc2ncc(C(=O)N3CCN(S(C)(=O)=O)CC3)c(-c3ccc(C4(C#N)CC4)cc3)c2c1] | 1-(4-(6-methoxy-3-(4- (methylsulfonyl)piperazine-1- carbonyl)quinolin-4- yl)phenyl)cyclopropane-1- carbonitrile |
| C-124 | 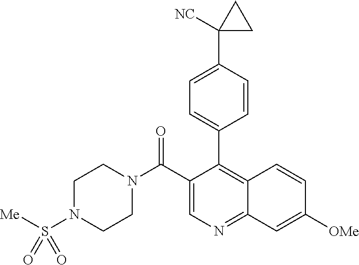 [SMILES: COc1ccc2c(-c3ccc(C4(C#N)CC4)cc3)c(C(=O)N3CCN(S(C)(=O)=O)CC3)cnc2c1] | 1-(4-(7-methoxy-3-(4- (methylsulfonyl)piperazine-1- carbonyl)quinolin-4- yl)phenyl)cyclopropane-1- carbonitrile |
| C-125 | 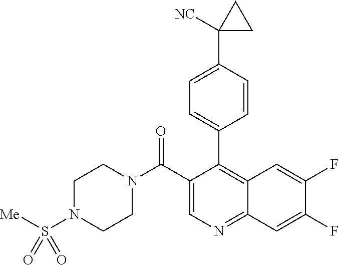 [SMILES: CS(=O)(=O)N1CCN(C(=O)c2cnc3cc(F)c(F)cc3c2-c2ccc(C3(C#N)CC3)cc2)CC1] | 1-(4-(6,7-difluoro-3-(4- (methylsulfonyl)piperazine-1- carbonyl)quinolin-4- yl)phenyl)cyclopropane-1- carbonitrile |
| C-126 | 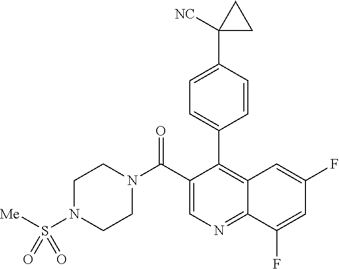 [SMILES: CS(=O)(=O)N1CCN(C(=O)c2cnc3c(F)cc(F)cc3c2-c2ccc(C3(C#N)CC3)cc2)CC1] | 1-(4-(6,8-difluoro-3-(4- (methylsulfonyl)piperazine-1- carbonyl)quinolin-4- yl)phenyl)cyclopropane-1- carbonitrile |
| C-9125 | 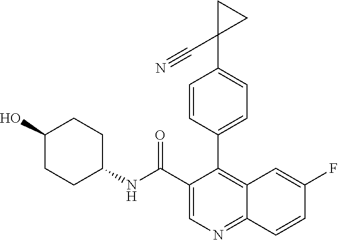 [SMILES: N#CC1(c2ccc(-c3c(C(=O)N[C@H]4CC[C@H](O)CC4)cnc4ccc(F)cc34)cc2)CC1] | 4-(4-(1-cyanocyclopropyl)phenyl)- 6-fluoro-N-((1r,4r)-4- hydroxycyclohexyl)quinoline-3- carboxamide |
| C-9163 | 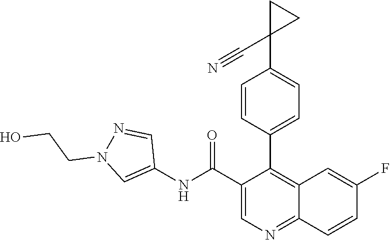 [SMILES: N#CC1(c2ccc(-c3c(C(=O)Nc4cnn(CCO)c4)cnc4ccc(F)cc34)cc2)CC1] | 4-(4-(1-cyanocyclopropyl)phenyl)- 6-fluoro-N-(1-(2-hydroxyethyl)-1H- pyrazol-4-yl)quinoline-3- carboxamide |
| C-9175 | 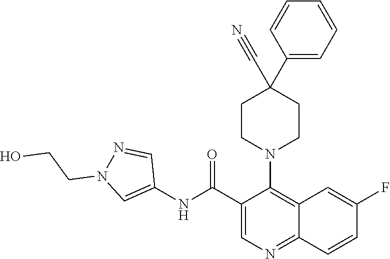 [SMILES: N#CC1(c2ccccc2)CCN(c2c(C(=O)Nc3cnn(CCO)c3)cnc3ccc(F)cc23)CC1] | 4-(4-cyano-4-phenylpiperidin-1-yl)- 6-fluoro-N-(1-(2-hydroxyethyl)-1H- pyrazol-4-yl)quinoline-3- carboxamide |

### Retinoic Acid Receptor Inhibitor or Retinoid X Receptor Inhibitor

In certain embodiments, the methods and/or compositions described herein include retinoic acid receptor inhibitors and/or retinoid X receptor inhibitors. Retinoic acid receptor inhibitors or retinoid X receptor inhibitors for use in the methods described herein may be chemically synthesized by methods known in the art, or may be purchased from commercial sources. In certain embodiments, the retinoic acid receptor inhibitor is selected from the group consisting of AGN 193109, BMS 195614, BMS 493 CD 2665, ER 50891, LE 135, LY 2955303, MM 11253 any salt or solvate thereof, and any combinations thereof (Table 2).

TABLE 2

| Compound | Structure | Name |
|---|---|---|
| AGN 193109 | 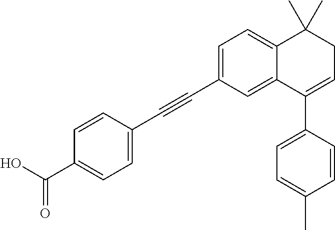 [SMILES: Cc1ccc(C2=CCC(C)(C)c3ccc(C#Cc4ccc(C(=O)O)cc4)cc32)cc1] | 4-[2-[5,6-Dihydro-5,5-dimethyl-8- (4-methylphenyl)-2- naphthalenyl]ethynyl]-benzoic acid |
| BMS 195614 | 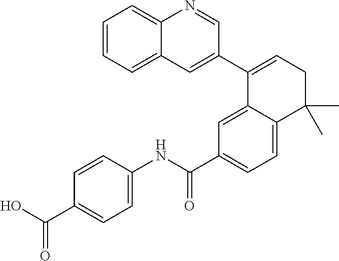 [SMILES: CC1(C)CC=C(c2cnc3ccccc3c2)c2cc(C(=O)Nc3ccc(C(=O)O)cc3)ccc21] | 4-(5,5-dimethyl-8-(quinolin-3-yl)- 5,6-dihydronaphthalene-2- carboxamido)benzoic acid |
| BMS 493 | 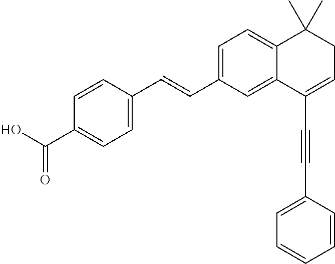 [SMILES: CC1(C)CC=C(C#Cc2ccccc2)c2cc(/C=C/c3ccc(C(=O)O)cc3)ccc21] | (E)-4-(2-(5,5-dimethyl-8- (phenylethynyl)-5,6- dihydronaphthalen-2- yl)vinyl)benzoic acid |
| CD 2665 | 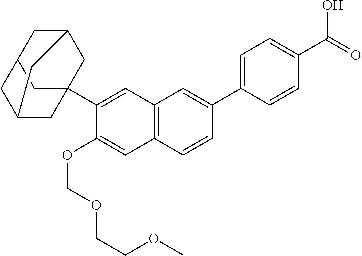 [SMILES: COCCOCOc1cc2ccc(-c3ccc(C(=O)O)cc3)cc2cc1C12CC3CC(CC(C3)C1)C2] | 4-(7-(adamantan-1-yl)-6-((2- methoxyethoxy)methoxy)naphthalen- 2-yl)benzoic acid |
| ER 50891 | 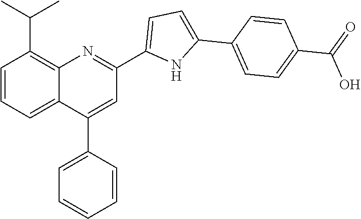 [SMILES: CC(C)c1cccc2c(-c3ccccc3)cc(-c3ccc(-c4ccc(C(=O)O)cc4)[nH]3)nc12] | 4-(5-(8-isopropyl-4- phenylquinolin-2-yl)-1H-pyrrol-2- yl)benzoic acid |
| LE 135 | 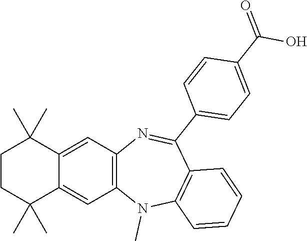 [SMILES: CN1c2cc3c(cc2N=C(c2ccc(C(=O)O)cc2)c2ccccc21)C(C)(C)CCC3(C)C] | 4-(5,7,7,10,10-pentamethyl- 7,8,9,10-tetrahydro-5H- benzo[e]naphtho[2,3- b][1,4]diazepin-13-yl)benzoic acid |
| LY 2955303 | 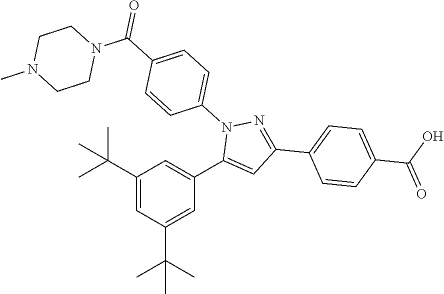 [SMILES: CN1CCN(C(=O)c2ccc(-n3nc(-c4ccc(C(=O)O)cc4)cc3-c3cc(C(C)(C)C)cc(C(C)(C)C)c3)cc2)CC1] | 4-(5-(3,5-di-tert-butylphenyl)-1-(4- (4-methylpiperazine-1- carbonyl)phenyl)-1H-pyrazol-3- yl)benzoic acid |
| MM 11253 | 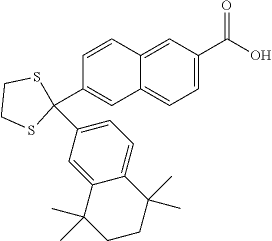 [SMILES: CC1(C)CCC(C)(C)c2cc(C3(c4ccc5cc(C(=O)O)ccc5c4)SCCS3)ccc21] | 6-(2-(5,5,8,8-tetramethyl-5,6,7,8- tetrahydronaphthalen-2-yl)-1,3- dithiolan-2-yl)-2-naphthoic acid |

AGN 193109 is a PAN-RAR antagonist. BMS 195614 is a selective RAR-alpha antagonist. BMS 493 is a PAN-RAR antagonist/inverse agonist. CD 2665 is a selective RAR-beta and RAR-gamma antagonist. ER 50891 is a selective RAR-alpha antagonist. LE 135 is a selective RAR-beta antagonist. LY 2955303 is a selective RAR-gamma antagonist. MM 11253 is a selective RAR-gamma antagonist.

In some embodiments, the retinoid X receptor inhibitor is selected from the group consisting of HX 531, PA 452, and UVI 3003, any salt or solvate thereof, and any combinations thereof (Table 3). HX 531 is a PAN-RXR antagonist. PA 452 is a PAN-RXR antagonist. UVI 3003 is a PAN-RXR antagonist.

TABLE 3

| Compound | Structure | Name |
|---|---|---|
| HX 531 | 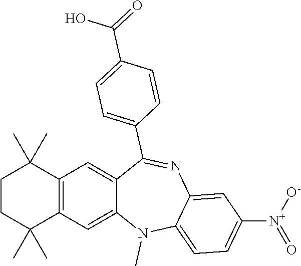 [SMILES: CN1c2ccc([N+](=O)[O-])cc2N=C(c2ccc(C(=O)O)cc2)c2cc3c(cc21)C(C)(C)CCC3(C)C] | 4-(5,7,7,10,10-pentamethyl-2-nitro- 7,8,9,10-tetrahydro-5H- benzo[b]naphtho[2,3- c][1,4]diazepin-12-yl)benzoic acid |
| PA 452 | 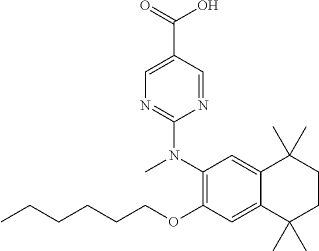 [SMILES: CCCCCCOc1cc2c(cc1N(C)c1ncc(C(=O)O)cn1)C(C)(C)CCC2(C)C] | 2-((3-(hexyloxy)-5,5,8,8- tetramethyl-5,6,7,8- tetrahydronaphthalen-2- yl)(methyl)amino)pyrimidine-5- carboxylic acid |
| UVI 3003 | 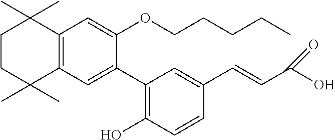 [SMILES: CCCCCOc1cc2c(cc1-c1cc(/C=C/C(=O)O)ccc1O)C(C)(C)CCC2(C)C] | (E)-3-(4-hydroxy-3-(5,5,8,8- tetramethyl-3-(pentyloxy)-5,6,7,8- tetrahydronaphthalen-2- yl)phenyl)acrylic acid |

### Immunostimulators

### Immune Checkpoint Inhibitors

As described herein, the term “immune checkpoint inhibitor” includes any compound or composition that is capable of inhibiting immune checkpoint molecules that are regulators of the immune system (e.g., stimulate or inhibit immune system activity). For example, some checkpoint inhibitors block inhibitory checkpoint molecules, thereby stimulating immune system function, such as stimulation of T-cell activity against cancer cells. A non-limiting example of a checkpoint inhibitor is a PD-L1 inhibitor.

As described herein, the term “PD-L1 inhibitor” includes any compound that is capable of inhibiting the expression and/or function of the protein Programmed Death-Ligand 1 (PD-L1) either directly or indirectly. PD-L1, also known as cluster of differentiation 274 (CD274) or B7 homolog 1 (B7-H1), is a type 1 transmembrane protein that plays a major role in suppressing the adaptive arm of immune system. PD-L1 binds to its receptor, the inhibitory checkpoint molecule PD-1 (which is found on activated T cells, B cells, and myeloid cells) so as to modulate activation or inhibition of the adaptive arm of immune system. In certain embodiments, the PD-L1 inhibitor (i.e. anti-PD-L1 antibody) inhibits the expression and/or function of PD-L1 by at least 5%, at least 10%, at least 20%, at least 50%, at least 75%, or at least 90%.

Reported PD-L1 inhibitors include, but are not limited to, compounds recited in one of the following patent application publications: US 2018/0057455; US 2018/0057486; WO 2017/106634; WO 2018/026971; WO 2018/045142; WO 2018/118848; WO 2018/119221; WO 2018/119236; WO 2018/119266; WO 2018/119286; WO 2018/121560; WO 2019/076343; WO 2019/087214; and are incorporated herein in their entirety by reference.

In certain embodiments, the immune checkpoint inhibitor is selected from the group consisting of an anti-PD1 antibody, an anti-PD-L1 antibody, an anti-CTLA4 antibody, any fragment thereof, and any combinations thereof.

### Chimeric Antigen Receptor (CAR) T-Cells

As described elsewhere herein, the term “chimeric antigen receptor (CAR) T-cell” refers to T-cells that have been genetically engineered to provide an artificial T-cell receptor for use in immunotherapy. The artificial T-cell receptor, comprising a chimeric antigen receptor (CAR) expressed on the surface of the T-cell, provides both antigen-binding and T-cell activating functions, thereby permitting activation of a host immune response upon antigen binding, through any of a number of mechanisms (e.g. T-cell proliferation and monocyte differentiation). The antigen-binding domain of the CAR is selected on the basis of specific proteins identified as overexpressed on the surface of the tumor cell in the subject. Preferably, the antigen overexpressed on the surface of the tumor is unique to the cancerous cells and is not present to a substantial extent on the surface of healthy cells.

In certain embodiments, T-cells are harvested autologously from the subject. In other embodiments, donor T-cells may be used. Harvested T-cells are subjected to genetic engineering to express the desired CAR on the cellular surface. Further, the CAR T-cells are administered to the subject to illicit a targeted immune response. In certain embodiments, the CAR T-cells are administered intravenously. CAR T-cell therapy comprises the process described herein (i.e. harvesting T-cells, modifying T-cells, administering CAR T-cells) for the treatment of a disease in a subject (e.g. hepatocellular carcinoma).

### TCR-Transgenic T Cells

As described elsewhere herein, the term “TCR-transgenic T cell” refers to T-cells that have been genetically engineered to provide a natural T-cell receptor for use in immunotherapy. T cells recognize antigens via T cell receptors (TCRs) comprised of α (alpha) and β (beta) chains. However, αβ-TCRs can recognize and respond to antigens only when the antigen is presented as an 8-11 amino acid peptide fragment by a major histocompatibility complex (MHC) on the target cell. CD8 T cells recognize antigens presented by MHC class I (expressed by all nucleated cells) while CD4 T cells recognize those presented by MHC class II (expressed by specialized antigen-presenting cells). Tumoricidal T cells are usually cytotoxic (or killer) CD8 T cells that recognize tumor-associated antigens presented by MHC class I on the surface of tumor cells. TCR-transgenic approach is based on enforcing expression of a ‘synthetic’ TCR with defined specificity for a tumor antigen-MHCI complex in T cells. The advantage of this approach is the ability to target any protein (nuclear, cytosolic, or membrane bound) expressed by the tumor. On the flip side, a major barrier is the highly polymorphic nature of MHC and the requirement to select patients based on the co-expression of the relevant MHC-I molecule and the target antigen. Hence, a synthetic TCR for an antigen on a particular MHC allele will not recognize it when presented by another MHC allele, thereby restricting the benefits of the tgTCR to patients expressing a specific MHC allele.

### Adoptive Transfer of Autologous Anti-Tumor T Cells (Ex Vivo Expanded T-Cells)

As described elsewhere, adoptive transfer of autologous anti-tumor T cells refers to a non-genetically engineered approach whereby tumor-reactive T cells are identified and isolated from the patient's own tumor, ex vivo expansion of these T cells, and subsequent infusion of these T cells back into the same patient. An advantage of this approach is enhanced survival and minimal rejection of the infused T cells as these are autologous.

### Bispecific T Cell Engagers (BiTE)

As described herein, a bispecific T cell engager (BiTE) is a synthetic protein that simultaneously binds a T cell and a target antigen on the tumor cell independent of MHC-TCR interaction. In certain embodiments, this enforces contact between a T cell and the tumor cell. A significant advantage of BiTEs is that they can be used ‘off the shelf’ as it is not specific to a particular patient, tumor, or T cell sub-type. The major limitation is that not all T cells that are brought into tumor cell proximity by this approach are activated or capable of tumor cell killing.

### Compounds

The present disclosure provides a compound of formula (I), or a salt, solvate, prodrug, stereoisomer, tautomer, or isotopologue thereof:

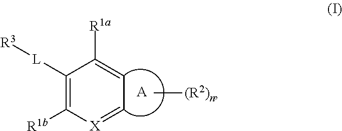

wherein:
R^{1a} is selected from the group consisting of optionally substituted C₂-C₈ heterocyclyl, optionally substituted phenyl, and optionally substituted C₅-C₈ cycloalkenyl,
   wherein each optional substituent in R^{1a} is independently selected from the group consisting of C₁-C₆ alkyl, C₃-C₆ cycloalkyl, C₁-C₆ alkoxy, optionally substituted phenyl, optionally substituted C₂-C₈ heterocyclyl, halogen, OH, N(R^{a})(R^{b}), NO₂, CN, C(═O)R^{a}, C(═O)OR^{a}, C(═O)N(R^{a})(R^{b}), S(═O)₂R^{a}, S(═O)₂OR^{a}, S(═O)₂N(R^{a})(R^{b}), OC(═O)R^{a}, N(R^{a})=S(═O)(R^{b})(R^{c}), S(═O)(=NR^{a})R^{b}, and N(R^{a})C(═O)R^{b},
      wherein each optional substituent is optionally substituted with at least one substituent selected from the group consisting of C₁-C₃ alkyl, C₁-C₃ alkoxy, halogen, CN, and NO₂, and
   wherein two vicinal or geminal optional substituents in R^{1a} may combine with the atoms to which they are bound to form a C₂-C₈ heterocyclyl or C₃-C₈ cycloalkyl;
R^{1b} and R^{1c}, if present, are each independently selected from the group consisting of H, C₁-C₆ alkyl, C₁-C₆ alkoxy, halogen, OH, N(R^{a})(R^{b}), NO₂, and CN;
each occurrence of R² is independently selected from the group consisting of C₁-C₆ alkyl, C₁-C₆ alkoxy, C₁-C₃ haloalkoxy, C₁-C₆ hydroxyalkyl, halogen, NO₂, and CN;
R³ is selected from the group consisting of optionally substituted C₂-C₈ heterocyclyl, optionally substituted phenyl, N(R^{a})(optionally substituted C₃-C₈ cycloalkyl), and N(R^{a})(optionally substituted C₂-C₈ heterocyclyl),
   wherein each optional substituent in R³ is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)OR^{a}, C(═O)N(R^{a})(R^{b}), S(═O)₂R^{a}, S(═O)₂OR^{a}, S(═O)₂N(R^{a})(R^{b}), OC(═O)R^{a}, and N(R)C(═O)R^{b};
A is selected from the group consisting of optionally substituted C₆-C₁₀ aryl and optionally substituted C₂-C₈ heterocyclyl,
   wherein each optional substituent in A is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)OR^{a}, C(═O)N(R^{a})(R^{b}), S(═O)₂R^{a}, S(═O)₂OR^{a}, S(═O)₂N(R^{a})(R^{b}), OC(═O)R^{a}, and N(R^{a})C(═O)R^{b};
L is selected from the group consisting of —CH₂—, —C(═O)—, and —S(═O)₂—;
X is selected from the group consisting of N and CR^{1c};
n is an integer selected from the group consisting of 0, 1, 2, 3, and 4; and
each occurrence of R^{a}, R^{b}, and R^{c} is independently selected from the group consisting of H, C₁-C₆ alkyl, C₂-C₆ alkenyl, C₂-C₆ alkynyl, C₃-C₈ cycloalkyl, and C₂-C₈ heterocyclyl; and
(ii) a compound selected from the group consisting of:
   1-benzylindoline-2,3-dione;
   ethyl 2-((4-oxo-3-(3-(pyrrolidin-1-yl)propyl)-3,4-dihydrobenzo[4,5]thieno[3,2-d]pyrimidin-2-yl)thio)acetate;
   2-((2-(sec-butyl)-3-oxo-2,3-dihydroimidazo[1,2-c]quinazolin-5-yl)thio)-N-(o-tolyl)butanamide; and
   8-((4-(cyclopropanecarbonyl)piperazin-1-yl)methyl)-7-isopentyl-1,3-dimethyl-3,7-dihydro-1H-purine-2,6-dione;
   or a salt, solvate, prodrug, stereoisomer, tautomer, or isotopologue thereof.

In certain embodiments, the C₂-C₈ heterocyclyl in R^{1a} is thiophen-2-yl. In certain embodiments, the C₂-C₈ heterocyclyl in R^{1a} is thiophen-3-yl.

In certain embodiments, the optionally substituted C₆-C₁₀ aryl in A is optionally substituted phenyl. In certain embodiments, the optionally substituted C₂-C₈ heterocyclyl is optionally substituted C₂-C₅ heterocyclyl.

In certain embodiments, the RALDH1 inhibitor is a compound of formula (I).

In certain embodiments, the compound of formula (I) is selected from the group consisting of:

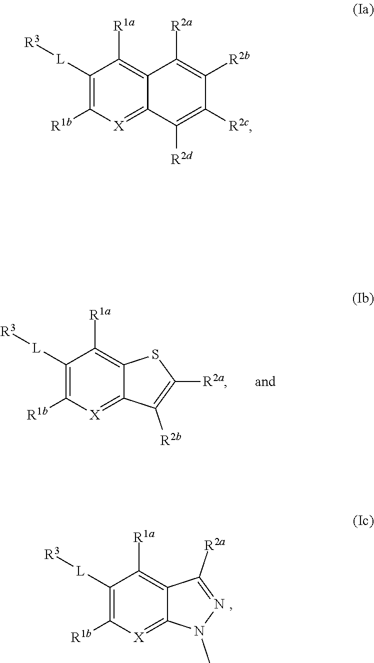

wherein:
R^{2a}, R^{2b}, R^{2c}, and R^{2d}, if present, are each independently selected from the group consisting of H, C₁-C₆ alkyl, C₁-C₆ alkoxy, and halogen.

In certain embodiments, R^{2a}, R^{2b}, R^{2c}, and R^{2d}, if present, are each independently selected from the group consisting of H, Me, OMe, F, and Cl.

In certain embodiments, X is N.

In certain embodiments, R^{1a} is selected from the group consisting of:

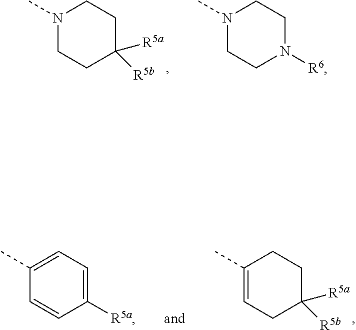

wherein:
R^{5a} and R^{5b}, if present, are each independently selected from the group consisting of C₁-C₆ alkyl, C₁-C₆ alkoxy, C₃-C₆ cycloalkyl, phenyl, thiophen-2-yl, thiophen-3-yl, and CN,
   wherein each substituent in R^{5a} and R^{5b} is optionally substituted with at CN, and
   wherein R^{5a} and R^{5b}, if present, may combine with the atoms to which they are bound to form a C₂-C₆ heterocyclyl or C₃-C₆ cycloalkyl; and
R⁶ is S(═O)₂R^{a}.

In certain embodiments, R^{5a} is Me. In certain embodiments, R^{5a} is t-tBu. In certain embodiments, R^{5a} is 1-cyanocyclopropyl. In certain embodiments, R^{5a} is 1-cyanocyclopentyl. In certain embodiments, R^{5a} is phenyl. In certain embodiments, R^{5a} is CN. In certain embodiments, R^{5a} is 1-cyanocyclobutyl. In certain embodiments, R^{5a} is 1-cyanocyclohexyl. In certain embodiments, R^{5a} is thiophen-2-yl. In certain embodiments, R^{5a} is thiophen-3-yl.

In certain embodiments, R^{5b} is Me. In certain embodiments, R^{5b} is t-tBu. In certain embodiments, R^{5b} is 1-cyanocyclopropyl. In certain embodiments, R^{5b} is 1-cyanocyclopentyl. In certain embodiments, R^{5b} is phenyl. In certain embodiments, R^{5b} is CN. In certain embodiments, R^{5b} is 1-cyanocyclobutyl. In certain embodiments, R^{5b} is 1-cyanocyclohexyl. In certain embodiments, R^{5b} is thiophen-2-yl. In certain embodiments, R^{1b} is thiophen-3-yl.

In certain embodiments, R⁶ is ethenylsulfonyl.

In certain embodiments, R^{1a} is

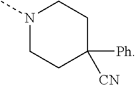

In certain embodiments, R^{1a} is

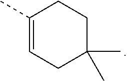

In certain embodiments, R^{1a} is

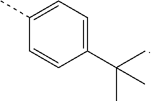

In certain embodiments, R^{1a} is

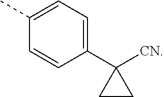

In certain embodiments, R^{1a} is

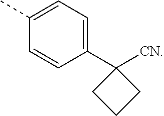

In certain embodiments, R^{1a} is

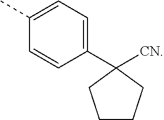

In certain embodiments, R^{1a} is

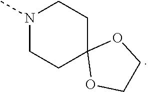

In certain embodiments, R^{1a} is

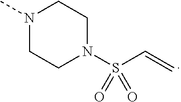

In certain embodiments, R^{1a} is

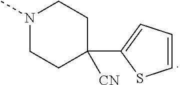

In certain embodiments, R^{1b} is H.

In certain embodiments, R³ is selected from the group consisting of

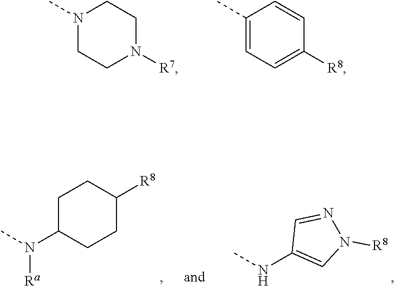

wherein:
R⁷ is selected from the group consisting of C(═O)R^{a} and S(═O)₂R^{a}; and
R⁸ is selected from the group consisting of C₁-C₆ alkoxy, C₁-C₆ hydroxyalkyl, and OH.

In certain embodiments, R⁷ is cyclopropylcarbonyl. In certain embodiments, R⁷ is methylsulfonyl. In certain embodiments, R⁷ is dimethylaminosulfonyl. In certain embodiments, R⁷ is dimethylaminocarbonyl.

In certain embodiments, R⁸ is methoxy. In certain embodiments, R⁸ is 2-hydroxyethyl.

In certain embodiments, R⁸ is OH.

In certain embodiments, R³ is

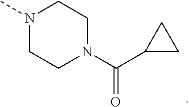

In certain embodiments, R³ is

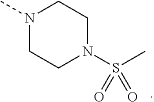

In certain embodiments, R³ is

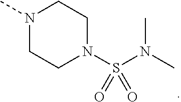

In certain embodiments, R³ is

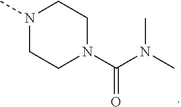

In certain embodiments, R³ is

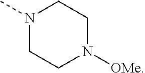

In certain embodiments, R³ is

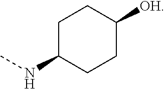

In certain embodiments, R³ is

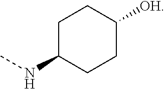

In certain embodiments, R³ is

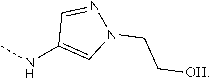

In certain embodiments, the compound of formula (I) is selected from the group consisting of:
8-(6-methoxy-3-((4-methoxyphenyl)sulfonyl)56uinoline-4-yl)-1,4-dioxa-8-azaspiro[4.5]decane;
1-(3-(4-(cyclopropanecarbonyl)piperazine-1-carbonyl)-6-fluoroquinolin-4-yl)-4-phenylpiperidine-4-carbonitrile;
(4-(cyclopropanecarbonyl)piperazin-1-yl)(4-(4,4-dimethylcyclohex-1-en-1-yl)-6-fluoroquinolin-3-yl)methanone;
(4-(4-(tert-butyl)phenyl)-6-fluoroquinolin-3-yl)(4-(cyclopropanecarbonyl)piperazin-1-yl)methanone;
1-(4-(3-(4-(cyclopropanecarbonyl)piperazine-1-carbonyl)-6-fluoroquinolin-4-yl)phenyl)cyclopropanecarbonitrile;
1-(6-fluoro-3-(4-(methylsulfonyl)piperazine-1-carbonyl)57uinoline-4-yl)-4-phenylpiperidine-4-carbonitrile;
(4-(4,4-dimethylcyclohex-1-en-1-yl)-6-fluoroquinolin-3-yl)(4-(methylsulfonyl)piperazin-1-yl)methanone;
(4-(4-(tert-butyl)phenyl)-6-fluoroquinolin-3-yl)(4-(methylsulfonyl)piperazin-1-yl)methanone;
1-(4-(6-fluoro-3-(4-(methylsulfonyl)piperazine-1-carbonyl)57uinoline-4-yl)phenyl)cyclopropane-1-carbonitrile;
4-(4-cyano-4-phenylpiperidin-1-yl)-6-fluoro-N-((1r,4r)-4-hydroxycyclohexyl)quinoline-3-carboxamide;
(6-fluoro-4-(4-(vinylsulfonyl)piperazin-1-yl)57uinoline-3-yl)(4-(methylsulfonyl)piperazin-1-yl)methanone;
1-(4-(6-fluoro-3-(4-(methylsulfonyl)piperazine-1-carbonyl)57uinoline-4-yl)phenyl)57uinoline57e-1-carbonitrile;
1-(4-(6-fluoro-3-(4-(methylsulfonyl)piperazine-1-carbonyl)57uinoline-4-yl)phenyl)cyclopentane-1-carbonitrile;
4-(4-(4-cyano-4-phenylpiperidin-1-yl)-6-fluoroquinoline-3-carbonyl)-N,N-dimethylpiperazine-1-sulfonamide;
4-(4-(4-cyano-4-phenylpiperidin-1-yl)-6-fluoroquinoline-3-carbonyl)-N,N-dimethylpiperazine-1-carboxamide;
1-(6-chloro-3-(4-(methylsulfonyl)piperazine-1-carbonyl)57uinoline-4-yl)-4-phenylpiperidine-4-carbonitrile;
1-(6-chloro-3-(4-(cyclopropanecarbonyl)piperazine-1-carbonyl)57uinoline-4-yl)-4-phenylpiperidine-4-carbonitrile;
1-(6-(4-(methylsulfonyl)piperazine-1-carbonyl)thieno[3,2-b]58uinolin-7-yl)-4-phenylpiperidine-4-carbonitrile;
1-(6-(4-(cyclopropanecarbonyl)piperazine-1-carbonyl)thieno[3,2-b]58uinolin-7-yl)-4-phenylpiperidine-4-carbonitrile;
1-(6-methoxy-3-(4-(methylsulfonyl)piperazine-1-carbonyl)58uinoline-4-yl)-4-phenylpiperidine-4-carbonitrile;
1-(7-methoxy-3-(4-(methylsulfonyl)piperazine-1-carbonyl)58uinoline-4-yl)-4-phenylpiperidine-4-carbonitrile;
1-(6,7-difluoro-3-(4-(methylsulfonyl)piperazine-1-carbonyl)58uinoline-4-yl)-4-phenylpiperidine-4-carbonitrile;
1-(6,8-difluoro-3-(4-(methylsulfonyl)piperazine-1-carbonyl)58uinoline-4-yl)-4-phenylpiperidine-4-carbonitrile;
4-(4-(4-(1-cyanocyclopropyl)phenyl)-6-fluoroquinoline-3-carbonyl)-N,N-dimethylpiperazine-1-sulfonamide;
4-(4-(4-(1-cyanocyclopropyl)phenyl)-6-fluoroquinoline-3-carbonyl)-N,N-dimethylpiperazine-1-carboxamide;
4-(6-chloro-4-(4-(1-cyanocyclopropyl)phenyl)quinoline-3-carbonyl)-N,N-dimethylpiperazine-1-sulfonamide;
1-(4-(6-chloro-3-(4-(cyclopropanecarbonyl)piperazine-1-carbonyl)58uinoline-4-yl)phenyl)cyclopropane-1-carbonitrile;
1-(4-(6-(4-(methylsulfonyl)piperazine-1-carbonyl)thieno[3,2-b]58uinolin-7-yl)phenyl)cyclopropane-1-carbonitrile;
1-(4-(6-(4-(cyclopropanecarbonyl)piperazine-1-carbonyl)thieno[3,2-b]58uinolin-7-yl)phenyl)cyclopropane-1-carbonitrile;
1-(4-(6-methoxy-3-(4-(methylsulfonyl)piperazine-1-carbonyl)58uinoline-4-yl)phenyl)cyclopropane-1-carbonitrile;
1-(4-(7-methoxy-3-(4-(methylsulfonyl)piperazine-1-carbonyl)58uinoline-4-yl)phenyl)cyclopropane-1-carbonitrile;
1-(4-(6,7-difluoro-3-(4-(methylsulfonyl)piperazine-1-carbonyl)58uinoline-4-yl)phenyl)cyclopropane-1-carbonitrile;
1-(4-(6,8-difluoro-3-(4-(methylsulfonyl)piperazine-1-carbonyl)59uinoline-4-yl)phenyl)cyclopropane-1-carbonitrile;
4-(4-(1-cyanocyclopropyl)phenyl)-6-fluoro-N-((1r,4r)-4-hydroxycyclohexyl)quinoline-3-carboxamide;
4-(4-(1-cyanocyclopropyl)phenyl)-6-fluoro-N-(1-(2-hydroxyethyl)-1H-pyrazol-4-yl)quinoline-3-carboxamide; and
4-(4-cyano-4-phenylpiperidin-1-yl)-6-fluoro-N-(1-(2-hydroxyethyl)-1H-pyrazol-4-yl)quinoline-3-carboxamide.

### Pharmaceutical Compositions and Formulations

The present disclosure further provides a pharmaceutical composition comprising:
(a) at least one immunostimulator;
(b) a pharmaceutically acceptable carrier; and
I a retinaldehyde dehydrogenase 1 (RALDH1) inhibitor, wherein the RALDH1 inhibitor is selected from the group consisting of:
(i) a compound of formula (I), or a salt, solvate, prodrug, stereoisomer, tautomer, or isotopologue thereof:

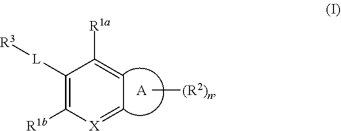

wherein:
R^{1a} is selected from the group consisting of optionally substituted C₂-C₈ heterocyclyl, optionally substituted phenyl, and optionally substituted C₅-C₈ cycloalkenyl,
   wherein each optional substituent in R^{1a} is independently selected from the group consisting of C₁-C₆ alkyl, C₃-C₆ cycloalkyl, C₁-C₆ alkoxy, optionally substituted phenyl, optionally substituted C₂-C₈, halogen, OH, N(R^{a})(R^{b}), NO₂, CN, C(═O)R^{a}, C(═O)OR^{a}, C(═O)N(R^{a})(R^{b}), S(═O)₂R^{a}, S(═O)₂OR^{a}, S(═O)₂N(R^{a})(R^{b}), OC(═O)R^{a}, N(R^{a})=S(═O)(R)(R^{c}), S(═O)(=NR^{a})R, and N(R^{a})C(═O)R^{b},
      wherein each optional substituent is optionally substituted with at least one substituent selected from the group consisting of C₁-C₃ alkyl, C₁-C₃ alkoxy, halogen, CN, and NO₂, and
   wherein two vicinal or geminal optional substituents in R^{1a} may combine with the atoms to which they are bound to form a C₂-C₈ heterocyclyl or C₃-C₈ cycloalkyl;
R^{1b} and R^{1c}, if present, are each independently selected from the group consisting of H, C₁-C₆ alkyl, C₁-C₆ alkoxy, halogen, OH, N(R^{a})(R^{b}), NO₂, and CN;
each occurrence of R² is independently selected from the group consisting of C₁-C₆ alkyl, C₁-C₆ alkoxy, C₁-C₃ haloalkoxy, C₁-C₆ hydroxyalkyl, halogen, NO₂, and CN;
R³ is selected from the group consisting of optionally substituted C₂-C₈ heterocyclyl, optionally substituted phenyl, N(R^{a})(optionally substituted C₃-C₈ cycloalkyl), and N(R^{a})(optionally substituted C₂-C₈ heterocyclyl),
   wherein each optional substituent in R³ is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)OR^{a}, C(═O)N(R^{a})(R^{b}), S(═O)₂R^{a}, S(═O)₂OR^{a}, S(═O)₂N(R^{a})(R^{b}), OC(═O)R^{a}, and N(R^{a})C(═O)R^{b};
A is selected from the group consisting of optionally substituted C₆-C₁₀ aryl and optionally substituted C₂-C₈ heterocyclyl,
   wherein each optional substituent in A is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)OR^{a}, C(═O)N(R^{a})(R^{b}), S(═O)₂R^{a}, S(═O)₂OR^{a}, S(═O)₂N(R^{a})(R^{b}), OC(═O)R^{a}, and N(R^{a})C(═O)R^{b};
L is selected from the group consisting of —CH₂—, —C(═O)—, and —S(═O)₂—;
X is selected from the group consisting of N and CR^{1c};
n is an integer selected from the group consisting of 0, 1, 2, 3, and 4; and
each occurrence of R^{a}, R^{b}, and R^{c} is independently selected from the group consisting of H, C₁-C₆ alkyl, C₂-C₆ alkenyl, C₂-C₆ alkynyl, C₃-C₈ cycloalkyl, and C₂-C₈ heterocyclyl; and
(ii) a compound selected from the group consisting of:
   1-benzylindoline-2,3-dione;
   ethyl 2-((4-oxo-3-(3-(pyrrolidin-1-yl)propyl)-3,4-dihydrobenzo[4,5]thieno[3,2-d]pyrimidin-2-yl)thio)acetate;
   2-((2-(sec-butyl)-3-oxo-2,3-dihydroimidazo[1,2-c]quinazolin-5-yl)thio)-N-(o-tolyl)butanamide; and
   8-((4-(cyclopropanecarbonyl)piperazin-1-yl)methyl)-7-isopentyl-1,3-dimethyl-3,7-dihydro-1H-purine-2,6-dione;
   or a salt, solvate, prodrug, stereoisomer, tautomer, or isotopologue thereof.

In certain embodiments, the C₂-C₈ heterocyclyl in R^{1a} is thiophen-2-yl. In certain embodiments, the C₂-C₈ heterocyclyl in R^{1a} is thiophen-3-yl.

In certain embodiments, the optionally substituted C₆-C₁₀ aryl in A is optionally substituted phenyl. In certain embodiments, the optionally substituted C₂-C₈ heterocyclyl is optionally substituted C₂-C₅ heterocyclyl.

In certain embodiments, the RALDH1 inhibitor is a compound of formula (I).

In certain embodiments, the compound of formula (I) is selected from the group consisting of:

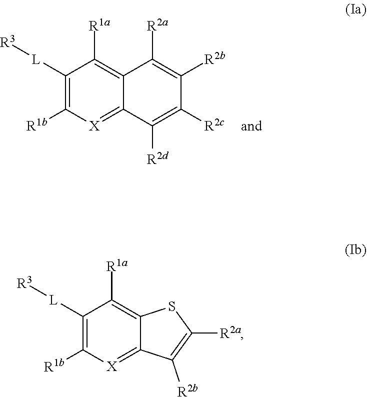

wherein:
R^{2a}, R^{2b}, R^{2c}, and R^{2d}, if present, are each independently selected from the group consisting of H, C₁-C₆ alkyl, C₁-C₆ alkoxy, and halogen.

In certain embodiments, R^{2a}, R^{2b}, R^{2c}, and R^{2d}, if present, are each independently selected from the group consisting of H, Me, OMe, F, and Cl.

In certain embodiments, X is N.

In certain embodiments, R^{1a} is selected from the group consisting of:

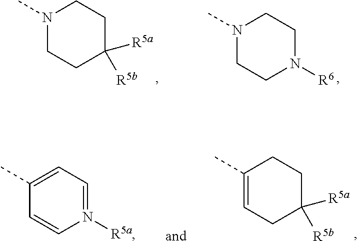

wherein:
R^{5a} and R^{5b}, if present, are each independently selected from the group consisting of C₁-C₆ alkyl, C₁-C₆ alkoxy, C₃-C₆ cycloalkyl, phenyl, thiophen-2-yl, thiophen-3-yl, and CN,
   wherein each substituent in R^{5a} and R^{5b} is optionally substituted with at CN, and
   wherein R^{5a} and R^{5b}, if present, may combine with the atoms to which they are bound to form a C₂-C₆ heterocyclyl or C₃-C₆ cycloalkyl; and
R⁶ is S(═O)₂R^{a}.

In certain embodiments, R^{5a} is Me. In certain embodiments, R^{5a} is t-tBu. In certain embodiments, R^{5a} is 1-cyanocyclopropyl. In certain embodiments, R^{5a} is 1-cyanocyclopentyl. In certain embodiments, R^{5a} is phenyl. In certain embodiments, R^{5a} is CN. In certain embodiments, R^{5a} is 1-cyanocyclobutyl. In certain embodiments, R^{5a} is 1-cyanocyclohexyl. In certain embodiments, R^{5a} is thiophen-2-yl. In certain embodiments, R^{5a} is thiophen-3-yl.

In certain embodiments, R^{5b} is Me. In certain embodiments, R^{5b} is t-tBu. In certain embodiments, R^{5b} is 1-cyanocyclopropyl. In certain embodiments, R^{5b} is 1-cyanocyclopentyl. In certain embodiments, R^{5b} is phenyl. In certain embodiments, R^{5b} is CN. In certain embodiments, R^{5b} is 1-cyanocyclobutyl. In certain embodiments, R^{5b} is 1-cyanocyclohexyl. In certain embodiments, R^{5b} is thiophen-2-yl. In certain embodiments, R^{5b} is thiophen-3-yl.

In certain embodiments, R⁶ is ethenylsulfonyl.

In certain embodiments, R^{1a} is

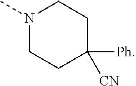

In certain embodiments, R^{1a} is

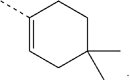

In certain embodiments, R^{1a} is

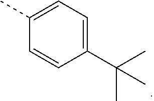

In certain embodiments, R^{1a} is

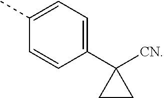

In certain embodiments, R^{1a} is

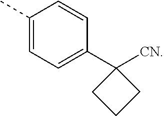

In certain embodiments, R^{1a} is

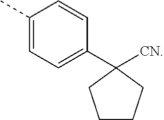

In certain embodiments, R^{1a} is

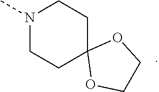

In certain embodiments, R^{1a} is

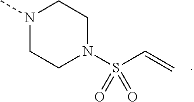

In certain embodiments, R^{1a} is

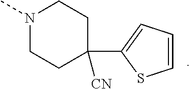

In certain embodiments, R^{1b} is H.

In certain embodiments, R³ is selected from the group consisting of:

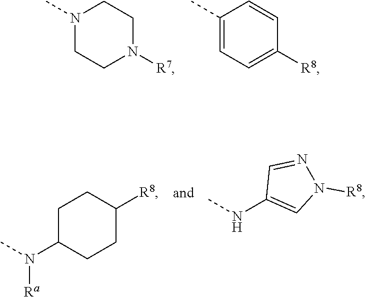

wherein:
R⁷ is selected from the group consisting of C(═O)R^{a} and S(═O)₂R^{a}; and
R⁸ is selected from the group consisting of C₁-C₆ alkoxy, C₁-C₆ hydroxyalkyl, and OH.

In certain embodiments, R⁷ is cyclopropylcarbonyl. In certain embodiments, R⁷ is methylsulfonyl. In certain embodiments, R⁷ is dimethylaminosulfonyl. In certain embodiments, R⁷ is dimethylaminocarbonyl.

In certain embodiments, R⁸ is methoxy. In certain embodiments, R⁸ is 2-hydroxyethyl. In certain embodiments, R⁸ is OH.

In certain embodiments, R³ is

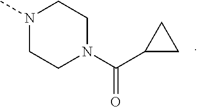

In certain embodiments, R³ is

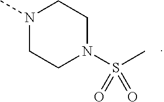

In certain embodiments, R³ is

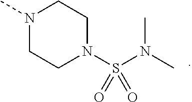

In certain embodiments, R³ is

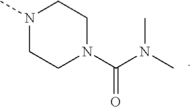

In certain embodiments, R³ is

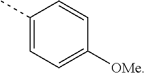

In certain embodiments, R³ is

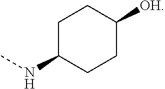

In certain embodiments, R³ is

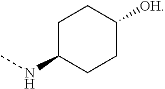

In certain embodiments, R³ is

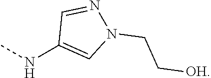

In certain embodiments, the compound of formula (I) is selected from the group consisting of:
8-(6-methoxy-3-((4-methoxyphenyl)sulfonyl)quinolin-4-yl)-1,4-dioxa-8-azaspiro[4.5]decane;
1-(3-(4-(cyclopropanecarbonyl)piperazine-1-carbonyl)-6-fluoroquinolin-4-yl)-4-phenylpiperidine-4-carbonitrile;
(4-(cyclopropanecarbonyl)piperazin-1-yl)(4-(4,4-dimethylcyclohex-1-en-1-yl)-6-fluoroquinolin-3-yl)methanone;
(4-(4-(tert-butyl)phenyl)-6-fluoroquinolin-3-yl)(4-(cyclopropanecarbonyl)piperazin-1-yl)methanone;
1-(4-(3-(4-(cyclopropanecarbonyl)piperazine-1-carbonyl)-6-fluoroquinolin-4-yl)phenyl)cyclopropanecarbonitrile;
1-(6-fluoro-3-(4-(methylsulfonyl)piperazine-1-carbonyl)quinolin-4-yl)-4-phenylpiperidine-4-carbonitrile;
(4-(4,4-dimethylcyclohex-1-en-1-yl)-6-fluoroquinolin-3-yl)(4-(methylsulfonyl)piperazin-1-yl)methanone;
(4-(4-(tert-butyl)phenyl)-6-fluoroquinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclopropane-1-carbonitrile;
4-(4-cyano-4-phenylpiperidin-1-yl)-6-fluoro-N-((1r,4r)-4-hydroxycyclohexyl)quinoline-3-carboxamide;
(6-fluoro-4-(4-(vinylsulfonyl)piperazin-1-yl)quinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclobutane-1-carbonitrile;
1-(4-(6-fluoro-3-(4-(methylsulfonyl)piperazine-1-carbonyl)quinolin-4-yl)phenyl)cyclopentane-1-carbonitrile;
4-(4-(4-cyano-4-phenylpiperidin-1-yl)-6-fluoroquinoline-3-carbonyl)-N,N-dimethylpiperazine-1-sulfonamide;
4-(4-(4-cyano-4-phenylpiperidin-1-yl)-6-fluoroquinoline-3-carbonyl)-N,N-dimethylpiperazine-1-carboxamide;
1-(6-chloro-3-(4-(methylsulfonyl)piperazine-1-carbonyl)quinolin-4-yl)-4-phenylpiperidine-4-carbonitrile;
1-(6-chloro-3-(4-(cyclopropanecarbonyl)piperazine-1-carbonyl)quinolin-4-yl)-4-phenylpiperidine-4-carbonitrile;
1-(6-(4-(methylsulfonyl)piperazine-1-carbonyl)thieno[3,2-b]pyridin-7-yl)-4-phenylpiperidine-4-carbonitrile;
1-(6-(4-(cyclopropanecarbonyl)piperazine-1-carbonyl)thieno[3,2-b]pyridin-7-yl)-4-phenylpiperidine-4-carbonitrile;
1-(6-methoxy-3-(4-(methylsulfonyl)piperazine-1-carbonyl)quinolin-4-yl)-4-phenylpiperidine-4-carbonitrile;
1-(7-methoxy-3-(4-(methylsulfonyl)piperazine-1-carbonyl)quinolin-4-yl)-4-phenylpiperidine-4-carbonitrile;
1-(6,7-difluoro-3-(4-(methylsulfonyl)piperazine-1-carbonyl)quinolin-4-yl)-4-phenylpiperidine-4-carbonitrile;
1-(6,8-difluoro-3-(4-(methylsulfonyl)piperazine-1-carbonyl)quinolin-4-yl)-4-phenylpiperidine-4-carbonitrile;
4-(4-(4-(1-cyanocyclopropyl)phenyl)-6-fluoroquinoline-3-carbonyl)-N,N-dimethylpiperazine-1-sulfonamide;
4-(4-(4-(1-cyanocyclopropyl)phenyl)-6-fluoroquinoline-3-carbonyl)-N,N-dimethylpiperazine-1-carboxamide;
4-(6-chloro-4-(4-(1-cyanocyclopropyl)phenyl)quinoline-3-carbonyl)-N,N-dimethylpiperazine-1-sulfonamide;
1-(4-(6-chloro-3-(4-(cyclopropanecarbonyl)piperazine-1-carbonyl)quinolin-4-yl)phenyl)cyclopropane-1-carbonitrile;
1-(4-(6-(4-(methylsulfonyl)piperazine-1-carbonyl)thieno[3,2-b]pyridin-7-yl)phenyl)cyclopropane-1-carbonitrile;
1-(4-(6-(4-(cyclopropanecarbonyl)piperazine-1-carbonyl)thieno[3,2-b]pyridin-7-yl)phenyl)cyclopropane-1-carbonitrile;
1-(4-(6-methoxy-3-(4-(methylsulfonyl)piperazine-1-carbonyl)quinolin-4-yl)phenyl)cyclopropane-1-carbonitrile;
1-(4-(7-methoxy-3-(4-(methylsulfonyl)piperazine-1-carbonyl)quinolin-4-yl)phenyl)cyclopropane-1-carbonitrile;
1-(4-(6,7-difluoro-3-(4-(methylsulfonyl)piperazine-1-carbonyl)quinolin-4-yl)phenyl)cyclopropane-1-carbonitrile;
1-(4-(6,8-difluoro-3-(4-(methylsulfonyl)piperazine-1-carbonyl)quinolin-4-yl)phenyl)cyclopropane-1-carbonitrile;
4-(4-(1-cyanocyclopropyl)phenyl)-6-fluoro-N-((1r,4r)-4-hydroxycyclohexyl)quinoline-3-carboxamide;
4-(4-(1-cyanocyclopropyl)phenyl)-6-fluoro-N-(1-(2-hydroxyethyl)-1H-pyrazol-4-yl)quinoline-3-carboxamide; and
4-(4-cyano-4-phenylpiperidin-1-yl)-6-fluoro-N-(1-(2-hydroxyethyl)-1H-pyrazol-4-yl)quinoline-3-carboxamide.

In certain embodiments, the immunostimulator is at least one selected from the group consisting of an immune checkpoint inhibitor, chimeric antigen receptor (CAR) T-cells, T-cells engineered to express specific TCR targeted tumor antigens (TCR-transgenic), ex vivo expanded T-cells, and bispecific T-cell engagers (BiTE).

In certain embodiments, the immunostimulator is an immune checkpoint inhibitor.

In certain embodiments, the immune checkpoint inhibitor is selected from the group consisting of an anti-PD1 antibody, an anti-PD-L1 antibody, an anti-CTLA4 antibody, any fragment thereof, and any combinations thereof.

In certain embodiments, the immunostimulator is CAR T-cells.

In certain embodiments, the pharmaceutically acceptable carrier is suitable for intravenous administration.

In certain embodiments, the at least one immunostimulator comprises an immune checkpoint inhibitor and CAR T-cells.

In certain embodiments, the pharmaceutical composition further comprises at least one selected from the group consisting of a retinoic acid receptor (RAR) inhibitor and a retinoid X receptor (RXR) inhibitor.

In certain embodiments, the RAR inhibitor is selected from the group consisting of AGN 193109, BMS 195614, BMS 493, CD 2665, ER 50891, LE 135, LY 2955303, MM 11253, any salt or solvate thereof, and any combinations thereof.

In certain embodiments, the RXR inhibitor is selected from the group consisting of HX 531, PA 452, and UVI 3003, any salt or solvate thereof, and any combinations thereof.

The invention provides pharmaceutical compositions comprising at least one compound of the invention or a salt or solvate thereof, which are useful to practice methods of the invention. Such a pharmaceutical composition may consist of at least one compound of the invention or a salt or solvate thereof, in a form suitable for administration to a subject, or the pharmaceutical composition may comprise at least one compound of the invention or a salt or solvate thereof, and one or more pharmaceutically acceptable carriers, one or more additional ingredients, or some combination of these. At least one compound of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. One such preservative is a combination of about 0.5% to 2.0% benzyl alcohol and 0.05% to 0.5% sorbic acid.

The composition may include an antioxidant and a chelating agent which inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physician taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically-or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the compositions and/or formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXAMPLES

Various embodiments of the present application can be better understood by reference to the following Examples which are offered by way of illustration. The scope of the present application is not limited to the Examples given herein.

### Materials and Methods

### Animals

Wild type C57BL/6 mice were purchased from Jackson Laboratories (Cat #: 000664). Immuodeficient NU/J mice were purchased from Jackson Laboratories (Cat #: 002019). dnRAR^{flox} mice were made available. LysM^{Cre} mice were purchased from Jackson Laboratories (Cat #: 004781).

### Generation of RALDH1-KO Mice

Aldh1A1 knockout mice were created through the University of Pennsylvania's CRISPR/Cas9 Mouse targeting core facility. Two CRISPR RNAs were designed that encompassed a ~36 kilobase region within the mouse Aldh1A1 gene (Gencode Gene: ENSMUSG00000053279.8. Position: mm10 chr19:20,492,715-20,643,465).

Aldh1a1_5p_crRNA:
(SEQ ID NO: 1)
CTGAGTTGGACCCTATATGG
Aldh1a1_3p_crRNA:
(SEQ ID NO: 2)
GAGAATGTGTTGGTGCCTCG

A mix of pure Cas9 mRNA and the guide RNAs were injected into single cell zygotes of C57BL/6 background mice. Founders were identified by a PCR-based genotyping protocol designed to detect the gene deletion. Founders were then bred to wild type C57BL/6 mice to ‘fix’ the allele. Heterozygous pups were identified by the aforementioned PCR-based genotyping and bred to each other to generate RALDH1-KO mice. Additional qRT-PCR assays were performed (described under ‘RNA isolation and qPCR analysis for gene expression’ section) to confirm absence of the Aldh1a1 transcripts.

### PCR Primers for RALDH1-KO Genotyping (Sequence 5′-3)

WT_9753
(SEQ ID NO: 3)
CAACCCTGAGCAAATCCTCCAC
WT_9754
(SEQ ID NO: 4)
GACAGATTGAGAGCAGTGTTTACCC
Aldh1a1-KO_F1:
(SEQ ID NO: 5)
TGATATGTCCCAGGAAGATGAA
Aldh1a1-KO_R2:
(SEQ ID NO: 6)
GGACCGAGCACTTGCCTA

### PCR Conditions

5 mins at 94 degrees Celsius. 35 cycles of: (1) 30 seconds at 94 degrees Celsius, (2) 30 seconds at 58 degrees Celsius, and (3) 30 seconds at 72 degrees Celsius. Final extension of 7 minutes at 72 degrees Celsius followed by storage at 4 degrees Celsius. 248 base pairs band in knockouts and 665 base pairs band in WT detected by conventional gel electrophoresis.

### Tumor Cells

The details of the cell lines and culture media are provided herein. Upon receipt, the cell lines were first expanded (two passages), authenticated, and then frozen into aliquots for storage. The frozen stocks were thawed and expanded (average three passages) prior to experiments and discarded upon completion of individual experiments. When needed, the frozen stocks were re-expanded (average of two passages), authenticated, and stored as frozen aliquots of additional stock. Huh1 cell line was provided. The cells were obtained in 2022 and authenticated based on their morphology and growth characteristics in cell culture as well as the histology of the tumors formed upon transplantation into mice. Huh7, SNU449, SNU398, HEP3B, PLC, HEPA 1-6, HEP55, and AL458A were obtained in 2021 and authenticated based on their morphology and growth characteristics in cell culture as well as the histology of the tumors formed upon transplantation into mice. Fibrosarcoma (FS) cell lines have been described in the literature. Tumor cell lines were cultured in DMEM (ThermosFisher, Cat #10567014) with 10% FBS (GeminiBio, Cat #100-500) 1% Pen/Strep (ThermoFisher Scientific, Cat #15140122) and 2 mM glutamine (ThermoFisher Scientific, 25030081). All cells were confirmed to be negative for mycoplasma contamination as assessed by MycoAlert Mycoplasma Detection Kit (Lonza, Cat #LT07).

### Human Samples

A pathologist identified Human FFPE (formalin-fixed, paraffin-embedded) samples of normal liver, normal kidney, HCC, GIST, and CRC from patients (de-identified) who underwent therapeutic surgical resection for diagnostic or therapeutic purposes. 56 archived tissue blocks were selected and de-identified prior to sectioning and IHC. Normal donor human monocytes and T cells were collected by the Human Immunology Core (HIC) at the University of Pennsylvania and purchased from HIC.

### Implantation of Tumor Cells, Tumor Growth Measurements, and Survival Analyses

Cultured Huh7, Huh1, Hepa 1-6, Hep55, and FS (as indicated in figure legends) tumor cells were detached using 0.25% trypsin (Gibco, catalog #25200056), washed once with 1×PBS, and counted before implantation. 3~6×10⁶ tumor cells were implanted subcutaneously (s.c.) into shaved flanks of recipient mice. Tumor dimensions were measured using a caliper starting at the day indicated in the accompanying figure legend and every two to three days thereafter; volume was calculated by using formula Length*Width²/2. Tumor volumes of 2,000 mm³, tumor length of 2 cm or tumor ulceration were used as endpoints for survival analyses.

### Flow Cytometry of Tissue Samples

Tissue samples Murine tumors of the type indicated in the corresponding figure legends were harvested and single-cell suspensions were generated by digestion with collagenase B and Dnase I (both Sigma Aldrich) for 45 minutes at 37° C. and filtration through 70 M cell strainers. Red blood cells were lysed using RBC Lysis Buffer (Biolegend). Samples were incubated for 20 minutes on ice with anti-mouse CD16/32 Fc Block (BD Biosciences), and subsequently stained on ice with primary-fluorophore conjugated antibodies for identification of cell populations by flow cytometry. Flow cytometry was performed on an LSRII Flow Cytometer (BD Biosciences) and analyzed using FlowJo software, Treestar, version 10.8.1).

### In Vitro Tumor Cell Proliferation Assay

1×10⁴~2×10⁵ of Huh7, Huh1, Hepa 1-6, and Hep55 tumor cells were plated in triplicate in 48-well plates or 6-well plates. Viable cell numbers were counted every day for three to four days.

### In Vitro Treatments

Cultures of tumor cells or primary monocyte-derived cells were treated with C86, C91, C99, BMS 493 (Tocris, 3509) or Win 18446 (Tocris, 4736) at a time and concentration indicated in corresponding figure legends.

### AldeRed Assay

The AldeRed assay (EMD Millipore) was performed according to manufacturer's instructions to identify cells with ALDH activity. In brief, single-cell suspensions of Huh7, Huh1, SNU449, SNU398, HEP3B, PLC, Hepa 1-6, Hep55, and AL458A cultured cells or single-cell suspension generated from enzymatic digestion of tumors generated from transplantation of these cells in mice (as indicated in figure legends) were incubated with a fluorescent and non-toxic ALDH substrate (AldeRed 588-A); the fluorescent product accumulates in cells proportional to their ALDH activity. The amount of fluorescence produced is measured by flow cytometry. The ALDH inhibitor diethylaminobenzaldehyde (DEAB, provided with the AldeRed assay kit) was used as a negative control for background fluorescence assessment.

### Cell Sorting

GFP expressing Huh7 or Hep55 cells from CRISPR Knockout experiments were isolated using a FACS Jazz cell sorter. Cells were identified and isolated based on GFP positivity alone on the cell sorter.

LC-MS for all-Trans Retinoic Acid (ATRA)

For measurement of all-trans retinoic acid (ATRA), cultured cells were detached using trypsin, centrifuged, and the cell pellets stored at −80. ATRA was extracted from the frozen cell pellets and quantified using liquid chromatography tandem mass spectrometry (LC-MS) as previously described in the literature.

### Isolation of Mouse Bone Marrow Monocytes

Monocytes were isolated from bone marrow of C57BL/6 mice using the Mouse BM Monocyte Isolation Kit (Miltenyi Biotec) according to manufacturer's instructions. Purity of monocyte was assessed by flow cytometry using CD11b, Ly6C, and Ly6G.

### Intratumoral Monocyte Transfer

Monocytes were isolated (as described above) from mouse bone marrow of C57BL/6 mice and counted. Subsequently, 1×10⁶ monocytes were resuspended in 50 μL 1×PBS and injected directly into established tumors at Day 12 post tumor-cell implantation. Tumors were harvested at specified time points and analyzed by flow cytometry. For human monocytes, primary human monocytes were purchased from the HIC core facility at the University of Pennsylvania and 1×10⁶ monocytes were injected intratumorally as described elsewhere herein. This method of intratumoral monocyte transfer is described in the literature.

### In Vitro and Ex Vivo Mouse and Human Monocyte Differentiation Assays

Mouse monocytes were isolated from bone marrow as described herein and then cultured in RPMI 1640 (ThermoFisher Scientific, Cat #11875085) with 10% fetal bovine serum (GeminiBio, Cat #100-500). GM-CSF (20 ng/mL, peprotech 315-03) and IL4 (20 ng/mL, peprotech 214-14) were added for dendritic cell differentiation while M-CSF (20 ng/mL, peprotech 315-02) was added for macrophage differentiation. Human monocytes were purchased from the human immunology core facility at the University of Pennsylvania and cultured in RPMI 1640 (ThermoFisher Scientific, Cat #11875085) with 10% fetal bovine serum (GeminiBio, Cat #100-500). GM-CSF (50 ng/mL, peprotech 300-03) and IL4 (50 ng/mL, peprotech 200-04) were added to cultures for dendritic cell differentiation while or M-CSF (50 ng/mL, peprotech 300-25) was added for macrophage differentiation. RA (200 nM; Sigma Aldrich), C86 (100 nM) or tumor conditioned medium (TCM) was added at specified time points for indicated differentiation assays. Cellular identity and function of differentiated monocytes was assessed by flow cytometry and quantitative PCR (qPCR).

### Depletion of Cells In Vivo

To deplete T cells, 200 μg of mouse CD3-specific antibody (clone 17A2) was administered i.p. starting three days before tumor implantation and repeated every three to four days until mouse sacrifice. To deplete macrophages, 200 μL of clodronate liposome (CloLipo) or PBS-liposome (CtrlLipo) (both Liposoma) was administered i.p. starting three days prior to tumor implantation and repeated every four days until mouse sacrifice. Macrophage depletion efficacy in spleen and within tumors was confirmed by flow cytometry using canonical macrophage marker F4/80.

### Drug Treatment In Vivo

Compound-86, 97 and 99 powder was dissolved in 20% HPβCD saline (2-Hydroxypropyl)-1-cyclodextrin). Drug was administered i.p. or p.o. starting when tumor volume reached 50-150 mm³ and repeated every day.

200 μg of PD1-specific monoclonal blocking antibody (clone RMP 1-14) was administered i.p. starting when tumor volume reached 50-150 mm³ and repeated every two days.

### Pharmacokinetic (PK) and Toxicological Studies

The PK studies were done at NIH or a commercial CRO—Pharmacon using their in-house standard protocol. For C91, IV and PO, performed at NIH using CD-1 mice; formulation: 20% HPbCD in saline. For C86, IV and PO, performed at Pharmaron using CD-1 mice; formulation: 20% HPbCD in saline. For C99, IV and PO, performed at NIH using CD-1 mice; formulation: 20% HPbCD in saline. IP PK of C86 at 10 mpk and 30 mpk, performed at NIH using C57BL/6J mice; formulation: 60% PEG400 in DI water. Chow PK of C86, performed at Pharmaron using CD-1 mice at 10, 30, and 60 mpk, which the dose concentration in chow is 0.05, 0.15, 0.3 mg/g, respectively, based on the calculation of 5 μg food consumption/mouse/day.

Pharmacokinetic (PK) studies at the NIH was performed by the DMPK group. Male CD1 or C57BL/6J mice between 6 and 8 weeks old and weighing approximately 20 to 30 grams were dosed with compound 86, compound 91, and compound 99 at 2 mg/kg (IV), 10 mg/kg (PO), and 10 or 30 mg/kg (IP). The compounds were formulated using a 20% hydroxypropyl-beta-cyclodextrin (HPbCD) solution in saline, was made on the day of dosing or directly prior to dosing. Each treatment group consisted of three mice, and plasma was collected at 5 minutes, 15 minutes, 30 minutes, 1 hour, 2 hours, 4 hours, 8 hours, 12 hours, and 24 hours post-dose for IV administration and at 15 minutes, 30 minutes, 1 hour, 2 hours, 4 hours, 8 hours, 12 hours, and 24 hours post-dose for PO administration. Approximately 0.025 mL of blood was collected from the dorsal metatarsal vein at each time point. The collected blood samples were then transferred into plastic microcentrifuge tubes containing heparin sodium as an anticoagulant. Samples were then centrifuged at 4000 g for 5 minutes at 4° C. to obtain plasma. Plasma samples were then stored in polypropylene tubes, quickly frozen, and kept at −75° C. until analyzed by LC/MS/MS. The following pharmacokinetic parameters were measured: Terminal half-life (T½), Concentration at immediately after injection (C0), Maximum concentration (Cmax), Time to reach max concentration (Tmax), Clearance (CL), Volume of distribution (Vd), Area under the curve (AUClast), and bioavailability (% F). Animals were also monitored during the in-life phase by once daily cage side observations; any adverse clinical signs were noted as part of the PK report.

Serum toxicological assays were performed by IDEXX Bioanalytics (Standard Tox Panel 62794). Briefly, peripheral blood was collected in regular Eppendorff tubes by tail snips following an approved protocol. Serum was prepared by letting the blood coagulate and inspected to confirm absence of hemolysis. The serum samples were stored in −80° C. until shipment to IDEXX.

Hematological studies, including complete blood count, were performed at IDEXX Bioanalytics. Briefly, peripheral blood was collected in heparin coated microhematocrit capillary tubes (VWR, 15401-560) by tail snips and stored at 4 degrees overnight before shipment to IDEXX.

RNA Isolation and qPCR Analysis for Gene Expression

Total RNA from mouse tissue samples and in vitro cultured cells was isolated using GenElute Mammalian Total RNA Miniprep Kit (Sigma) following the manufacturer's protocol. Total RNA from human FFPE samples was isolated by using Quick-RNA FFPE Miniprep Kit (Zymo Research) using the manufacturer's protocol. 1000 ng of RNA was used for reverse transcription using High Capacity RNA to cDNA Kit (Life Technologies). The cDNA product was 10× diluted. 2.5 μL of this cDNA was used for qPCR for each sample. Three or more replicates were used for each reaction. Target gene expression was normalized to appropriate housekeeping gene indicated in the legends of figures showing RT-qPCR data. Expression fold was calculated as 2\hat{ }−(CT target gene−CT housekeeping gene). Quantitative PCRs were run on a Viia 7 real time PCR system (ThermoFisher). The following target and housekeeping genes were measured using the commercially available TaqMan probes (ThermoFisher):

Mouse Hprt (Mm03024075_m1), Mouse Irf4 (Mm00516431_m1), Mouse Zbtb46 (Mm00511327_m1), Mouse Mafb (Mm00627481_s1), Mouse Aldh1a1 (Mm00657317_m1), Mouse Aldh1a2 (Mm00501306_m1), Mouse Aldh1a3 (Mm00474049_m1), Human HPRT (4333768F), Human IRF4 (Hs01056533_m1), Human ZBTB46 (Hs01008168_m1), Human MAFB (Hs00271378_s1), Human ALDH1A1 (Hs00946916_m1), Human ALDH1A2 (Hs00180254_m1), and Human ALDH1A3 (Hs00167476_m1.

### Western Blotting

1×10⁷ Cells were harvested and washed with PBS for three times. 50 μL RIPA Lysis Buffer (Thermo Scientific, Catalog number: 89901) containing proteinase inhibitor (Thermo Scientific, Catalog number: 78442) was added to the cell pellets and thoroughly mixed. Cell lysates were kept on ice for 30 min and centrifuge at 14000×g for 15 min. The supernatant was collected and protein concentration measured using BCA Protein Assay kit (Thermo Scientific, 23227). Mixed 30 μg cell lysate for each sample with loading buffer (Thermo Scientific, Catalog number: NP0007) to make a final volume of 20 μL and incubate at 95° C. for 5 min. Loaded samples on a pre-cast 4-15% SDS polyacrylamide gel (Cat. #4561084, BIO-RAD), and ran at 120V (constant voltage) for 40 to 60 minutes until the dye reached the bottom the gel. Transferred sample from the gel to the PVDF membrane in Tris-Glycine transfer buffer at 100 V for 1.5 hour at constant current (not to exceed 0.4 A). The PVDF membrane were taken out from the blotting cassette and rinsed with TBST (10 mM Tris-HCl, pH 8.0, 150 mM NaCl, 0.05% Tween 20) for 5 min at room temperature for 3 times. Non-specific binding on the membrane was blocked with freshly prepared 5% nonfat dried milk (Labscientific, Cat #M0841) for 1 hour on a shaker at room temperature for 1 h. Aldh1a1 rabbit polyclone primary antibody (Invitrogen, cat #PA5-32127) and GAPDH (14C10) rabbit mAb (Cell Signaling Technology, cat #2118S) were diluted with 5% BSA at the ratio of 1:1000 and incubated with the PVDF membrane at 4° C. overnight. Membrane was washed three times for 5 min each with TBST, and incubated with HRP-conjugated secondary antibody (Cell Signaling Technology, cat #7074) for 1 h at room temperature. The membrane was washed three times for 5 minutes each with TBST, and incubate with ECL substrate (PerkinElmer, cat #NEL104001EA) for 1 min. The image was taken by ChemiDoc imaging system (BioRad).

### Establishment of CRISPR-Mediated Gene Deletion Tumor Cell Lines

Non-viral delivery of Cas9-RNPs was previously described. In brief, crRNA and tracrRNA (both Integrated DNA Technologies) were mixed at equimolar concentrations and were annealed by heating at 95° C. for 5 min followed directly by hybridization for 15 min at room temperature. The annealed crRNA/XT-tracrRNA duplexes were mixed with Cas9 at a 3:1 molar ratio and were complexed by incubation at room temperature for ≥20 min. Nucleofection of Cas9-RNPs along with a GFP expressing plasmid vector (to identify cells undergoing successful nucleofection) was performed using Nucleofector™ 2 (Lonza). For the Huh7-RALDH1 knockout cell line, GFP positive cells were sorted by FACS Jazz and loss of RALDH1 expression was confirmed by western blot. For the Hep55-RALDH1 knockout cell line, the GFP positive cells were sorted by FACS Jazz and single cell clones were established from sorted cells. Loss of ALDH1A1 was confirmed in individual clones with Sanger sequencing performed at the University of Pennsylvania core facility and western blots performed in-house. The GFP expressing plasmid used in aforementioned nucleofection were supplied as a part of the Lonza nucleofection kit.

### Computational Analyses of RNA Sequencing (RNA-Seq) Data of Human Tumors

To compare RALDH isozyme expressions in HCCs vs. other human tumors in the TCGA dataset, the cBioPortal website interface was used for gene expression query. To examine RALDH isozyme expression between different HCC molecular subtypes, raw sequence counts were downloaded for 371 primary tumor samples in TCGA-LIHC from the Genomic Data Commons Data Portal, and filtered them, retaining the 183 samples that were included in iClusters 1-3 as described in the literature. On a local workstation, several Bioconductor packages in R were used for subsequent steps. The count data was annotated with biomaRt. Principal component analysis (PCA) and plots were generated with PCAtools. Normalizations and statistical analyses were done with DESeq2. Exploratory GSEA pathway analysis was done with fgsea against the hallmark pathway set from the Molecular Signatures Database (MsigDB), using the DESeq2 statistic as a ranking metric. Clustering was performed with the degPatterns function from the DEGreport package.

To examine the expression of RALDH isozymes in single cell RNA sequencing data of human HCC, a previously published and publicly available dataset was utilized. ALDH1A1, ALDH1A2, and ALDH1A3 were used as gene query terms using all samples in the dataset for the scatter plot output.

### Homology Modeling

To build a homology model MOE software with default settings was used. The software MOE (Molecular Operating Environment) is a suite of different software tools developed by Chemical Computing Group Inc. The homology modeling algorithm within MOE comprises of the following steps. First, Initial Partial Geometry Specification: an initial partial geometry for each target sequence is copied from regions of one or more template chains. Where residue identity is conserved between the target sequence and its template, all heavy-atom coordinates are copied; otherwise, only backbone coordinates are copied. Second, Insertions and Deletions for correction of no assigned backbone coordinates. They are modeled from fragments of high-resolution chains from the Protein Data Bank which superpose well onto anchor residues on either side of the insertion area. Third, Loop Selection and Sidechain Packing: after the indel data collection is complete, a set of independent models is created. Loops are modeled first, in random order. For each loop, a contact energy function analyzes the list of candidates collected in the segment searching stage, taking into account all atoms already modeled and any atoms specified by the user as belonging to the model environment (e.g., a ligand bound to the template, or structural water molecules). These energies are then used to make a Boltzmann-weighted choice from the candidates, the coordinates of which are then copied to the model. Once all of the loops have been chosen, the side chains are modeled. Sidechain data is assembled from an extensive rotamer library generated by systematic clustering of conformations from rotamer library. A deterministic procedure based on Unary Quadratic Optimization is then run to select an optimal packing. After all of the backbone segment and sidechain conformations have been chosen for an intermediate model, hydrogens are added to complete valence requirements and the model is submitted to a series of minimizations designed to first relieve any serious steric strains, and then to prepare the model to be scored. It is then written to the output database, along with a number of quality assessment measurements which can flag any serious geometric problems. The fourth stage is Final Model Selection and Refinement. The final model is based on the best-scoring intermediate model. The final model is based on the best-scoring intermediate model. In this study the electrostatic solvation energy was used, calculated used a Generalized Born/Volume Integral (GB/VI) methodology. After the homology modeling procedure has finished, the final model was inspected using MOE's Protein Geometry stereochemical quality evaluation tools including Ramachandran Maps.

### Immunohistochemistry Staining of Mouse HCC Tumor Slides

Huh 7 xenograft tumor slides were dewaxed with CitriSolv for 20 min, and 100%, 95%, 85%, 75% ethanol and dH₂O for 3 min for each. Peroxidase was blocked with 3% H₂O₂ for 10 min at RT. Slides were then washed thrice with PBS and target retrieval for 15 min under high temperature. Slides were then blocked with Avidin/Biotin blocking kit (Vector laboratories) following the manufacturer's instructions. Rabbit anti-mouse CD163 (Abcam, Ab6720) was diluted 1:200 and incubated with the slides at 4° C. overnight. Anti-rabbit secondary (Abcam) was diluted 1:200 and incubated with the slides at RT for 1 h. The signal was amplified with VECSTAIN ABC kit (Vector laboratories) and stained with DAB substrate kit (Vector laboratories) following the manufacturer's instructions. The stained slides were scanned using a Leica Aperio Slide Scanner and analyzed with QuPath as previously described.

ALDH1A1 (D9Q8E) IHC on human tissue. Five micron sections of formalin-fixed paraffin-embedded tissue were stained using and antibody against ALDH1A1 (D9Q8E, Cell Signaling 54135S, 1:400). Staining was done on a Leica Bond-III™ instrument using the Bond Polymer Refine Detection System (Leica Microsystems DS9800). Heat-induced epitope retrieval was done for 20 minutes with ER1 solution (Leica Microsystems AR9961). All the experiment was done at room temperature. Slides were washed three times between each step with bond wash buffer or water. Slides were scanned on a Hamamatsu NannoZoomerS360.

### Quantification and Statistical Analysis

Statistical significance was calculated between two groups by student's unpaired t test. One-way ANOVA with Tukey's HSD posttest was used to calculate statistical significance between multiple groups. Significance for survival was calculated by Kaplan-Meier with long-rank analysis. Analyses were performed using GraphPad Prism 8. Error bars represent SEM and p<0.05 was considered statistically significant (*p<0.05, **p<0.01, ***p<0.001).

### Example 1: HCC Expresses High Levels of RALDH1 and RA

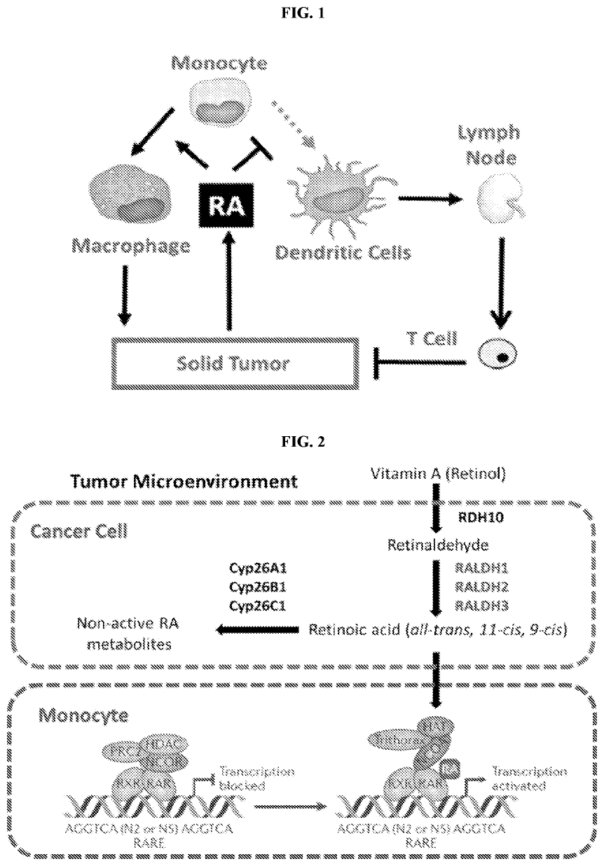

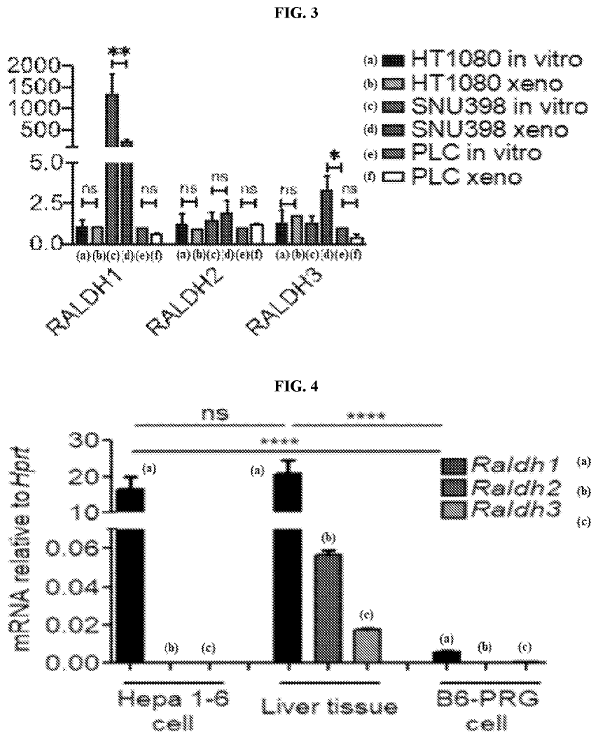

Recent work has identified an immune evasion pathway in solid tumors, wherein RA produced by tumor cells act on intratumoral monocytes to promote their differentiation into immunosuppressive tumor associated monocytes (TAMs) (FIG. 1). Therefore, blocking RA production by tumor cells and/or blocking RA signaling in monocytes may alleviate immunosuppression and engender antitumor immune responses. However, RA production and signaling display significant genetic redundancy. RALDH1, RALDH2, and RALDH3 each catalyzed the same rate-limiting step in RA production (FIG. 2). Since RA regulates many important developmental processes, non-specific inhibition of all RALDH isoforms is not a viable therapeutic approach. Hence, the gene expression profiles were in public databases were examined to identify tumors that may display selective overexpression of specific RALDH isoforms, resulting in the identification of HCC as selectively overexpressing RALDH1. The overexpression of RALDH1 was examined in human and murine cell lines (FIGS. 3-4). Notably, unlike liver tissue, HCC appears to rely almost exclusively on RALDH1 for generating RA (FIG. 4). Therefore, RALDH1 inhibition may reduce RA production in HCC without significantly affecting normal liver functions.

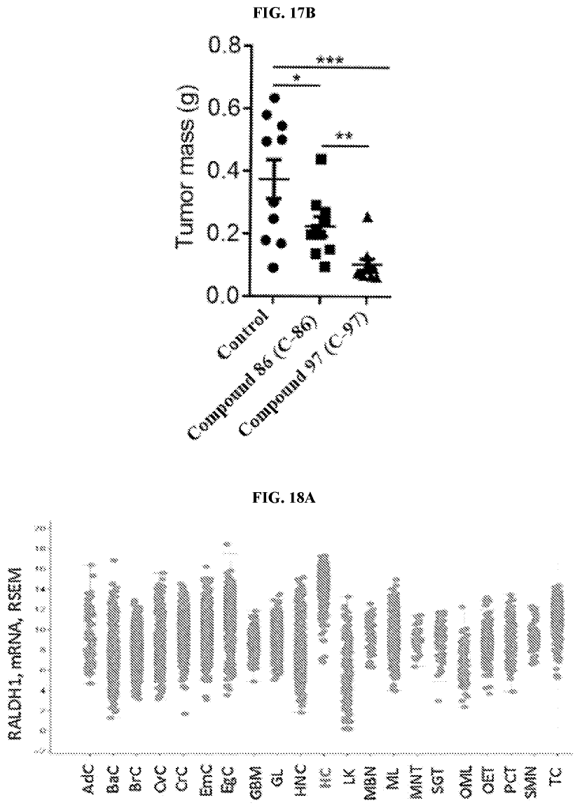

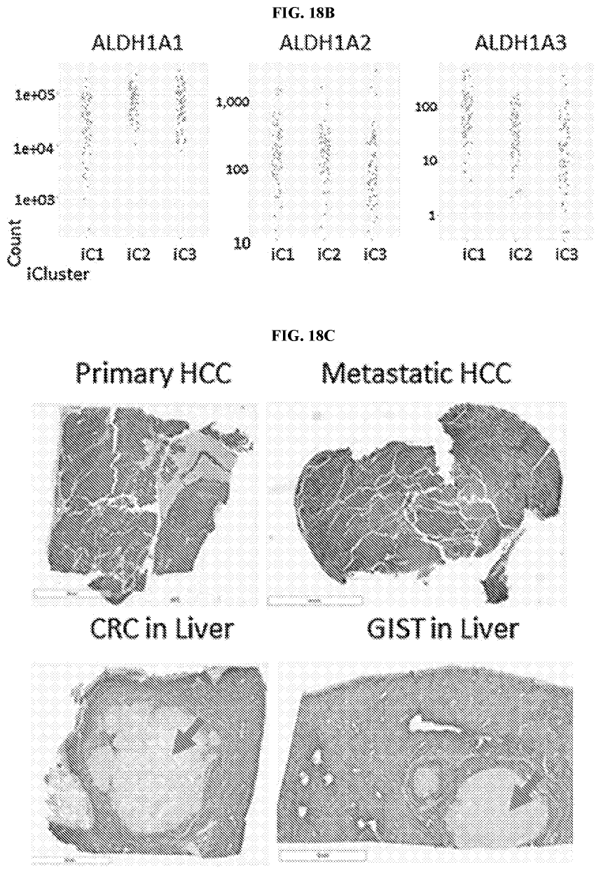

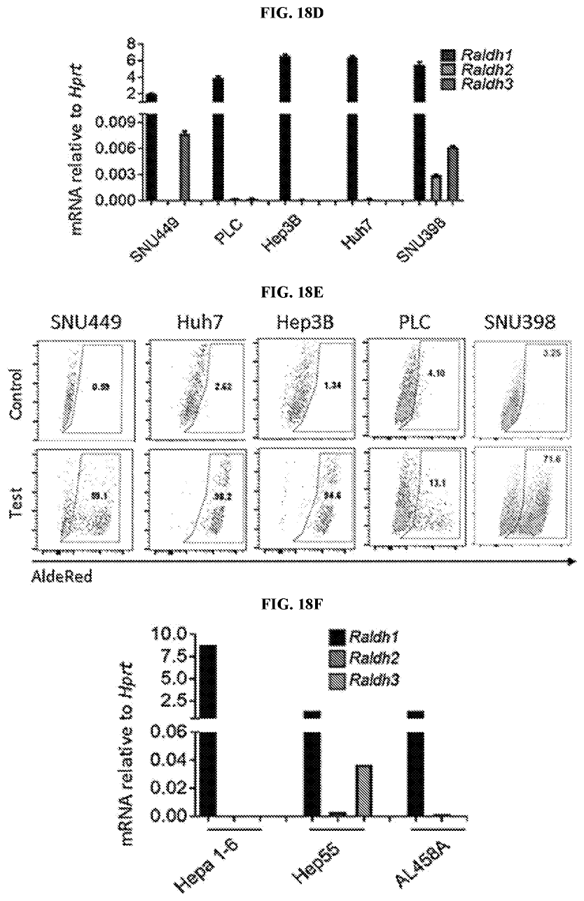

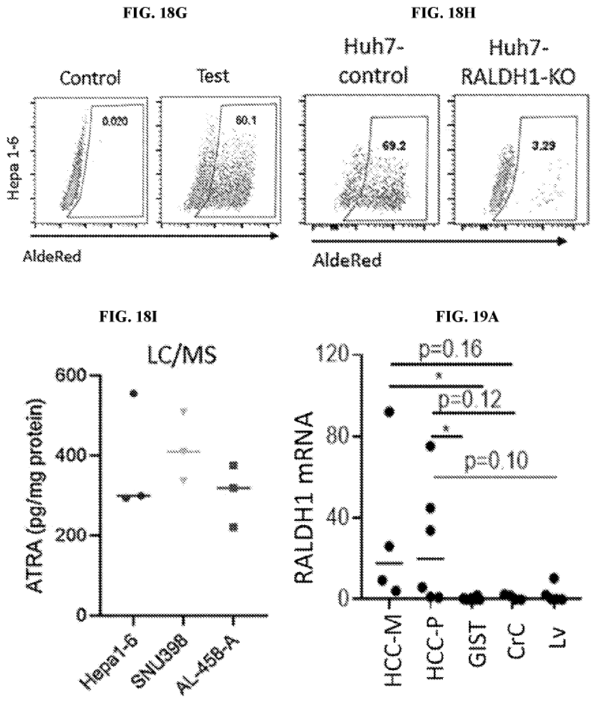

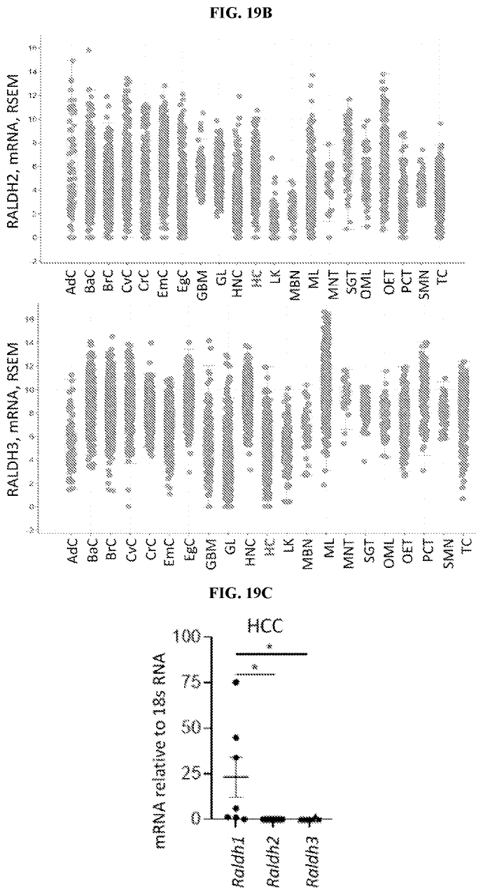

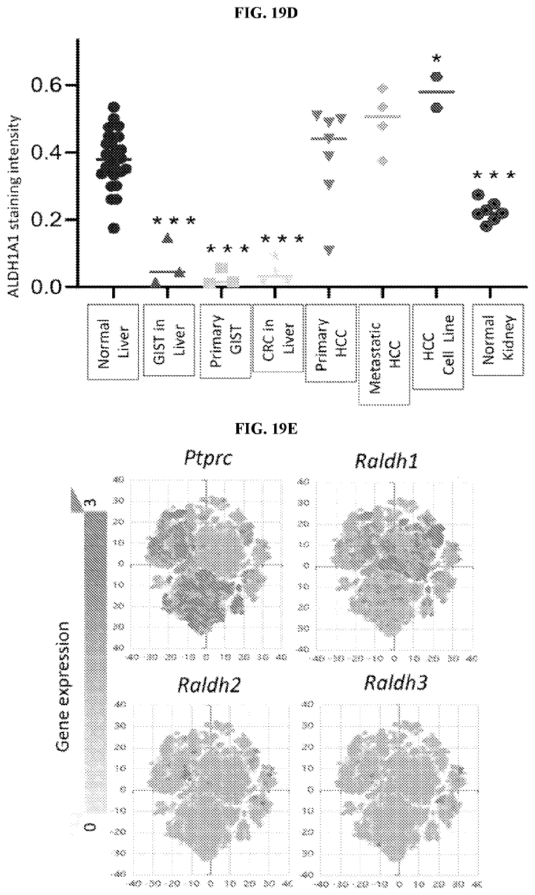

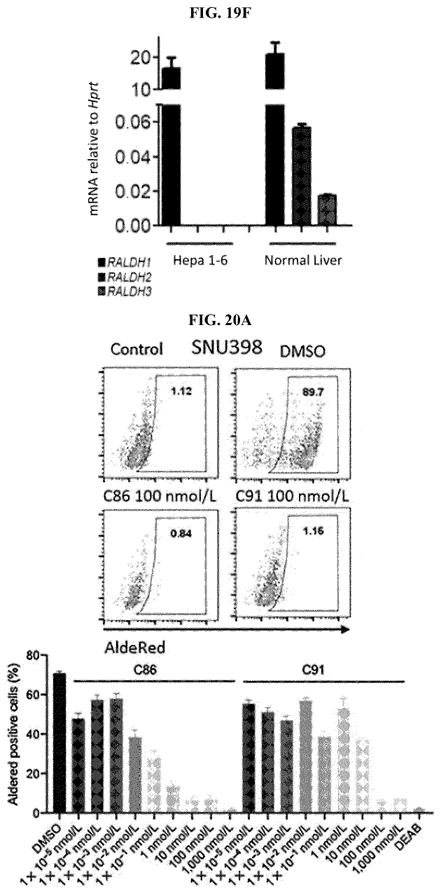

To explore whether other types of cancer display similar RA-dependent immune evasion, publicly available TCGA RNA-seq data from human tumors was analyzed for expression of RALDH isozymes, finding high RALDH1 transcripts in HCC (FIG. 18A). This was also confirmed through qRT-PCR in archived formalin fixed paraffin embedded (FFPE) specimens from human patients (FIG. 19A). In contrast, the other two RALDH isozymes were not highly expressed in HCC when compared to other tumors in the TCGA RNA-seq database (FIGS. 19B-19C). Next, it was examined whether high RALDH1 expression is associated with a specific subtype of HCC. A previous report described three distinct molecular subtypes of HCC based on DNA copy number, DNA methylation, mRNA expression, miRNA expression, and proteomics. Computational analyses of RNA-seq data downloaded from the aforementioned study showed high RALDH1, but not RALDH2 or RALDH3, in all subtypes, suggesting that RALDH1 overexpression is a hallmark of HCC (FIG. 18B). This was further confirmed by performing RALDH1 immunohistochemistry on primary and metastatic HCCs, as well as unrelated tumors, which revealed strong RALDH1 staining in HCCs (FIG. 18C and FIG. 19D).

The high level of expression of RALDH1 in HCC tumors may come from tumor cells, immune infiltrates, or other stromal components. To identify the primary source, a published and publicly available single cell RNA-seq (scRNA-seq) of human HCC was examined. Tumor cells and hepatocytes were the dominant producer of RALDH1 in this dataset and expressed low amounts of RALDH2 and 3 (FIG. 19E). Next, transcript levels of the three RALDH isozymes were measured in five distinct human HCC cell lines, finding high RALDH1 in all (FIG. 18D). To measure RALDH enzyme activity, an AldeRed assay was performed. Consistent with elevated RALDH1 transcripts, AldeRed positivity was detected in all human HCC cell lines tested (FIG. 18E). Likewise, murine HCCs also displayed high Raldh1 and AldeRed positivity (FIGS. 18F-18G). Of note, it was shown that the normal liver expresses all three Raldh isozymes while murine HCCs appear to loose/suppress Raldh2 and 3 (FIG. 18F). Thus, HCCs are likely dependent on RALDH1 for RA production. To confirm this, RALDH1 in human HCC cells was deleted using CRISPR/Cas9 and found dramatic reductions in AldeRed activity (FIG. 18H). Finally, high RA was confirmed in HCC cells through LC/MS based measurement of all-trans isomer of retinoic acid (ATRA), which is the dominant biologically active isomer of RA formed through RALDH1-catalyzed oxidation of Retinaldehyde (FIG. 18I). Taken together, results in this section demonstrate that HCCs produce high levels of RA via RALDH1.

### Example 2: C-86 and C-91 Inhibit RALDH1 Activity

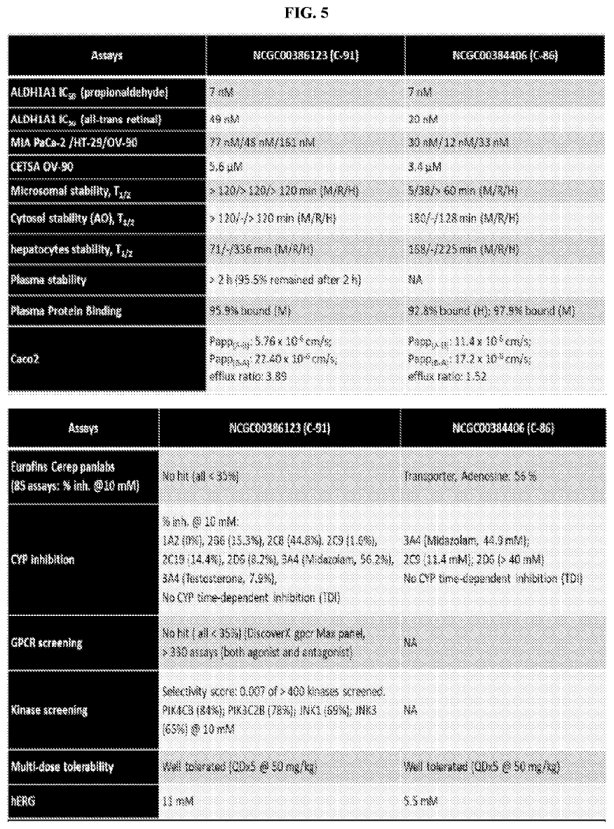

The human aldehyde dehydrogenase (ALDH) family comprises 19 isozymes, including the three isoforms (RALDH1, RALDH2, and RALDH3) that catalyze the conversion of retinaldehyde to RA. Given their role in RA production, there has been significant interest in the development of isozyme specific inhibitors of RALDH. Recently, two compounds have been identified as potent RALDH1 specific inhibitors, compound-86 (C-86) and compound-91 (C-91), which both have a particularly favorable profile (FIG. 5 and Table 6). Thus, C-86 and C-91 hold promise as drugs to reduce RA in HCC to promote anti-tumor immune responses.

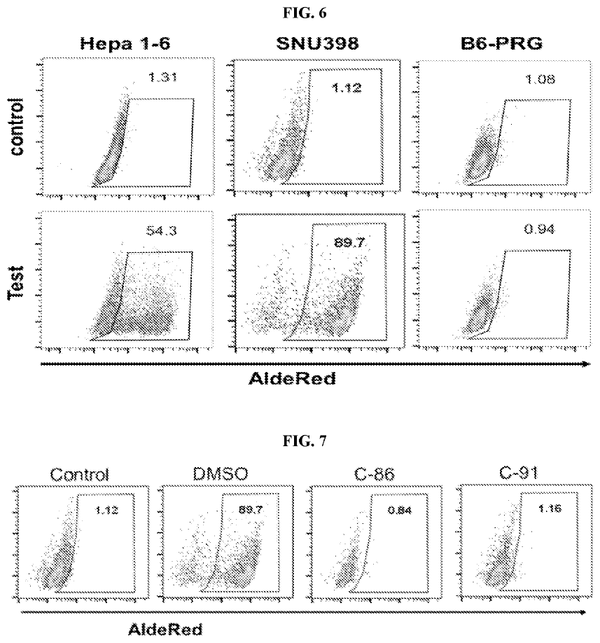

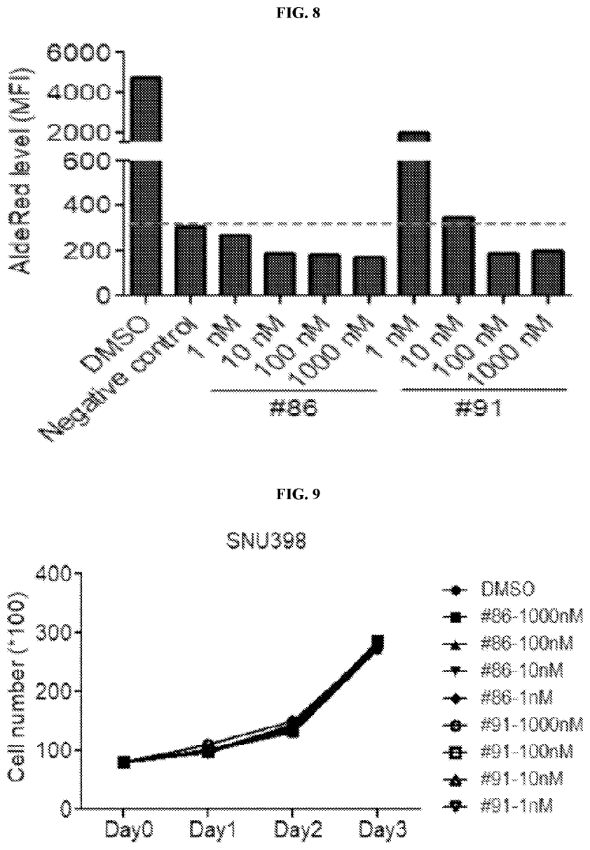

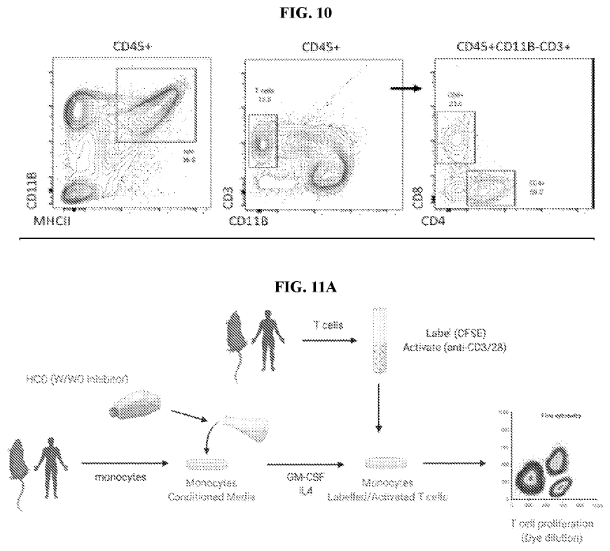

Consistent with high Raldh1 transcription (FIGS. 3-4), human and mouse HCC cell lines have demonstrated clear evidence of high RADLH activity as determined by Aldered assay (FIG. 6). Importantly, inhibition of RALDH1 via C-86 or C-91 led to significantly reduced RALDH activity, as determined by the Aldered assay, in HCC cells (FIGS. 7-8). Furthermore, inhibition with either compound did not inhibit HCC cell growth in vitro (FIG. 9). Taken together, these findings indicate that HCC produces high levels of RA via RALDH1 overexpression, which may be inhibited by C-86 and C-91.

Murine HCC tumors generated by Hepa 1-6 cells displayed significant infiltration by myeloid antigen-presenting cells and T cells (FIG. 10). The presence of significant numbers of T cells. Without wishing to be bound by theory, the data suggest that additional immunosuppressive pathways, such as RA-mediated immunosuppression, may be preventing these T cells from acting in HCC.

### Example 3: RALDH1 Inhibitors Fully Restore DC Differentiation from Monocytes in HCC Cell Lines

Many solid tumors produce high levels of RA which inhibit the formation of tumor-suppressive dendritic cells (DCs) through activation of retinoic acid receptors (RAR) and retinoid X receptors (RXR) receptors. As a result, the differentiation of tumor-permissive macrophages is promoted, thereby allowing tumor persistence and growth. When RA synthesis and/or signaling is inhibited, via inhibition of RALDH1 and/or RAR/RXR, respectively, monocytes differentiate into DCs that activate T cells, which in turn kill tumors such as hepatocellular carcinoma (HCC). Shifting the tumor microenvironment balance through inhibition of RA production is therefore desirable from a therapeutic perspective. Moreover, this approach may be combined with checkpoint inhibitors to enhance immune checkpoint blockage therapy.

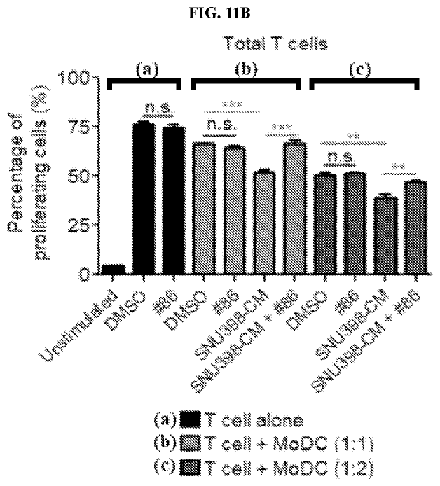

Utilizing the strategy described herein (FIG. 11A), restoration of activated T cell proliferation from monocytes exposed to HCC conditioned media was achieved using C-86. Addition of C-86 to MoDC and T cells exposed to SNU398-CM fully restored their proliferation rate, as compared to the vehicle (FIG. 111B). Thus, this represents the first use of RALDH1 inhibitors for cancer immunotherapy.

### Example 4: C-86 Inhibit In Vivo Growth of Hepatocellular Carcinoma Cell Line (Huh7)

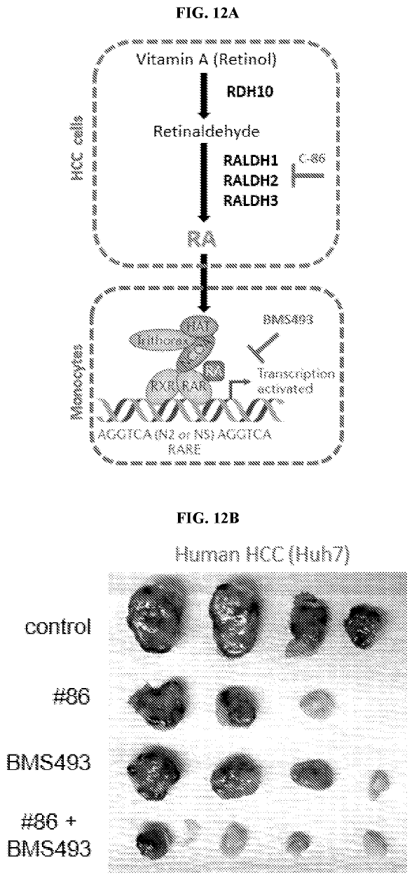

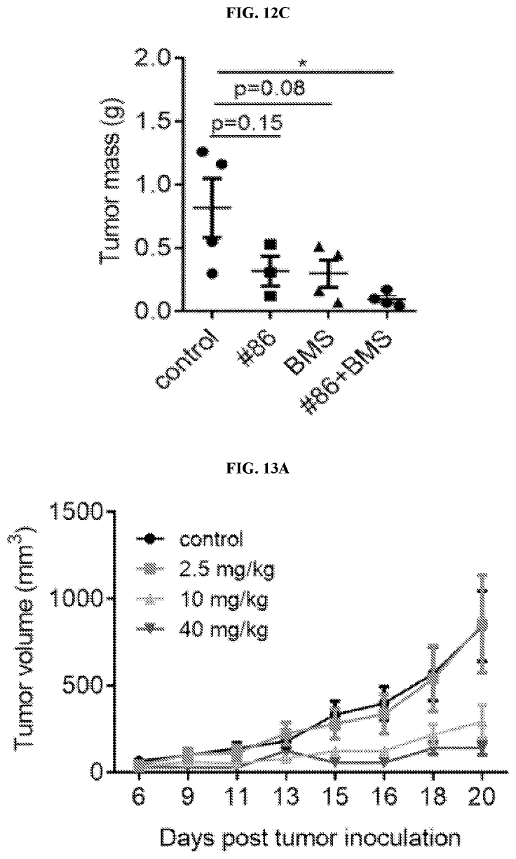

In one aspect, as described elsewhere herein the compounds of the present disclosure act to disrupt retinaldehyde (RA) synthesis by inhibition of RALDH1 (FIG. 12A). RA produced by tumor cells act on intratumoral monocytes to promote their differentiation into immunosuppressive tumor associated monocytes (TAMs). Thus, blocking RA production by tumor cells and/or blocking RA signaling in monocytes may alleviate immunosuppression and engender antitumor immune responses.

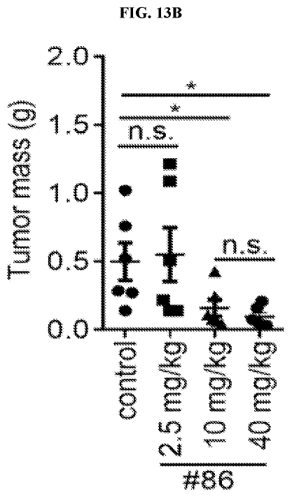

The tumor suppressive activity of compound C-86 was assessed against the Huh7 HCC cell line. Intraperitoneal administration of C-86, BMS 493, and C-86+BMS 493 each demonstrated a significant reduction in tumor mass and volume, as compared to a control, with the combination of C-86 and BMS 493 demonstrating a synergistic effect (FIGS. 12B-12C). Further, a dose-dependent effect on tumor volume and tumor mass was observed for C-86 (FIGS. 13A-13B).

### Example 5: In Vivo Effects of C-86 on Tumors May be Mediated by Macrophages

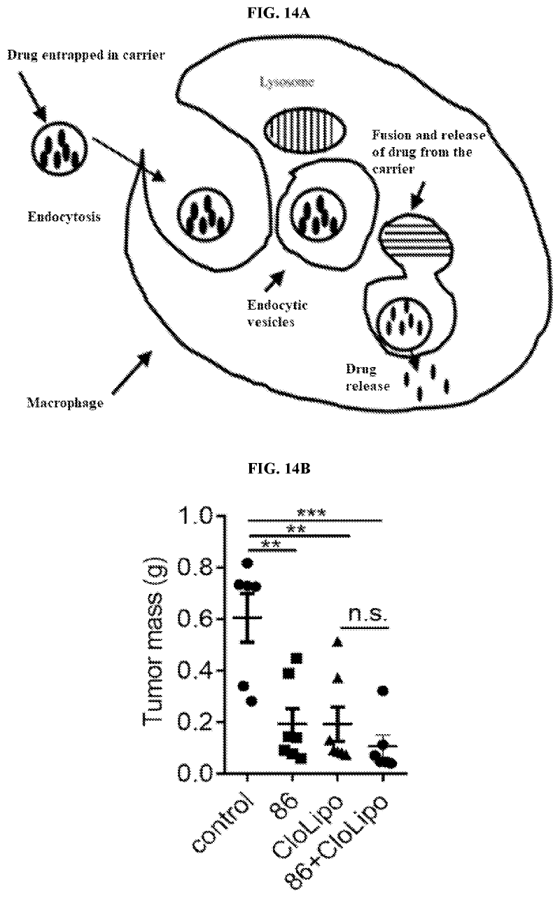

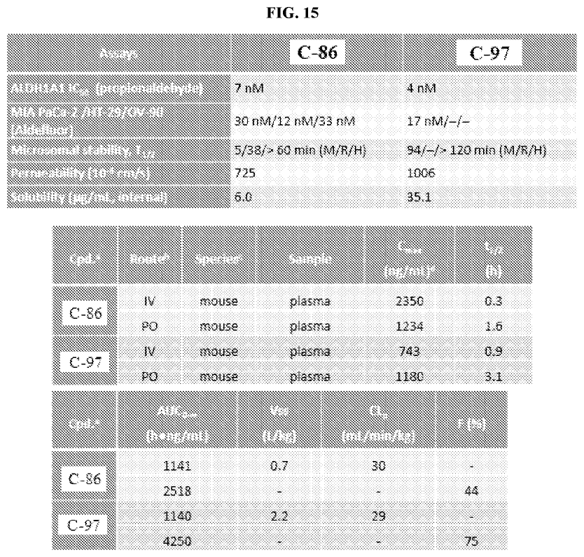

Without wishing to be bound by theory, it has been postulated that the in vivo effects of C-86 on tumors may be mediated by macrophages (FIG. 14A). Accordingly, tumor studies were performed using liposomal chlodronate (CloLipo) alone, or in combination with C-86. Liposomally encapsulated clodronate (CloLipo) has previously been found to be a potent antimacrophage agent. It selectively depletes animals of macrophages within 24 hours of administration by inducing apoptosis in these cells.

Intraperitoneal administration of C-86, CloLipo, and C-86+CloLipo each demonstrated a significant reduction in tumor mass, as compared to a control (FIG. 14B). Thus, macrophage depletion may contribute to the in vivo effects of C-86, and/or RALDH1 inhibitors broadly, on tumors.

### Example 6: C-97 Inhibits ALDH Activity in SNU98 Cells

The Aldefluor assay has been used to identify and isolate cells with high ALDH activities. This assay is based on the principle that ALDH can convert the ALDH-substrate, Bodipy-aminoacetaldehyde (BAAA) into Bodipy-aminoacetate (BAA) which is retained inside the cells. BAAA is uncharged and can diffuse freely into intact viable cells, however BAA cannot cross the membrane due to its net negative charge, which makes remain in the cells and the assay buffer prevents efflux of the BAA from the cells. Therefore, the amount of BAA fluorescence in cells is proportional to ALDH activity and can be measured using a flow cytometer. A specific inhibitor of ALDH, diethylaminobenzaldehyde (DEAB), is used for background fluorescence control.

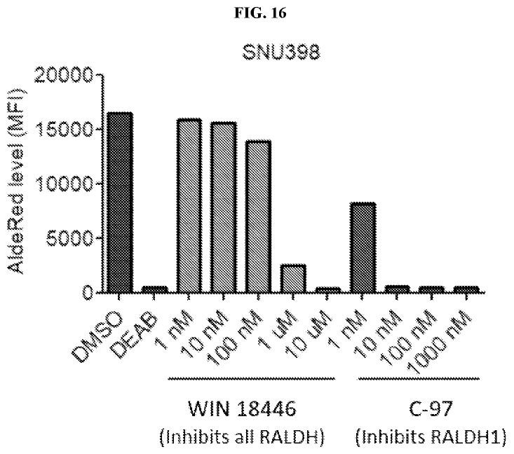

Administration of C-97 at 1 nM resulted in reduced ALDH1 activity compared to the DMSO control (i.e., partial inhibition of ALDH activity at 1 nM C-97), with essentially complete inhibition of ALDH at concentrations at or above 10 nM (FIG. 16). Thus, C-97 potently inhibits ALDH activity.

### Example 7: C-97 Inhibits In Vivo Growth of Hepatocellular Carcinoma Cell Line (Huh7)

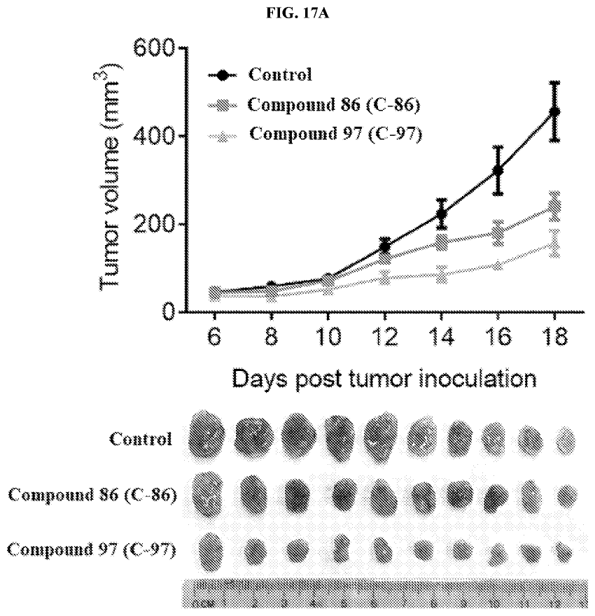

The relative tumor suppressive activity of compound C-97, as compared to C-86, was assessed against the Huh7 HCC cell line. Intraperitoneal administration of C-86 and C-97 each demonstrated a significant reduction in tumor mass and volume, as compared to a control (FIGS. 17A-17B). Further, compound C-97 demonstrated enhanced tumor suppressive activity as compared to C-86.

### Example 8: Activity of Selected Compounds as RALDH1 Inhibitors

Compounds ALDH inhibitory effects of C-9125, C-9163, and C-9175 were evaluated in a number of cell lines, including MiaPaCa2 and OV90 cell lines, utilizing the AldeFlour assay described elsewhere herein (Table 4).

TABLE 4

| | | MiaPaCa | OV90 |
|---|---|---|---|
| | hALDHIA1 | cells | cells |
| Cmpd | IC₅₀ (μM) | IC₅₀ (μM) | IC₅₀ (μM) |
| C-9125 | 0.007 | 0.13 | N/A |
| C-9163 | 0.004 | 0.093 | N/A |
| C-9175 | 0.006 | 0.012 | N/A |

### Example 9: RALDH1 Inhibitors Abrogate RA Production in HCC Cells

RA can drive autocrine or paracrine signaling by binding RAR/RXR transcription factor heterodimers to regulate gene expression. Thus, reducing RA production by inhibiting RALDH enzymes and/or blocking RA signaling through RAR/RXR has the potential to curtail the RA-mediated tumor immune evasion previously described. However, RA is an important morphogen and signaling molecule, which precludes global RA blockade as a therapeutic strategy. RAR and RXR have several isoforms that generate a diverse repertoire of RAR/RXR heterodimers. While isoform specific inhibitors of RARs and RXRs have been developed, they are used as tool compounds due to toxicity and lack of approved clinical indications.

In contrast, RALDH isozyme-specific inhibitors as a strategy for RA blockade have not been adequately explored. Two best-in-class RALDH1 inhibitors (Raldh1-INH), Compounds 86 (C86) and 91 (C91), also known as NCT-505 and NCT-506, respectively, showed PK and pharmacodynamic profiles favorable to potential clinical applications. Thus, it was examined whether C86 and/or C91 could inhibit RA production in HCC cells.

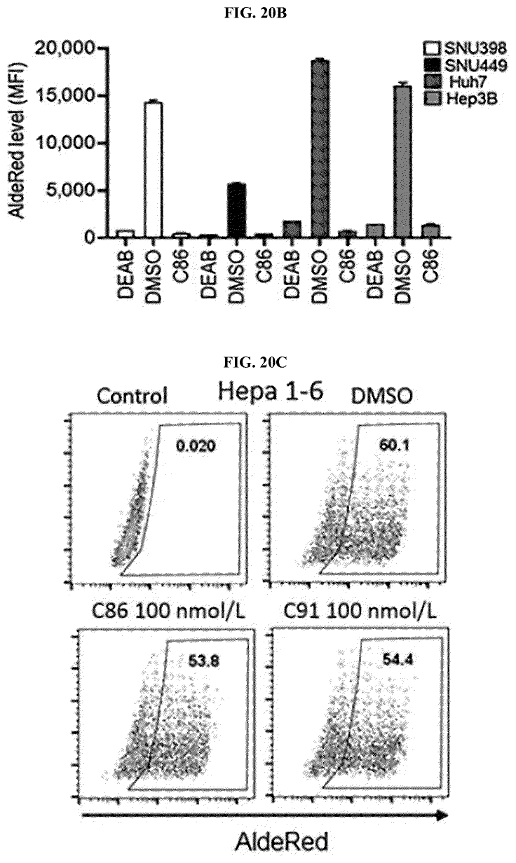

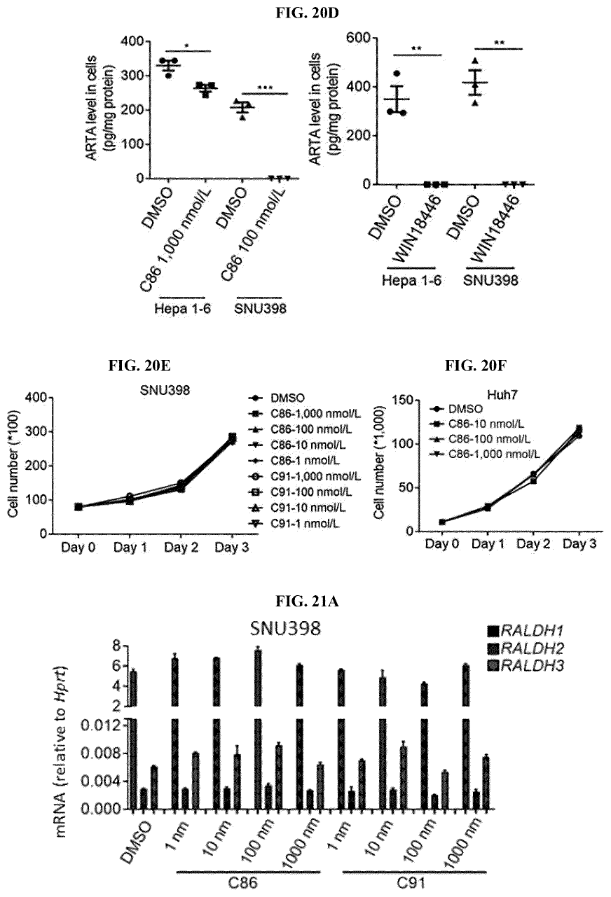

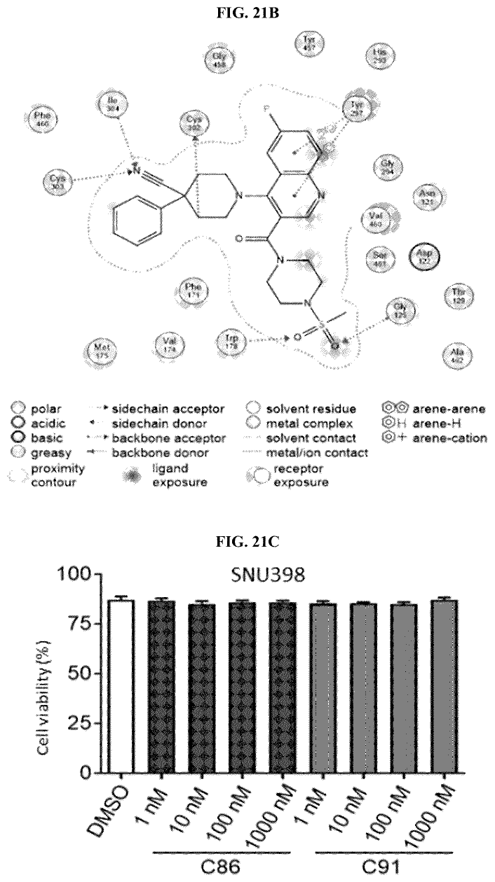

Both inhibitors reduced AldeRed fluorescence in human HCCs, although C86 displayed higher potency (FIGS. 20A-20B and FIG. 5). Thus, C86 was primarily used in subsequent experiments. C86 or C91 did not lead to compensatory increases in transcription of any other RALDH isozymes, which is consistent with the AldeRed data and demonstrates the efficacy of these inhibitors in suppressing RA production in HCC (FIG. 21A).

In contrast to human HCC, both inhibitors failed to reduce AldeRed fluorescence in murine HCC cells (FIG. 20C). LC/MS-based RA measurements confirmed the lack of RA suppression by C86 in murine HCC (FIG. 20D). Thus, C86 and C91 activity show species specificity. Homology modeling based on partial crystal structure of these Raldh1-INH suggest that differences in key drug-interacting amino acids between mouse and human may underlie this observation (FIG. 21B). Finally, it was confirmed that the reduced RA and AldeRed activity in human HCC cell lines with C86 or C91 were not due to increased cell death or reduced cell viability (FIGS. 20E-20F and FIG. 21C). Thus, results in this section demonstrate the efficacy of Raldh1-INH in abrogating RA production in human HCC cell lines.

### Example 10: HCC-Derived RA Regulates Monocyte Differentiation

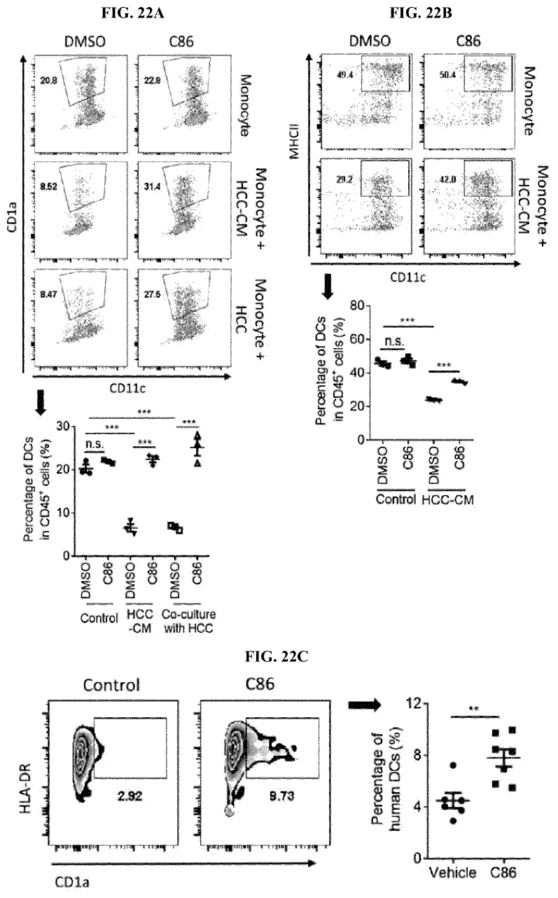

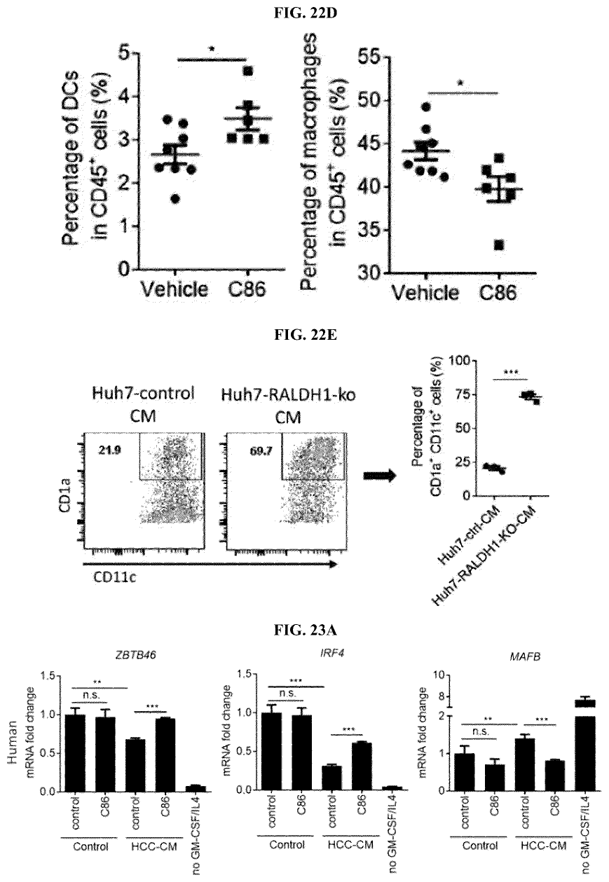

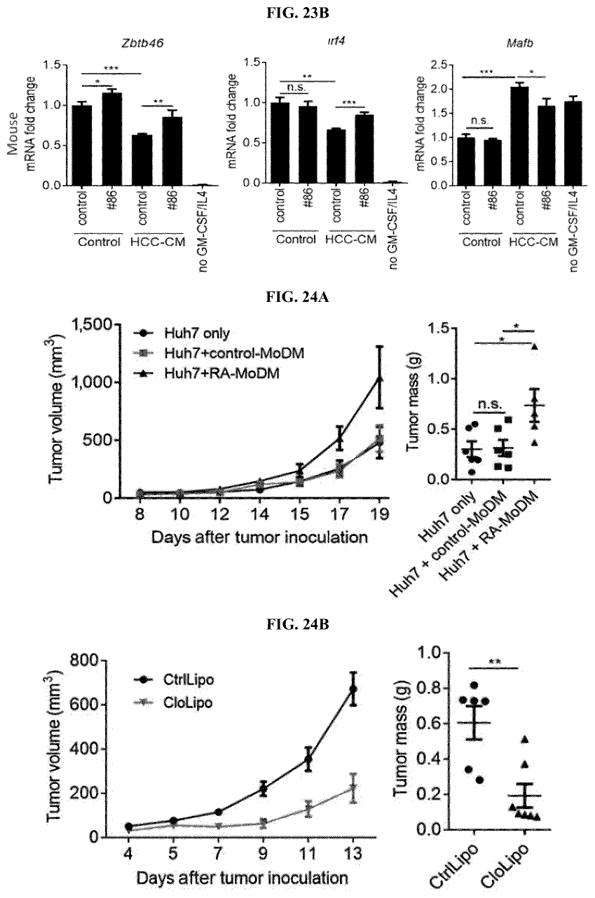

To examine whether HCCs regulate monocyte differentiation, human monocytes were co-cultured with either human HCC cell lines or cell culture supernatant (conditioned media, CM) from these cell lines. Flow cytometry analysis showed suppression of DC differentiation in the presence of HCC cells or CM (FIG. 22A). qRT-PCR analyses confirmed this, showing suppression of DC-associated genes and increased expression of macrophage-associated genes with CM (FIG. 22A). These findings were reproduced in murine monocytes cultured with HCC cells or CM (FIG. 22B and FIG. 23B). The effects were reversed when HCC cells were pre-treated with the Raldh1-INH C86 (FIGS. 22A-22B and FIGS. S23A-23B). Thus, HCC-derived RA regulates monocyte differentiation in vitro, which can be ‘rescued’ by blocking RA production through Raldh1-INH.

Next, this was tested in vivo by transplanting immunodeficient (NU/J) mice with the human HCC cell line Huh7 and, once the tumors were established, performing intratumoral injection with primary human monocytes. Mice were treated with vehicle (control) or C86. In this setting, C86 selectively inhibited RALDH1 in the transplanted human cells (HCC and human monocytes) due to the species-specificity of this inhibitor described elsewhere herein. Five days after monocyte transplantation the tumors were analyzed by flow cytometry where human and murine leukocytes were distinguished using standard species-specific anti-CD45. Raldh1-INH treatment increased DC differentiation from transplanted human monocytes (FIG. 22C). Without wishing to be bound by any theory, this may reflect the effect of reducing RA production in HCC cells and is consistent with results from the co-culture experiments described elsewhere herein. Although a direct effect of the inhibitor on the transplanted human monocytes could also explain this observation, this is unlikely given that the host (murine) immune cells in the TME, which are insensitive to C86, also showed increased frequency of DCs and reduced frequency of macrophages (FIG. 22D). Furthermore, C86 treatment of monocytes in vitro did not alter their potential to differentiate into DCs (FIG. 22A). Finally, RA was confirmed as the key mediator of the aforementioned effects of HCCs on monocyte differentiation by using RALDH1-KO HCC cells, whereby CM from the knockout cells failed to suppress DC differentiation (FIG. 22E). Taken together, data presented in this section show that Raldh1-INH suppresses HCC RA production and its attendant impact on monocyte differentiation.

### Example 11: RA Induces Tumor-Promoting Attributes in Monocyte-Derived Macrophages

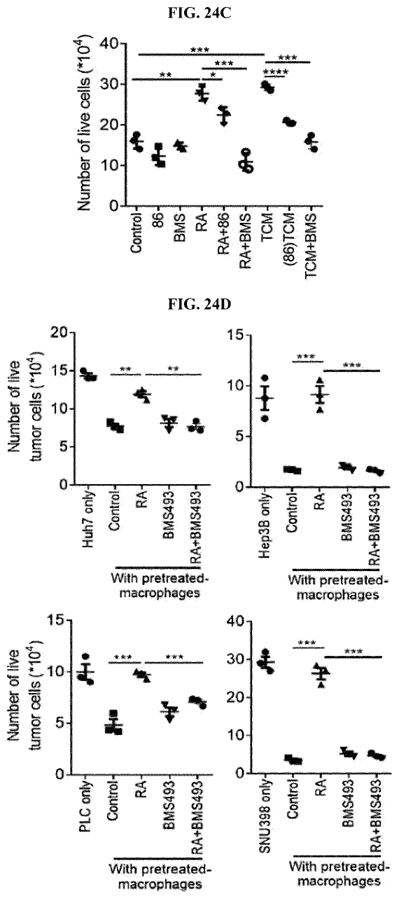

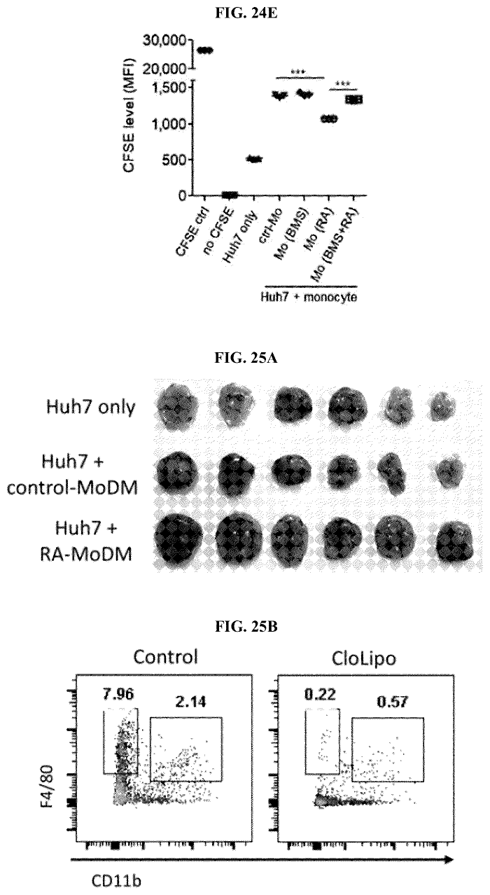

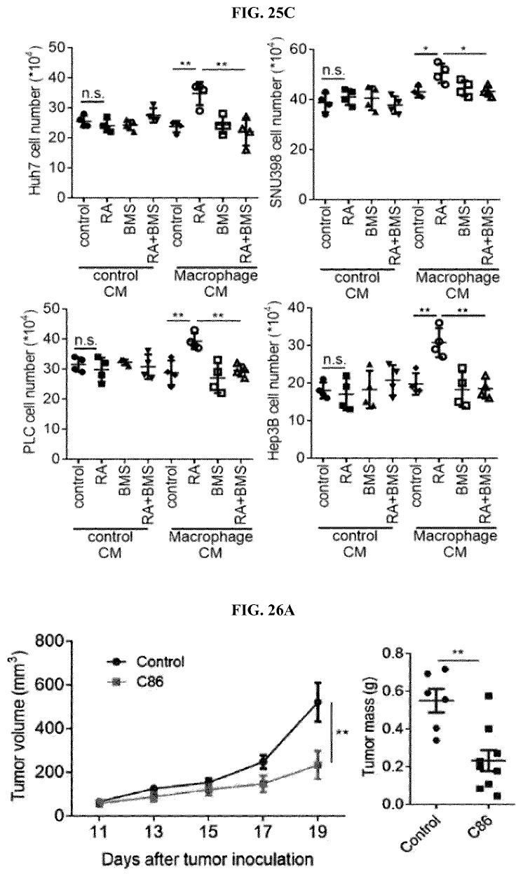

Monocytes can differentiate into macrophages or DCs and the adaptive immune consequences of RA-mediated suppression of DC differentiation from monocytes in tumor immunity are described elsewhere herein. However, whether and how tumors are affected by RA-induced macrophages remained unclear. As described elsewhere herein, HCC-derived RA can increase macrophage frequency in the TME (FIG. 22D). To examine whether RA also alters macrophage function, a macrophage-tumor co-transplantation approach was utilized. Primary human monocytes were differentiated into macrophages in the presence or absence of RA, mixed 50:50 with the human HCC cell line (Huh7), and transplanted into immunodeficient NU/J mice. HCC cells transplanted without macrophages served as an additional control. RA-treated macrophages accelerated tumor growth compared to HCC cells transplanted alone or with control macrophages (FIG. 24A and FIG. 25A). Therefore, the overall impact of macrophages in HCC TME was next assessed by depleting TAMs through i.p. liposomal clodronate (CloLipo), which is a common method to deplete macrophages. TAM reductions with CloLipo were confirmed, and reduced tumor growth was observed under these conditions (FIG. 24B and FIG. 25B). Thus, HCC-associated TAMs support tumor growth, a property that could be induced by high RA in the HCC TME.

To further explore this, a reductionist approach of co-culturing RA-induced macrophages with HCC cells was taken. First, it was observed that RA exposure increased macrophage numbers, which is consistent with in vivo observations in the TME and suggests that RA may increase macrophage proliferation and/or survival (FIG. 24C). RA pre-treated macrophages significantly increased tumor cell numbers compared to non-treated control macrophages; this effect was reversed when the macrophages were exposed to RAR signaling inhibitor BMS493 (FIG. 24D). Consistent with increased tumor cell numbers, CFSE labeling suggested increased tumor cell proliferation in the presence of RA-treated macrophages when compared to untreated control macrophages (FIG. 24E). These effects were recapitulated when HCC cells were grown with CM from RA-treated or control macrophages, suggesting that RA exposure may lead to production of a soluble ‘mitogenic’ factor by macrophages (FIG. 25C). Of note, tumor cells showed reduced proliferation when cultured with control (untreated) monocytes/macrophages, likely due to competition for nutrients (FIG. 24D). Pre-treatment with RA abolished this suppressive effect, indicating the tumor-supportive effect of RA exposure. The experiment with CM (FIG. 25C) clarified this further as the absence of monocytes/macrophages eliminated this competition for nutrients and ‘unmasked’ the mitogenic effect of the soluble factor.

To summarize, the results described herein show that HCC-derived RA induces TAMs to produce factors that support HCC growth.

### Example 12: RALDH1 Inhibitors Suppress HCC Growth

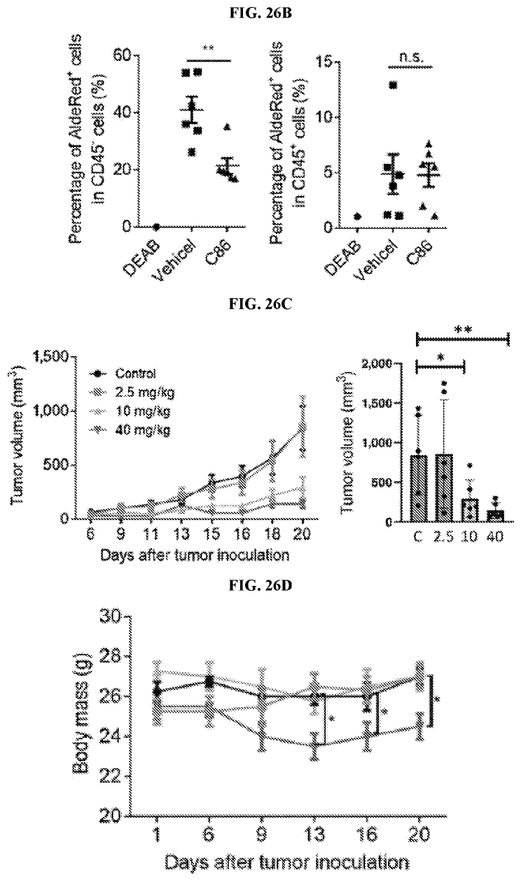

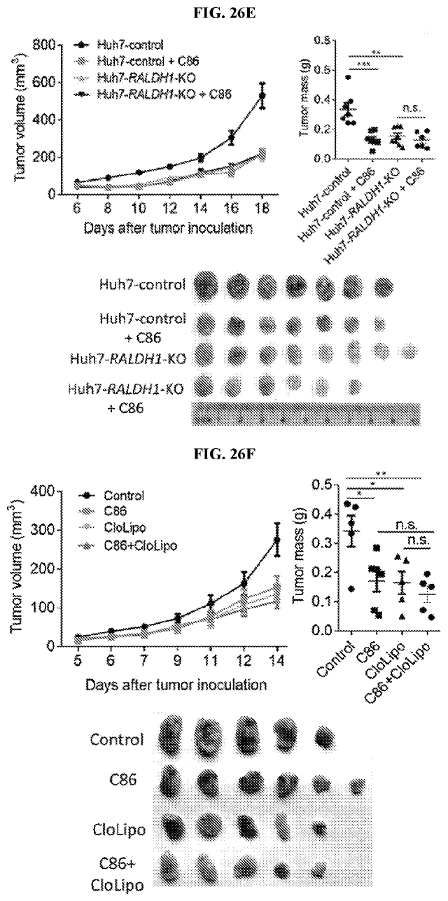

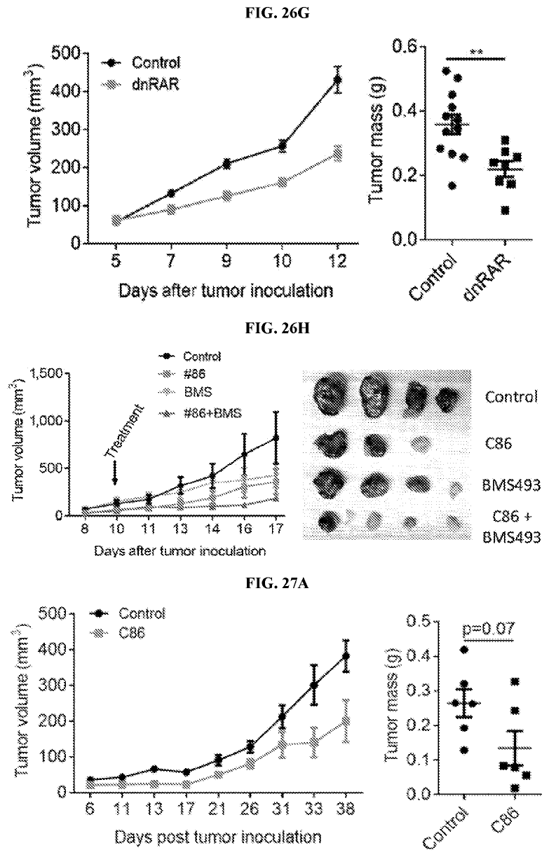

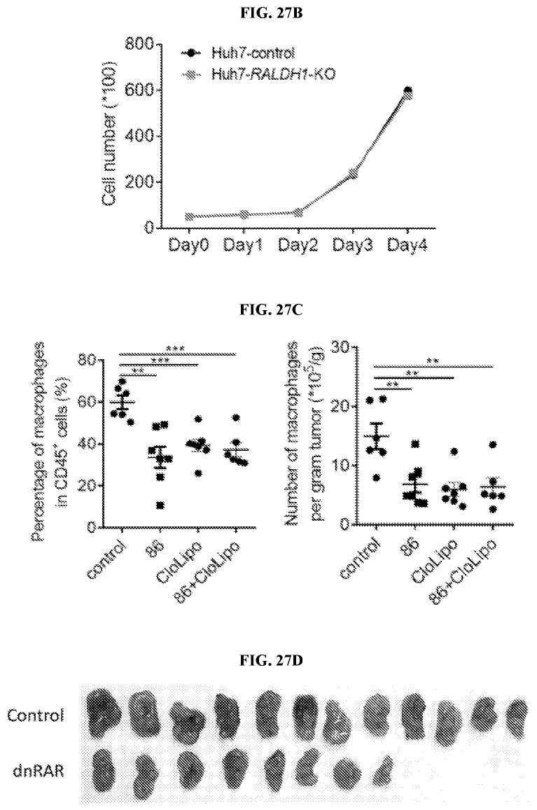

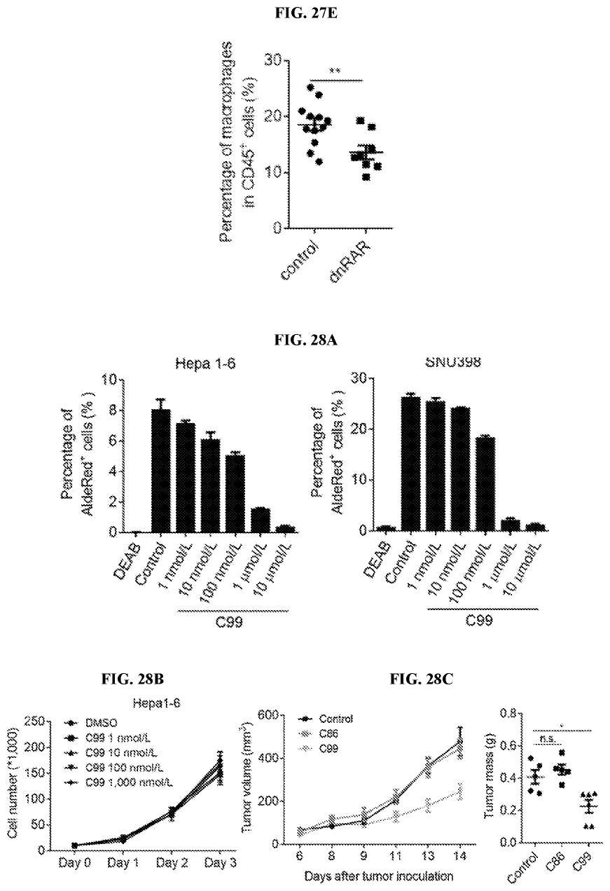

The data described elsewhere herein show that Raldh1-INH can alter monocyte differentiation and macrophage functions in the TME by blocking RA production in HCC cells. To examine the therapeutic implications, huh1 and huh7 human HCC cell lines were transplanted into immunodeficient mice and the mice were treated with C86, with significant tumor inhibition observed (FIG. 26A and FIG. 27A). C86 treatment reduced RADLH activity, and hence RA production, in tumor cells but not infiltrating leukocytes (FIG. 26B). C86 displayed a dose-response, with tumor suppressive effects at >10 mg/kg once daily dosing and weight loss observed only at higher doses of 40 mg/kg (FIG. 26C-26D). Correspondingly, deletion of RALDH1 with CRISPR/Cas9 (RALDH1-KO) in HCC cells led to slower tumor growth in vivo, but not in vitro (FIG. 26E and FIG. 27B). RALDH1-KO HCC tumors did not respond to C86 therapy, demonstrating that the tumor-suppressive effects of Raldh1-INH were primarily driven by on-target effects on RALDH1 (FIG. 26E). Next, the role of TAMs in mediating the therapeutic effects of C86 was examined by depleting TAMs through CloLipo treatment. TAM depletion suppressed HCC growth and C86 treatment did not further suppress tumors in the absence of TAMs (FIG. 26F and FIG. 27C). Thus, the therapeutic effects of reducing HCC-derived RA require the presence of TAMs.

Next, it was tested whether HCC-derived RA acts directly on TAMs to promote tumor growth observed above. Towards this goal, mice were obtained that conditionally express a dominant-negative isoform of RAR from the Rosa26 locus (dnRAR^{flox} mouse). Expression of dnRAR leads to inhibition of RAR-mediated effects of RA. dnRAR^{flox} was crossed to Lysozyme 2-Cre mice (LysM^{Cre}), which express Cre recombinase in myeloid cells, including macrophages. Human HCC cells (Huh7) were transplanted after T-cell depletion into control and Lysm^{Cre}: dnRAR^{flox} mice, revealing significant slowing of tumor growth and reduced TAMs with myeloid-specific dnRAR expression (FIG. 26G and FIGS. 27D-27E). This suggests that tumor-derived RA induces tumor-promoting TAMs, which is consistent with data provided elsewhere herein. Nonetheless, dnRAR-mediated suppression of RA signaling is partial as it only inhibits RAR-mediated pathways and not the other RAR isoforms or RXRs, and very high levels of RA can still overcome RAR-inhibition by dnRAR.

To further examine the impact of blocking RAR signaling, especially in the context of therapy, Huh7-bearing mice were treated with the pan-RAR blocker BMS493 alone or in combination with Raldh1-INH. While monotherapy with BMS493 or Raldh1-INH slowed tumor growth, the combination showed the greatest effect (FIG. 26H). Thus, blocking RA production through Raldh1-INH and/or RA signaling through RAR inhibitors can suppress HCC growth.

### Example 13: Reducing Tumor-Derived RA is the Primary Mechanism of Tumor Suppression by Raldh1-INH

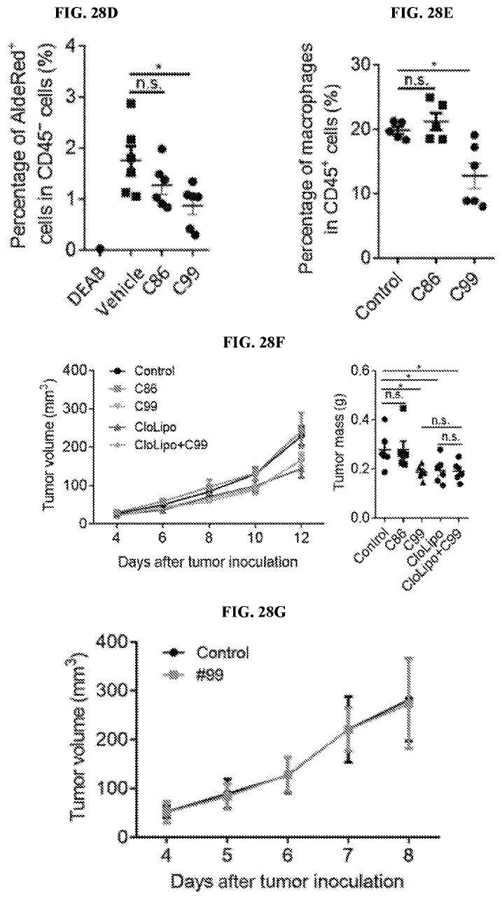

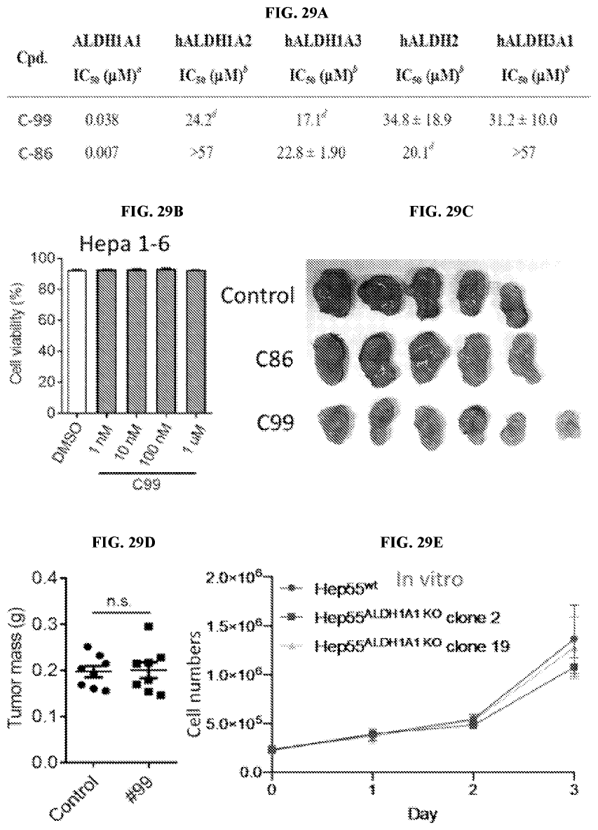

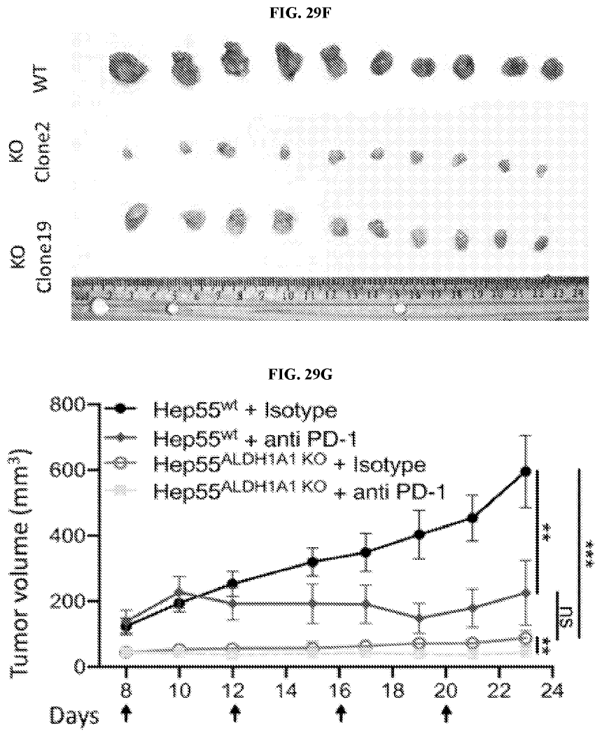

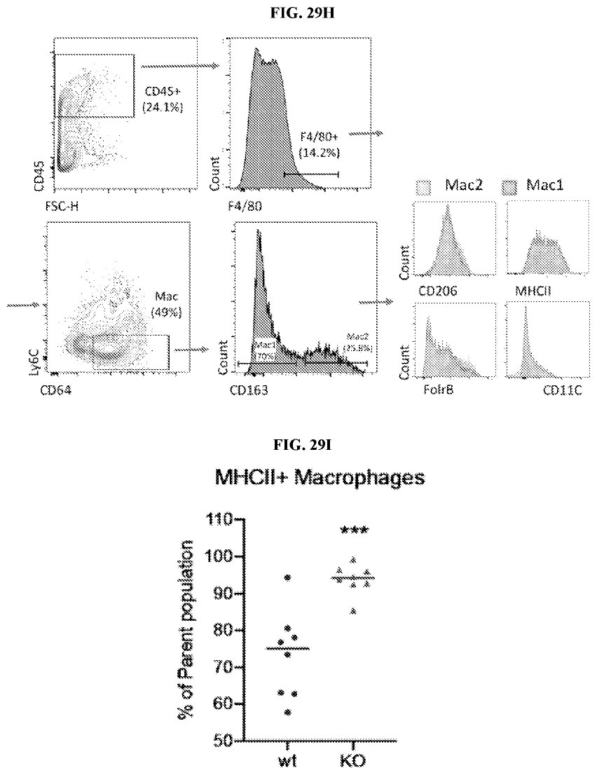

As described elsewhere herein, C86 and C91 show species specificity and do not inhibit murine RALDH1 (FIGS. 20C-20D). Thus, the aforementioned therapeutic effects on xenotransplantation-based tumor models represent a scenario where the drug can only work on transplanted tumor cells but not host cells and there are no T cells to mount antitumor immune responses. To overcome these limitations, compound-99 (C99), a potential inhibitor of murine RALDH1, was selected from an earlier chemical series for further evaluation. In vitro, C99 suppressed RALDH1 activity in both murine and human HCC cell line (FIG. 28A), albeit at much higher (micromolar, FIG. 28A) concentrations compared to C86 (nanomolar range, FIG. 20A). Correspondingly, the IC₅₀ of C99 was found to be significantly greater than that of C86 (FIG. 29A). Despite lower potency, C99 permitted the aforementioned limitations of cross-species xenotransplantation models to be circumvented, providing an opportunity to further probe the biological responses to Raldh1-INH.

C99 did not reduce proliferation or viability of the murine HCC cell line Hepa 1-6 in vitro, but it significantly suppressed tumor growth in vivo (FIGS. 28B-28C and FIGS. 29B-29C). The human-specific C86 did not suppress murine Hepa 1-6 growth in vivo (FIG. 28C). C99 treatment, but not C86 treatment, reduced AldeRed activity in tumor cells and the frequency of TAMs within the TME (FIGS. 28D-28E). Furthermore, TAM depletion with CloLipo suppressed Hepa 1-6 tumor growth and rendered the tumors insensitive to C99 treatment (FIG. 28F). These findings mirror the effects of C86 on human HCC described elsewhere herein and suggest that the tumor suppressive activity of RALDH1 inhibitors is dependent on their ability to block RA production in tumor cells. To further confirm this, C99 was tested on the FS murine model of fibrosarcoma that expresses high levels of both Raldh1 and 3 and is not solely dependent on RALDH1 for RA production; C99 did not suppress fibrosarcoma growth (FIG. 28G and FIG. 29D). Taken together, data in this section demonstrate the efficacy of Raldh1-INH in suppressing HCC growth through inhibition of RA production.

### Example 14: Raldh1-INH for HCC Immunotherapy

As described elsewhere herein, C86 monotherapy showed therapeutic effects even in the absence of T cells in xenotransplanted tumor models. C99 also suppressed tumor growth in syngeneic tumor models with intact adaptive immunity, but with two major limitations: (1) C99 is less potent than C86 and C91; and (2) the murine Hepa 1-6 tumor cells tend to generate significant T-cell responses after subcutaneous transplantation in C57BL6/J mice, which occasionally leads to spontaneous delayed tumor rejection. These limitations make it difficult to examine the true therapeutic potential of C99 with Hepa 1-6. Nonetheless, it was considered important to examine the impact of full RALDH1 inhibition in the presence of T cells and test combinations with ICB. Towards this goal, Hep55 was identified as a murine HCC cell line that shows less spontaneous T-cell responses and no rejection. As shown in FIG. 18F, Hep55 cells also have high Raldh1 expression.

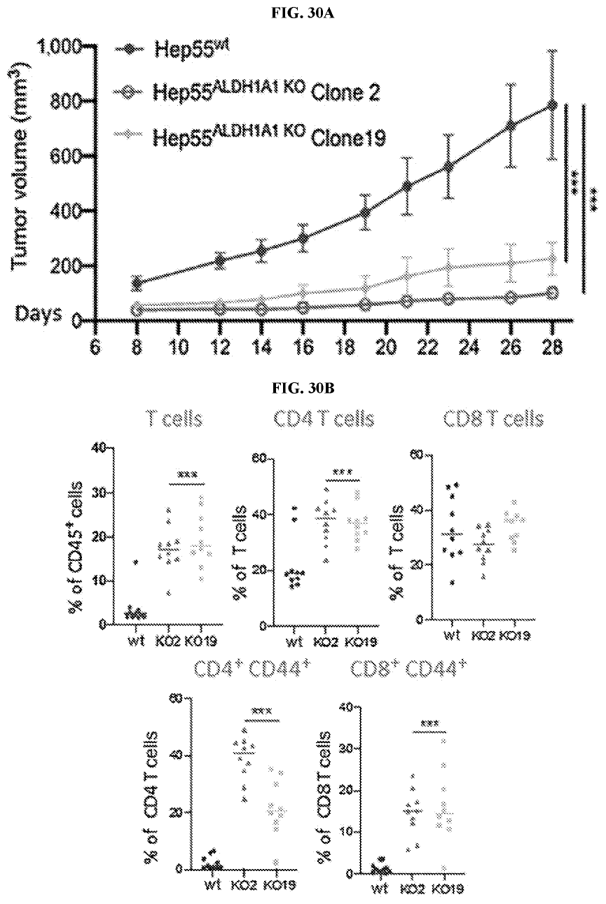

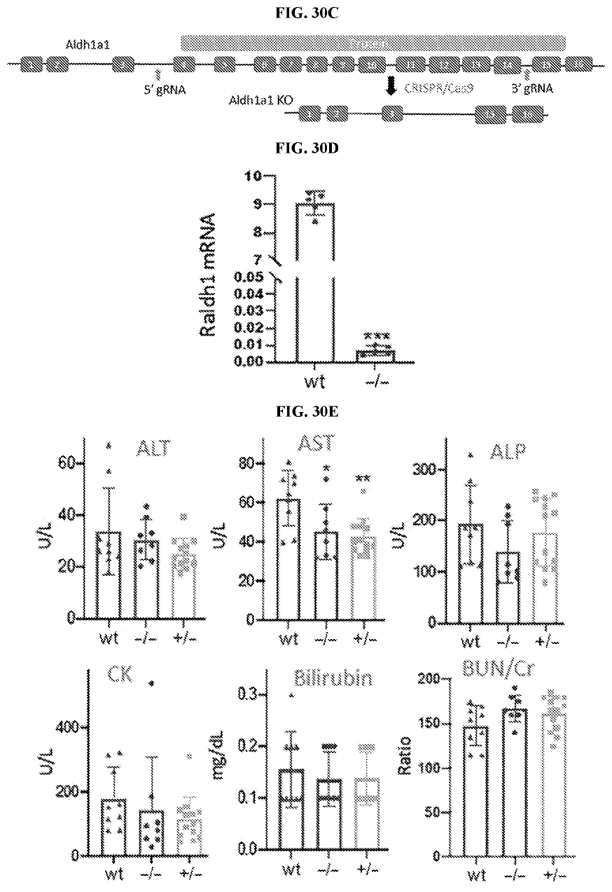

To overcome the limited efficacy of C99, a genetic approach was used to create a RALDH1 deletion in Hep55 using CRISPR/Cas9. Loss of Raldh1 gene was compatible with normal growth of Hep55 cells in vitro but led to profound tumor suppression when the cells were transplanted in vivo (FIG. 30A and FIGS. 29E-29F). Tumor suppression was accompanied by significantly enhanced infiltration of RALDH1-KO tumors with activated T cells (FIG. 30B). Anti-PD1 treatment led to an even greater suppression of growth by RALDH1-KO Hep55 tumors (FIG. 29G). Hep55 tumors showed infiltration with both pro- and anti-inflammatory macrophages and the frequency of pro-inflammatory macrophages increased significantly with the loss of RALDH1 activity in tumor cells (FIGS. 29H-29I). These findings are consistent with both macrophages and T cells driving the therapeutic effects of RALDH1 inhibition. In this context, it is worth noting that growth of RALDH1-KO Hep55 tumors was suppressed to a greater extent than C86 treatment suppressed the growth of xenotransplanted human HCC. Without wishing to be bound by any theory, this may be due to a greater extent of RALDH1 inhibition with a genetic knockout, however, a more likely explanation is the presence of T cells in the syngeneic Hep55 model.

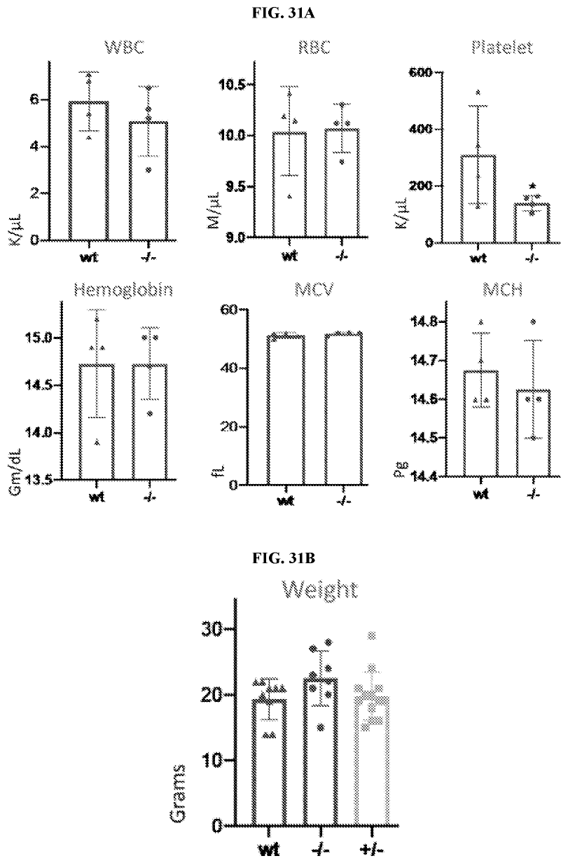

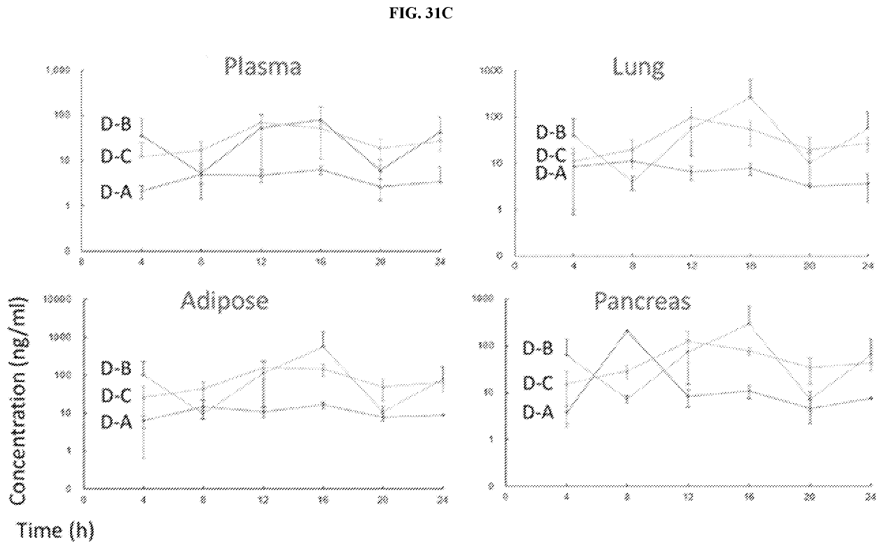

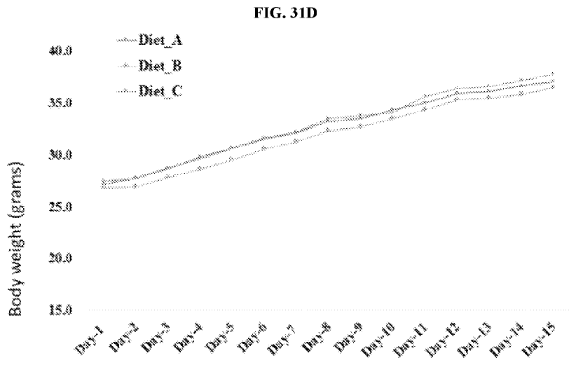

An important issue for the use of Raldh1-INH is on-target toxicity, especially given the expression of RALDH1 in normal liver. Although no signs of toxicity have been observed with C86 and C91, a major caveat is the species specificity of these compounds. To examine the potential for on-target toxicity of RADH1 inhibition, a genetic deletion of RALDH1 in mice was prepared (FIGS. 30C-30D). RALDH1-KO mice did not show any overt toxicity and developed normally compared to their WT siblings. Basic toxicological analyses of serum and complete blood count also did not reveal any abnormalities in RALDH1-KO and there were no significant weight differences between genotypes (FIG. 30E FIGS. 31A-31B). A standard panel of in vitro assays were performed for off target assays (Eurofins Cereps Panlabs 85), that showed no significant concerns for off-target effects at the expected therapeutic concentrations (Table 5). Thus, RALDH1-INH tested here are unlikely to cause serious toxicity.

TABLE 5
Safetyscreen assay for C86

| Assay Name | Species | No. | Conc | % Inh. |
|---|---|---|---|---|
| ATPase, Na+/K+, Heart, Pig | pig | 2 | 10 μM | 2 |
| Cholinesterase, Acetyl, ACES | hum | 2 | 10 μM | 3 |
| Cyclooxygenase COX-1 | hum | 2 | 10 μM | 6 |
| Cyclooxygenase COX-2 | hum | 2 | 10 μM | 0 |
| Monoamine Oxidase MAO-A | hum | 2 | 10 μM | 2 |
| Monoamine Oxidase MAO-B | hum | 2 | 10 μM | 4 |
| Peptidase, Angiotensin Converting Enzyme | rabbit | 2 | 10 μM | −6 |
| Peptidase, CTSG (Cathepsin G) | hum | 2 | 10 μM | 0 |
| Phosphodiesterase PDE3 | hum | 2 | 10 μM | −23 |
| Phosphodiesterase PDE4 | hum | 2 | 10 μM | −7 |
| Protein Serine/Threonine Kinase, | rat | 2 | 10 μM | 3 |
| PKC, Non-Selective | | | | |
| Protein Tyrosine Kinase, Insulin Receptor | hum | 2 | 10 μM | 0 |
| Protein Tyrosine Kinase, LCK | hum | 2 | 10 μM | 3 |
| Adenosine A1 | hum | 2 | 10 μM | 4 |
| Adenosine A2A | hum | 2 | 10 μM | 14 |
| Adrenergic α1A | rat | 2 | 10 μM | 6 |
| Adrenergic α1B | rat | 2 | 10 μM | 6 |
| Adrenergic α1D | hum | 2 | 10 μM | 6 |
| Adrenergic α2A | hum | 2 | 10 μM | 0 |
| Adrenergic α2B | hum | 2 | 10 μM | 9 |
| Adrenergic β1 | hum | 2 | 10 μM | 3 |
| Adrenergic β2 | hum | 2 | 10 μM | 11 |
| Androgen (Testosterone) | hum | 2 | 10 μM | 16 |
| Angiotensin AT1 | hum | 2 | 10 μM | 19 |
| Bradykinin B2 | hum | 2 | 10 μM | 2 |
| Calcium Channel L-Type, Benzothiazepine | rat | 2 | 10 μM | −3 |
| Calcium Channel L-Type, Dihydropyridine | rat | 2 | 10 μM | 23 |
| Calcium Channel L-Type, Phenylalkylamine | rat | 2 | 10 μM | 21 |
| Calcium Channel N-Type | rat | 2 | 10 μM | 6 |
| Cannabinoid CB1 | hum | 2 | 10 μM | 14 |
| Cannabinoid CB2 | hum | 2 | 10 μM | 21 |
| Chemokine CCR1 | hum | 2 | 10 μM | 18 |
| Chemokine CXCR2 (IL-8RB) | hum | 2 | 10 μM | 8 |
| GABAA, Ro-15-1788, Hippocampus | rat | 2 | 10 μM | −7 |
| GABABIA | hum | 2 | 10 μM | 7 |
| Glucocorticoid | hum | 2 | 10 μM | 9 |
| Glutamate, AMPA | rat | 2 | 10 μM | 0 |
| Glutamate, Kainate | rat | 2 | 10 μM | 11 |
| Glutamate, Metabotropic, mGlu5 | hum | 2 | 10 μM | 16 |
| Glutamate, NMDA, Agonism | rat | 2 | 10 μM | 2 |
| Glutamate, NMDA, Glycine | rat | 2 | 10 μM | 20 |
| Glutamate, NMDA, Phencyclidine | rat | 2 | 10 μM | 5 |
| Glutamate, NMDA, Polyamine | rat | 2 | 10 μM | 1 |
| Glycine, Strychnine-Sensitive | rat | 2 | 10 μM | 23 |
| Histamine H1 | hum | 2 | 10 μM | 13 |
| Histamine H2 | hum | 2 | 10 μM | −3 |
| Leukotriene, Cysteinyl CysLT1 | hum | 2 | 10 μM | 5 |
| Melanocortin MC1 | hum | 2 | 10 μM | 8 |
| Melanocortin MC4 | hum | 2 | 10 μM | 13 |
| Muscarinic M1 | hum | 2 | 10 μM | 3 |
| Muscarinic M2 | hum | 2 | 10 μM | 4 |
| Muscarinic M3 | hum | 2 | 10 μM | 5 |
| Muscarinic M4 | hum | 2 | 10 μM | 13 |
| Neuropeptide Y Y1 | hum | 2 | 10 μM | 11 |
| Nicotinic Acetylcholine | hum | 2 | 10 μM | 5 |
| Nicotinic Acetylcholine α1, Bungarotoxin | hum | 2 | 10 μM | 2 |
| Opiate δ1 (OP1, DOP) | hum | 2 | 10 μM | 2 |
| Opiate κ(OP2, KOP) | hum | 2 | 10 μM | 41 |
| Opiate μ(OP3, MOP) | hum | 2 | 10 μM | 19 |
| Platelet Activating Factor (PAF) | hum | 2 | 10 μM | 32 |
| Potassium Channel [KATP] | ham | 2 | 10 μM | 28 |
| Potassium Channel hERG | hum | 2 | 10 μM | 29 |
| PPARγ | hum | 2 | 10 μM | 12 |
| Progesterone PR-B | hum | 2 | 10 μM | 1 |
| Serotonin (5-Hydroxytryptamine) 5-HT1A | hum | 2 | 10 μM | 5 |
| Serotonin (5-Hydroxytryptamine) 5-HT1B | hum | 2 | 10 μM | −21 |
| Serotonin (5-Hydroxytryptamine) 5-HT2A | hum | 2 | 10 μM | 36 |
| Serotonin (5-Hydroxytryptamine) 5-HT2B | hum | 2 | 10 μM | −5 |
| Serotonin (5-Hydroxytryptamine) 5-HT2C | hum | 2 | 10 μM | 22 |
| Serotonin (5-Hydroxytryptamine) 5-HT3 | hum | 2 | 10 μM | 2 |
| Sodium Channel, Site 2 | rat | 2 | 10 μM | 21 |
| Tachykinin NK1 | hum | 2 | 10 μM | 37 |
| Transporter, Adenosine | gp | 2 | 10 μM | 56 |
| Transporter, Dopamine (DAT) | hum | 2 | 10 μM | 8 |
| Transporter, GABA | rat | 2 | 10 μM | 4 |
| Transporter, Norepinephrine (NET) | hum | 2 | 10 μM | 35 |
| Transporter, Serotonin | hum | 2 | 10 μM | 17 |
| (5-Hydroxytryptamine) (SERT) | | | | |
| Vasopressin V1A | hum | 2 | 10 μM | −13 |

Given the good efficacy and toxicity profile of RALDH1-INH, PK and pharmacodynamic (PD) properties of three exemplary RALDH1-inhibitors (i.e., C86, C91, and C99) were examined. Here, it was found that the half-life of C99 was also significantly lesser than the other two exemplary Raldh1-INH (Table 6). While a better IC50 of C86 compared to C91 (FIG. 5) was the rationale for using C86 in all experiments, it was found that the half-life of C86 was lower than that of C91 when the compounds were given through oral (PO) or intravenous (IV) routes (Table 6). Nonetheless, i.p. delivery was used in all in vivo experiments above, which is associated with superior pharmacokinetic profile compared to PO or IV route (Tables 6-7).

TABLE 6
Exemplary pharmacokinetic and pharmacodynamic
data for certain compounds^{a}

| | Cₘₐₓ | t_{1/2} | AUC_{0-∞} | Vₛₛ | CLₚ | F |
|---|---|---|---|---|---|---|
| Compound | (ng/mL) | (h) | (h × ng/mL) | (L/kg) | (ml/min/kg) | (%) |
| C-91^{b} | 1997 | 1.3 | 1830 | 1.4 | 18 | — |
| C-91^{c} | 1530 | 2.2 | 6980 | — | — | 76 |
| C-86^{b} | 2350 | 0.3 | 1141 | 0.7 | 30 | — |
| C-86^{c} | 1234 | 1.6 | 2518 | — | — | 44 |
| C-99^{b} | 117 | 0.1 | 37 | 2.7 | 887 | — |
| C-99^{c} | 16 | 0.5 | 11 | — | — | 6.3 |

^{a}studies utilized mouse plasma;
^{b}intravenous administration;
^{c}oral administration.

TABLE 7
Exemplary pharmacokinetic and pharmacodynamic
data for C-86 in mice

| | | | | | | AUC |
|---|---|---|---|---|---|---|
| | | | | AUC_{0-∞} | | ratio |
| | | Cₘₐₓ | t_{1/2} | (h × | AUC | (30/10 |
| Route | Sample | (ng/mL) | (h) | ng/mL) | ratio | mg/kg) |
| IP, 10 mg/kg | plasma | 2930 | 1.4 | 3720 | | |
| IP, 10 mg/kg | brain | 362 | 3.3 | 753 | B/P = 0.20 | |
| IP, 10 mg/kg | liver | 27600 | 3 | 32900 | L/P = 8.8 | |
| IP, 10 mg/kg | pancreas | 34400 | 2.3 | 35000 | Pa/P = 9.4 | |
| IP, 10 mg/kg | adipose^{a} | 45200 | 3 | 121000 | A/P = 33 | |
| IP, 30 mg/kg | plasma | 11600 | 1.6 | 28000 | | 7.5 |
| IP, 30 mg/kg | brain | 1150 | 4.3 | 4520 | B/P = 0.16 | 6 |
| IP, 30 mg/kg | liver | 70500 | 3.6 | 174000 | L/P = 6.2 | 5.3 |
| IP, 30 mg/kg | pancreas | 149000 | 3.8 | 276000 | Pa/P = 9.8 | 7.9 |
| IP, 30 mg/kg | adipose^{a} | 140000 | 3.2 | 832000 | A/P = 30 | 6.9 |

^{a}mesenteric

C86 also showed good bio-distribution in various tissues when delivered i.p. (Table 7). Meanwhile, chow was formulated with C86 and examined PK/PD of the inhibitor over a 15-day period (FIG. 31C). This approach showed good stability of C86 in chow and consistent drug exposure over time without affecting body weight (FIGS. 31C-31D). Thus, Raldh1-INHs show good efficacy and PK/PD profile.

In summary, data described herein establish RALDH1 as a bonafide therapeutic target for HCC immunotherapy. Its efficacy as monotherapy is demonstrated. Additionally, given its unique mechanism of action, there are opportunities for combination with other treatment approaches. As a proof of concept, additive effects with ICB were demonstrated. Finally, the present disclosure stablishes RALDH inhibition as a viable therapeutic approach in other cancers.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### ENUMERATED EMBODIMENTS

The following exemplary embodiments are provided, the numbering of which is not to be construed as designating levels of importance:

Embodiment 1 provides a method of treating, preventing, and/or ameliorating a solid tumor in a subject in need thereof, the method comprising administering to the subject a pharmaceutically effective amount of:
(a) at least one immunostimulator; and
(b) a retinaldehyde dehydrogenase 1 (RALDH1) inhibitor, wherein the RALDH1 inhibitor is selected from the group consisting of:
(i) a compound of formula (I), or a salt, solvate, prodrug, stereoisomer, tautomer, or isotopologue thereof:

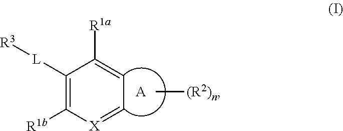

wherein:
R^{1a} is selected from the group consisting of optionally substituted C₂-C₈ heterocyclyl, optionally substituted phenyl, and optionally substituted C₅-C₈ cycloalkenyl,
   wherein each optional substituent in R^{1a} is independently selected from the group consisting of C₁-C₆ alkyl, C₃-C₆ cycloalkyl, C₁-C₆ alkoxy, optionally substituted phenyl, optionally substituted C₂-C₈ heterocyclyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b},
      wherein each optional substituent is optionally substituted with at least one substituent selected from the group consisting of C₁-C₃ alkyl, C₁-C₃ alkoxy, halogen, CN, and NO₂, and
   wherein two vicinal or geminal optional substituents in R^{1a} may combine with the atoms to which they are bound to form a C₂-C₈ heterocyclyl or C₃-C₈ cycloalkyl;
R^{1b} and R^{1c}, if present, are each independently selected from the group consisting of H, C₁-C₆ alkyl, C₁-C₆ alkoxy, halogen, OH, N(R^{a})(R^{b}), NO₂, and CN;
each occurrence of R² is independently selected from the group consisting of C₁-C₆ alkyl, C₁-C₆ alkoxy, C₁-C₃ haloalkoxy, C₁-C₆ hydroxyalkyl, halogen, NO₂, and CN;
R³ is selected from the group consisting of optionally substituted C₂-C₈ heterocyclyl, optionally substituted phenyl, N(R^{a})(optionally substituted C₃-C₈ cycloalkyl), and N(R^{a})(optionally substituted C₂-C₈ heterocyclyl),
   wherein each optional substituent in R³ is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b};
A is selected from the group consisting of optionally substituted C₆-C₁₀ aryl and optionally substituted C₂-C₈ heterocyclyl,
   wherein each optional substituent in A is independently selected from the group consisting of C₁-C₆ alkyl, C₁-C₆ alkoxy, C₁-C₆ hydroxyalkyl, halogen, OH, N(R)(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b}
L is selected from the group consisting of —CH₂—, —C(═O)—, and —S(═O)₂—;
X is selected from the group consisting of N and CR^{1c};
n is an integer selected from the group consisting of 0, 1, 2, 3, and 4; and
each occurrence of R^{a}, R^{b}, and R^{c} is independently selected from the group consisting of H, C₁-C₆ alkyl, C₂-C₆ alkenyl, C₂-C₆ alkynyl, C₃-C₈ cycloalkyl, and C₂-C₈ heterocyclyl; and
(ii) a compound selected from the group consisting of:
   1-benzylindoline-2,3-dione;
   ethyl 2-((4-oxo-3-(3-(pyrrolidin-1-yl)propyl)-3,4-dihydrobenzo[4,5]thieno[3,2-d]pyrimidin-2-yl)thio)acetate;
   2-((2-(sec-butyl)-3-oxo-2,3-dihydroimidazo[1,2-c]quinazolin-5-yl)thio)-N-(o-tolyl)butanamide; and
   8-((4-(cyclopropanecarbonyl)piperazin-1-yl)methyl)-7-isopentyl-1,3-dimethyl-3,7-dihydro-1H-purine-2,6-dione;
   or a salt, solvate, prodrug, stereoisomer, tautomer, or isotopologue thereof.

Embodiment 2 provides the method of Embodiment 1, wherein the RALDH1 inhibitor is a compound of formula (I).

Embodiment 3 provides the method of Embodiment 2, wherein the compound of formula (I) is selected from the group consisting of:

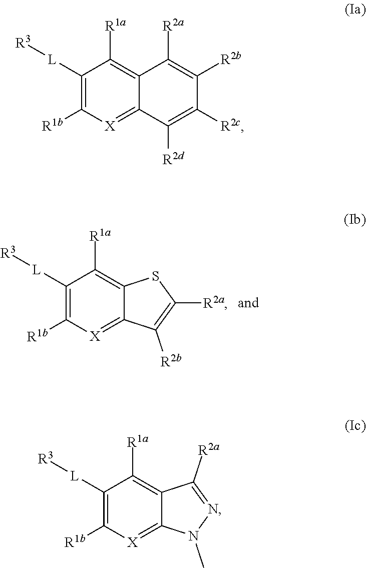

wherein:
R^{2a}, R^{2b}, R^{2c}, and R^{2d}, if present, are each independently selected from the group consisting of H, C₁-C₆ alkyl, C₁-C₆ alkoxy, and halogen.

Embodiment 4 provides the method of Embodiment 3, wherein R^{2a}, R^{2b}, R^{2c}, and R^{2d}, if present, are each independently selected from the group consisting of H, Me, OMe, F, and Cl.

Embodiment 5 provides the method of any one of Embodiments 2-4, wherein X is N.

Embodiment 6 provides the method of any one of Embodiments 2-5, wherein R^{1a} is selected from the group consisting of:

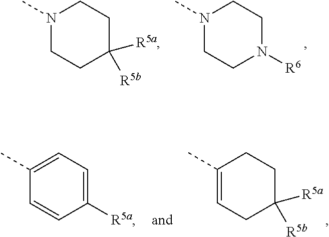

wherein:
R^{5a} and R^{5b}, if present, are each independently selected from the group consisting of C₁-C₆ alkyl, C₁-C₆ alkoxy, C₃-C₆ cycloalkyl, phenyl, thiophen-2-yl, thiophen-3-yl, and CN,
   wherein each substituent in R^{5a} and R^{5b} is optionally substituted with at CN, and
   wherein R^{5a} and R^{5b}, if present, may combine with the atoms to which they are bound to form a C₂-C₆ heterocyclyl or C₃-C₆ cycloalkyl; and
R⁶ is S(═O)₂R^{a}.

Embodiment 7 provides the method of Embodiment 6, wherein R^{5a} and R^{5b} are each independently selected from the group consisting of Me, t-Bu, 1-cyanocyclopropyl, 1-cyanocyclobutyl, 1-cyanocyclopentyl, 1-cyanocyclohexyl, phenyl, thiophen-2-yl, thiophen-3-yl, and CN.

Embodiment 8 provides the method of Embodiment 6 or 7, wherein R⁶ is ethenylsulfonyl.

Embodiment 9 provides the method of any one of Embodiments 2-8, wherein R^{1a} is selected from the group consisting of:

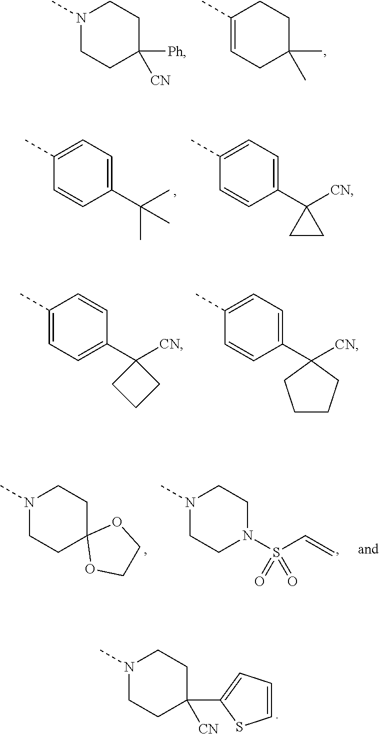

Embodiment 10 provides the method of any one of Embodiments 2-9, wherein R^{1b} is H.

Embodiment 11 provides the method of any one of Embodiments 2-10, wherein R³ is selected from the group consisting of:

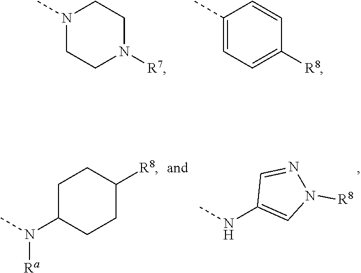

wherein:
R⁷ is selected from the group consisting of C(═O)R^{a} and S(═O)₂R^{a}; and
R⁸ is selected from the group consisting of C₁-C₆ alkoxy, C₁-C₆ hydroxyalkyl, and OH.

Embodiment 12 provides the method of Embodiment 11, wherein R is selected from the group consisting of cyclopropylcarbonyl, methylsulfonyl, dimethylaminosulfonyl, and dimethylaminocarbonyl.

Embodiment 13 provides the method of Embodiment 11 or 12, wherein R⁸ is selected from the group consisting of methoxy, 2-hydroxyethyl, and OH.

Embodiment 14 provides the method of any one of Embodiments 2-10, wherein R³ is selected from the group consisting of:

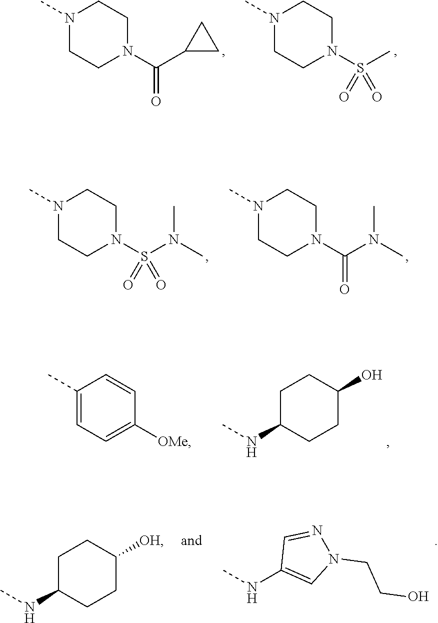

Embodiment 15 provides the method of any one of Embodiments 2-14, wherein the compound of formula (I) is selected from the group consisting of:
8-(6-methoxy-3-((4-methoxyphenyl)sulfonyl)quinolin-4-yl)-1,4-dioxa-8-azaspiro[4.5]decane;
1-(3-(4-(cyclopropanecarbonyl)piperazine-1-carbonyl)-6-fluoroquinolin-4-yl)-4-phenylpiperidine-4-carbonitrile;
(4-(cyclopropanecarbonyl)piperazin-1-yl)(4-(4,4-dimethylcyclohex-1-en-1-yl)-6-fluoroquinolin-3-yl)methanone;
(4-(4-(tert-butyl)phenyl)-6-fluoroquinolin-3-yl)(4-(cyclopropanecarbonyl)piperazin-1-yl)methanone;
1-(4-(3-(4-(cyclopropanecarbonyl)piperazine-1-carbonyl)-6-fluoroquinolin-4-yl)phenyl)cyclopropane-1-carbonitrile;
1-(6-fluoro-3-(4-(methylsulfonyl)piperazine-1-carbonyl)quinolin-4-yl)-4-phenylpiperidine-4-carbonitrile;
(4-(4,4-dimethylcyclohex-1-en-1-yl)-6-fluoroquinolin-3-yl)(4-(methylsulfonyl)piperazin-1-yl)methanone;
(4-(4-(tert-butyl)phenyl)-6-fluoroquinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclopropane-1-carbonitrile;
4-(4-cyano-4-phenylpiperidin-1-yl)-6-fluoro-N-((1r,4r)-4-hydroxycyclohexyl)quinoline-3-carboxamide;
(6-fluoro-4-(4-(vinylsulfonyl)piperazin-1-yl)quinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclobutane-1-carbonitrile;
1-(4-(6-fluoro-3-(4-(methylsulfonyl)piperazine-1-carbonyl)quinolin-4-yl)phenyl)cyclopentane-1-carbonitrile;
4-(4-(4-cyano-4-phenylpiperidin-1-yl)-6-fluoroquinoline-3-carbonyl)-N,N-dimethylpiperazine-1-sulfonamide;
4-(4-(4-cyano-4-phenylpiperidin-1-yl)-6-fluoroquinoline-3-carbonyl)-N,N-dimethylpiperazine-1-carboxamide;
1-(6-chloro-3-(4-(methylsulfonyl)piperazine-1-carbonyl)quinolin-4-yl)-4-phenylpiperidine-4-carbonitrile;
1-(6-chloro-3-(4-(cyclopropanecarbonyl)piperazine-1-carbonyl)quinolin-4-yl)-4-phenylpiperidine-4-carbonitrile;
1-(6-(4-(methylsulfonyl)piperazine-1-carbonyl)thieno[3,2-b]pyridin-7-yl)-4-phenylpiperidine-4-carbonitrile;
1-(6-(4-(cyclopropanecarbonyl)piperazine-1-carbonyl)thieno[3,2-b]pyridin-7-yl)-4-phenylpiperidine-4-carbonitrile;
1-(6-methoxy-3-(4-(methylsulfonyl)piperazine-1-carbonyl)quinolin-4-yl)-4-phenylpiperidine-4-carbonitrile;
1-(7-methoxy-3-(4-(methylsulfonyl)piperazine-1-carbonyl)quinolin-4-yl)-4-phenylpiperidine-4-carbonitrile;
1-(6,7-difluoro-3-(4-(methylsulfonyl)piperazine-1-carbonyl)quinolin-4-yl)-4-phenylpiperidine-4-carbonitrile;
1-(6,8-difluoro-3-(4-(methylsulfonyl)piperazine-1-carbonyl)quinolin-4-yl)-4-phenylpiperidine-4-carbonitrile;
4-(4-(4-(1-cyanocyclopropyl)phenyl)-6-fluoroquinoline-3-carbonyl)-N,N-dimethylpiperazine-1-sulfonamide;
4-(4-(4-(1-cyanocyclopropyl)phenyl)-6-fluoroquinoline-3-carbonyl)-N,N-dimethylpiperazine-1-carboxamide;
4-(6-chloro-4-(4-(1-cyanocyclopropyl)phenyl)quinoline-3-carbonyl)-N,N-dimethylpiperazine-1-sulfonamide;
1-(4-(6-chloro-3-(4-(cyclopropanecarbonyl)piperazine-1-carbonyl)quinolin-4-yl)phenyl)cyclopropane-1-carbonitrile;
1-(4-(6-(4-(methylsulfonyl)piperazine-1-carbonyl)thieno[3,2-b]pyridin-7-yl)phenyl)cyclopropane-1-carbonitrile;
1-(4-(6-(4-(cyclopropanecarbonyl)piperazine-1-carbonyl)thieno[3,2-b]pyridin-7-yl)phenyl)cyclopropane-1-carbonitrile;
1-(4-(6-methoxy-3-(4-(methylsulfonyl)piperazine-1-carbonyl)quinolin-4-yl)phenyl)cyclopropane-1-carbonitrile;
1-(4-(7-methoxy-3-(4-(methylsulfonyl)piperazine-1-carbonyl)quinolin-4-yl)phenyl)cyclopropane-1-carbonitrile;
1-(4-(6,7-difluoro-3-(4-(methylsulfonyl)piperazine-1-carbonyl)quinolin-4-yl)phenyl)cyclopropane-1-carbonitrile;
1-(4-(6,8-difluoro-3-(4-(methylsulfonyl)piperazine-1-carbonyl)quinolin-4-yl)phenyl)cyclopropane-1-carbonitrile;
4-(4-(1-cyanocyclopropyl)phenyl)-6-fluoro-N-((1r,4r)-4-hydroxycyclohexyl)quinoline-3-carboxamide;
4-(4-(1-cyanocyclopropyl)phenyl)-6-fluoro-N-(1-(2-hydroxyethyl)-1H-pyrazol-4-yl)quinoline-3-carboxamide; and
4-(4-cyano-4-phenylpiperidin-1-yl)-6-fluoro-N-(1-(2-hydroxyethyl)-1H-pyrazol-4-yl)quinoline-3-carboxamide.

Embodiment 16 provides the method of any one of Embodiments 1-15, wherein the solid tumor is a carcinoma.

Embodiment 17 provides the method of Embodiment 16, wherein the carcinoma comprises human hepatocellular carcinoma (HCC) cells.

Embodiment 18 provides the method of any one of Embodiments 1-17, wherein RALDH1 is overexpressed in the solid tumor.

Embodiment 19 provides the method of Embodiment 18, wherein expression of Raldh1 and Raldh2 or Raldh1 and Raldh3 in the solid tumor has a ratio selected from the group consisting of about 1000:1, 500:1, 250:1, 100:1, 50:1, 25:1, 10:1, and 5:1.

Embodiment 20 provides the method of any one of Embodiments 1-19, wherein the immunostimulator is at least one selected from the group consisting of an immune checkpoint inhibitor, chimeric antigen receptor (CAR) T-cells, T-cells engineered to express specific TCR targeted tumor antigens (TCR-transgenic), ex vivo expanded T-cells, and bispecific T-cell engagers (BiTE).

Embodiment 21 provides the method of Embodiment 20, wherein the immunostimulator is an immune checkpoint inhibitor.

Embodiment 22 provides the method of Embodiment 21, wherein the immune checkpoint inhibitor is selected from the group consisting of an anti-PD1 antibody, an anti-PD-L1 antibody, an anti-CTLA4 antibody, any fragment thereof, and any combinations thereof.

Embodiment 23 provides the method of Embodiment 20, wherein the immunostimulator is CAR T-cells.

Embodiment 24 provides the method of Embodiment 23, wherein the CAR T-cells are administered intravenously.

Embodiment 25 provides the method of Embodiment 23 or 24, wherein the CAR T-cells are administered as a CAR T-cell therapy.

Embodiment 26 provides the method of any one of Embodiments 1-25, wherein the subject is administered an immune checkpoint inhibitor and chimeric antigen receptor (CAR) T-cells.

Embodiment 27 provides the method of any one of Embodiments 1-26, further comprising administering to the subject at least one selected from the group consisting of a retinoic acid receptor (RAR) inhibitor and a retinoid X receptor (RXR) inhibitor.

Embodiment 28 provides the method of Embodiment 27, wherein the RAR inhibitor is selected from the group consisting of AGN 193109, BMS 195614, BMS 493, CD 2665, ER 50891, LE 135, LY 2955303, MM 11253, any salt or solvate thereof, and any combinations thereof.

Embodiment 29 provides the method of Embodiment 27 or 28, wherein the RXR inhibitor is selected from the group consisting of HX 531, PA 452, and UVI 3003, any salt or solvate thereof, and any combinations thereof.

Embodiment 30 provides the method of any one of Embodiments 1-29, wherein the RALDH1 inhibitor and the immunostimulator are administered to the subject simultaneously or sequentially.

Embodiment 31 provides the method of any one of Embodiments 1-30, wherein the subject is a mammal.

Embodiment 32 provides the method of Embodiment 31, wherein the mammal is a human.

Embodiment 33 provides a pharmaceutical composition comprising:
(a) at least one immunostimulator;
(b) a pharmaceutically acceptable carrier; and
(c) a retinaldehyde dehydrogenase 1 (RALDH1) inhibitor, wherein the RALDH1 inhibitor is selected from the group consisting of:
(i) a compound of formula (I), or a salt, solvate, prodrug, stereoisomer, tautomer, or

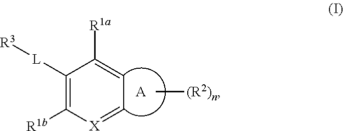

wherein:
R^{1a} is selected from the group consisting of optionally substituted C₂-C₈ heterocyclyl, optionally substituted phenyl, and optionally substituted C₅-C₈cycloalkenyl,
   wherein each optional substituent in R^{1a} is independently selected from the group consisting of C₁-C₆ alkyl, C₃-C₆ cycloalkyl, C₁-C₆ alkoxy, optionally substituted phenyl, optionally substituted C₂-C₈ heterocyclyl, halogen, OH, N(R^{a})(R), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b},
      wherein each optional substituent is optionally substituted with at least one substituent selected from the group consisting of C₁-C₃ alkyl, C₁-C₃ alkoxy, halogen, CN, and NO₂, and
   wherein two vicinal or geminal optional substituents in R^{1a} may combine with the atoms to which they are bound to form a C₂-C₈ heterocyclyl or C₃-C₈ cycloalkyl;
R^{1b} and R^{1c}, if present, are each independently selected from the group consisting of H, C₁-C₆ alkyl, C₁-C₆ alkoxy, halogen, OH, N(R^{a})(R^{b}), NO₂, and CN;
each occurrence of R² is independently selected from the group consisting of C₁-C₆ alkyl, C₁-C₆ alkoxy, C₁-C₃ haloalkoxy, C₁-C₆ hydroxyalkyl, halogen, NO₂, and CN;
R³ is selected from the group consisting of optionally substituted C₂-C₈ heterocyclyl, optionally substituted phenyl, N(R^{a})(optionally substituted C₃-C₈ cycloalkyl), and N(R^{a})(optionally substituted C₂-C₈ heterocyclyl),
   wherein each optional substituent in R³ is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b};
A is selected from the group consisting of optionally substituted C₆-C₁₀ aryl and optionally substituted C₂-C₈ heterocyclyl
   wherein each optional substituent in A is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b}.
L is selected from the group consisting of —CH₂—, —C(═O)—, and —S(═O)₂—;
X is selected from the group consisting of N and CR^{1c};
n is an integer selected from the group consisting of 0, 1, 2, 3, and 4; and
each occurrence of R^{a}, R^{b}, and R^{c} is independently selected from the group consisting of H, C₁-C₆ alkyl, C₂-C₆ alkenyl, C₂-C₆ alkynyl, C₃-C₈ cycloalkyl, and C₂-C₈ heterocyclyl; and
(ii) a compound selected from the group consisting of:
   1-benzylindoline-2,3-dione;
   ethyl 2-((4-oxo-3-(3-(pyrrolidin-1-yl)propyl)-3,4-dihydrobenzo[4,5]thieno[3,2-d]pyrimidin-2-yl)thio)acetate;
   2-((2-(sec-butyl)-3-oxo-2,3-dihydroimidazo[1,2-c]quinazolin-5-yl)thio)-N-(o-tolyl)butanamide; and
   8-((4-(cyclopropanecarbonyl)piperazin-1-yl)methyl)-7-isopentyl-1,3-dimethyl-3,7-dihydro-1H-purine-2,6-dione;
   or a salt, solvate, prodrug, stereoisomer, tautomer, or isotopologue thereof.

Embodiment 34 provides the pharmaceutical composition of Embodiment 33, wherein the RALDH1 inhibitor is a compound of formula (I).

Embodiment 35 provides the pharmaceutical composition of Embodiment 34, wherein the compound of formula (I) is selected from the group consisting of:
8-(6-methoxy-3-((4-methoxyphenyl)sulfonyl)quinolin-4-yl)-1,4-dioxa-8-azaspiro[4.5]decane;
1-(3-(4-(cyclopropanecarbonyl)piperazine-1-carbonyl)-6-fluoroquinolin-4-yl)-4-phenylpiperidine-4-carbonitrile,
(4-(cyclopropanecarbonyl)piperazin-1-yl)(4-(4,4-dimethylcyclohex-1-en-1-yl)-6-fluoroquinolin-3-yl)methanone;
(4-(4-(tert-butyl)phenyl)-6-fluoroquinolin-3-yl)(4-(cyclopropanecarbonyl)piperazin-1-yl)methanone;
1-(4-(3-(4-(cyclopropanecarbonyl)piperazine-1-carbonyl)-6-fluoroquinolin-4-yl)phenyl)cyclopropane-1-carbonitrile;
1-(6-fluoro-3-(4-(methylsulfonyl)piperazine-1-carbonyl)quinolin-4-yl)-4-phenylpiperidine-4-carbonitrile;
(4-(4,4-dimethylcyclohex-1-en-1-yl)-6-fluoroquinolin-3-yl)(4-(methylsulfonyl)piperazin-1-yl)methanone;
(4-(4-(tert-butyl)phenyl)-6-fluoroquinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclopropane-1-carbonitrile;
4-(4-cyano-4-phenylpiperidin-1-yl)-6-fluoro-N-((1r,4r)-4-hydroxycyclohexyl)quinoline-3-carboxamide;
(6-fluoro-4-(4-(vinylsulfonyl)piperazin-1-yl)quinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclobutane-1-carbonitrile;
1-(4-(6-fluoro-3-(4-(methylsulfonyl)piperazine-1-carbonyl)quinolin-4-yl)phenyl)cyclopentane-1-carbonitrile;
4-(4-(4-cyano-4-phenylpiperidin-1-yl)-6-fluoroquinoline-3-carbonyl)-N,N-dimethylpiperazine-1-sulfonamide;
4-(4-(4-cyano-4-phenylpiperidin-1-yl)-6-fluoroquinoline-3-carbonyl)-N,N-dimethylpiperazine-1-carboxamide;
1-(6-chloro-3-(4-(methylsulfonyl)piperazine-1-carbonyl)quinolin-4-yl)-4-phenylpiperidine-4-carbonitrile;
1-(6-chloro-3-(4-(cyclopropanecarbonyl)piperazine-1-carbonyl)quinolin-4-yl)-4-phenylpiperidine-4-carbonitrile;
1-(6-(4-(methylsulfonyl)piperazine-1-carbonyl)thieno[3,2-b]pyridin-7-yl)-4-phenylpiperidine-4-carbonitrile;
1-(6-(4-(cyclopropanecarbonyl)piperazine-1-carbonyl)thieno[3,2-b]pyridin-7-yl)-4-phenylpiperidine-4-carbonitrile;
1-(6-methoxy-3-(4-(methylsulfonyl)piperazine-1-carbonyl)quinolin-4-yl)-4-phenylpiperidine-4-carbonitrile;
1-(7-methoxy-3-(4-(methylsulfonyl)piperazine-1-carbonyl)quinolin-4-yl)-4-phenylpiperidine-4-carbonitrile;
1-(6,7-difluoro-3-(4-(methylsulfonyl)piperazine-1-carbonyl)quinolin-4-yl)-4-phenylpiperidine-4-carbonitrile;
1-(6,8-difluoro-3-(4-(methylsulfonyl)piperazine-1-carbonyl)quinolin-4-yl)-4-phenylpiperidine-4-carbonitrile;
4-(4-(4-(1-cyanocyclopropyl)phenyl)-6-fluoroquinoline-3-carbonyl)-N,N-dimethylpiperazine-1-sulfonamide;
4-(4-(4-(1-cyanocyclopropyl)phenyl)-6-fluoroquinoline-3-carbonyl)-N,N-dimethylpiperazine-1-carboxamide;
4-(6-chloro-4-(4-(1-cyanocyclopropyl)phenyl)quinoline-3-carbonyl)-N,N-dimethylpiperazine-1-sulfonamide;
1-(4-(6-chloro-3-(4-(cyclopropanecarbonyl)piperazine-1-carbonyl)quinolin-4-yl)phenyl)cyclopropane-1-carbonitrile;
1-(4-(6-(4-(methylsulfonyl)piperazine-1-carbonyl)thieno[3,2-b]pyridin-7-yl)phenyl)cyclopropane-1-carbonitrile;
1-(4-(6-(4-(cyclopropanecarbonyl)piperazine-1-carbonyl)thieno[3,2-b]pyridin-7-yl)phenyl)cyclopropane-1-carbonitrile;
1-(4-(6-methoxy-3-(4-(methylsulfonyl)piperazine-1-carbonyl)quinolin-4-yl)phenyl)cyclopropane-1-carbonitrile;
1-(4-(7-methoxy-3-(4-(methylsulfonyl)piperazine-1-carbonyl)quinolin-4-yl)phenyl)cyclopropane-1-carbonitrile
1-(4-(6,7-difluoro-3-(4-(methylsulfonyl)piperazine-1-carbonyl)quinolin-4-yl)phenyl)cyclopropane-1-carbonitrile;
1-(4-(6,8-difluoro-3-(4-(methylsulfonyl)piperazine-1-carbonyl)quinolin-4-yl)phenyl)cyclopropane-1-carbonitrile;
4-(4-(1-cyanocyclopropyl)phenyl)-6-fluoro-N-((1r,4r)-4-hydroxycyclohexyl)quinoline-3-carboxamide;
4-(4-(1-cyanocyclopropyl)phenyl)-6-fluoro-N-(1-(2-hydroxyethyl)-1H-pyrazol-4-yl)quinoline-3-carboxamide; and
4-(4-cyano-4-phenylpiperidin-1-yl)-6-fluoro-N-(1-(2-hydroxyethyl)-1H-pyrazol-4-yl)quinoline-3-carboxamide.

Embodiment 36 provides the pharmaceutical composition of any one of Embodiments 33-35, wherein the immunostimulator is at least one selected from the group consisting of an immune checkpoint inhibitor, chimeric antigen receptor (CAR) T-cells, T-cells engineered to express specific TCR targeted tumor antigens (TCR-transgenic), ex vivo expanded T-cells, and bispecific T-cell engagers (BiTE).

Embodiment 37 provides the pharmaceutical composition of Embodiment 36, wherein the immunostimulator is an immune checkpoint inhibitor.

Embodiment 38 provides the pharmaceutical composition of Embodiment 37, wherein the immune checkpoint inhibitor is selected from the group consisting of an anti-PD1 antibody, an anti-PD-L1 antibody, an anti-CTLA4 antibody, any fragment thereof, and any combinations thereof.

Embodiment 39 provides the pharmaceutical composition of any one of Embodiments 33-35, wherein the immunostimulator is CAR T-cells.

Embodiment 40 provides the pharmaceutical composition of any one of Embodiments 33-39, wherein the at least one immunostimulator comprises an immune checkpoint inhibitor and CAR T-cells.

Embodiment 41 provides the pharmaceutical composition of any one of Embodiments 33-40, further comprising at least one selected from the group consisting of a retinoic acid receptor (RAR) inhibitor and a retinoid X receptor (RXR) inhibitor.

Embodiment 42 provides the pharmaceutical composition of Embodiment 41, wherein the RAR inhibitor is selected from the group consisting of AGN 193109, BMS 195614, BMS 493, CD 2665, ER 50891, LE 135, LY 2955303, MM 11253, any salt or solvate thereof, and any combinations thereof.

Embodiment 43 provides the pharmaceutical composition of Embodiment 41 or 42, wherein the RXR inhibitor is selected from the group consisting of HX 531, PA 452, and UVI 3003, any salt or solvate thereof, and any combinations thereof.

Embodiment 44 provides the pharmaceutical composition of any one of Embodiments 33-43, wherein the pharmaceutically acceptable carrier is suitable for intravenous administration.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating, preventing, and/or ameliorating a solid tumor in a subject in need thereof, the method comprising administering to the subject a pharmaceutically effective amount of:
(a) at least one immunostimulator; and
(b) a retinaldehyde dehydrogenase 1 (RALDH1) inhibitor, wherein the RALDH1 inhibitor is selected from the group consisting of:
(i) a compound of formula (I), or a salt, solvate, prodrug, stereoisomer, tautomer, or isotopologue thereof: 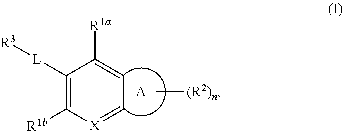
wherein:
R^{1a} is selected from the group consisting of optionally substituted C₂-C₈ heterocyclyl, optionally substituted phenyl, and optionally substituted C₅-C₈ cycloalkenyl,
wherein each optional substituent in R^{1a} is independently selected from the group consisting of C₁-C₆ alkyl, C₃-C₆ cycloalkyl, C₁-C₆ alkoxy, optionally substituted phenyl, optionally substituted C₂-C₈ heterocyclyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b},
wherein each optional substituent is optionally substituted with at least one substituent selected from the group consisting of C₁-C₃ alkyl, C₁-C₃ alkoxy, halogen, CN, and NO₂, and
wherein two vicinal or geminal optional substituents in R^{1a} may combine with the atoms to which they are bound to form a C₂-C₈ heterocyclyl or C₃-C₈ cycloalkyl;
R^{1b} and R^{1c}, if present, are each independently selected from the group consisting of H, C₁-C₆ alkyl, C₁-C₆ alkoxy, halogen, OH, N(R^{a})(R^{b}), NO₂, and CN;
each occurrence of R² is independently selected from the group consisting of C₁-C₆ alkyl, C₁-C₆ alkoxy, C₁-C₃ haloalkoxy, C₁-C₆ hydroxyalkyl, halogen, NO₂, and CN;
R³ is selected from the group consisting of optionally substituted C₂-C₈ heterocyclyl, optionally substituted phenyl, N(R^{a})(optionally substituted C₃-C₈ cycloalkyl), and N(R^{a})(optionally substituted C₂-C₈ heterocyclyl),
wherein each optional substituent in R³ is independently selected from the group consisting of C₁-C₆ alkyl, C₁-C₆ alkoxy, C₁-C₆ hydroxyalkyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b};
A is selected from the group consisting of optionally substituted C₆-C₁₀ aryl and optionally substituted C₂-C₈ heterocyclyl,
wherein each optional substituent in A is independently selected from the group consisting of C₁-C₆ alkyl, C₁-C₆ alkoxy, C₁-C₆ hydroxyalkyl, halogen, OH, N(R^{a})(R), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b};
L is selected from the group consisting of —CH₂—, —C(═O)—, and —S(═O)₂—;
X is selected from the group consisting of N and CR^{1c};
n is an integer selected from the group consisting of 0, 1, 2, 3, and 4; and
each occurrence of R^{a}, R^{b}, and R^{c} is independently selected from the group consisting of H, C₁-C₆ alkyl, C₂-C₆ alkenyl, C₂-C₆ alkynyl, C₃-C₈ cycloalkyl, and C₂-C₈ heterocyclyl; and
(ii) a compound selected from the group consisting of:
1-benzylindoline-2,3-dione;
ethyl 2-((4-oxo-3-(3-(pyrrolidin-1-yl)propyl)-3,4-dihydrobenzo[4,5]thieno[3,2-d]pyrimidin-2-yl)thio)acetate;
2-((2-(sec-butyl)-3-oxo-2,3-dihydroimidazo[1,2-c]quinazolin-5-yl)thio)-N-(o-tolyl)butanamide; and
8-((4-(cyclopropanecarbonyl)piperazin-1-yl)methyl)-7-isopentyl-1,3-dimethyl-3,7-dihydro-1H-purine-2,6-dione;
or a salt, solvate, prodrug, stereoisomer, tautomer, or isotopologue thereof.

2. (canceled)

3. The method of claim 1, wherein the RALDH1 inhibitor is a compound of formula (I) selected from the group consisting of: 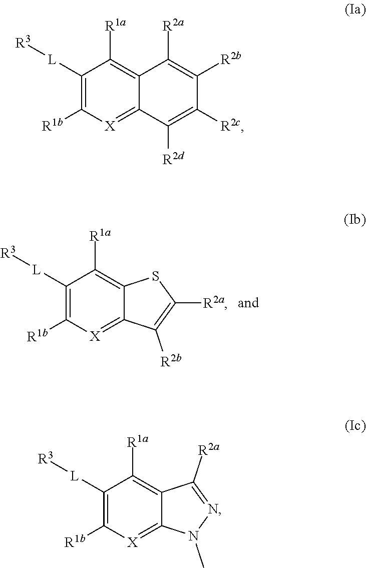
wherein:
R^{2a}, R^{2b}, R^{2c}, and R^{2d}, if present, are each independently selected from the group consisting of H, C₁-C₆ alkyl, C₁-C₆ alkoxy, and halogen.

4. The method of claim 3, wherein at least one of the following applies:
(a) R^{2a}, R^{2b}, R^{2c}, and R^{2d}, if present, are each independently selected from the group consisting of H, Me, OMe, F, and Cl;
(b) X is N;
(c) R^{1a} is selected from the group consisting of: 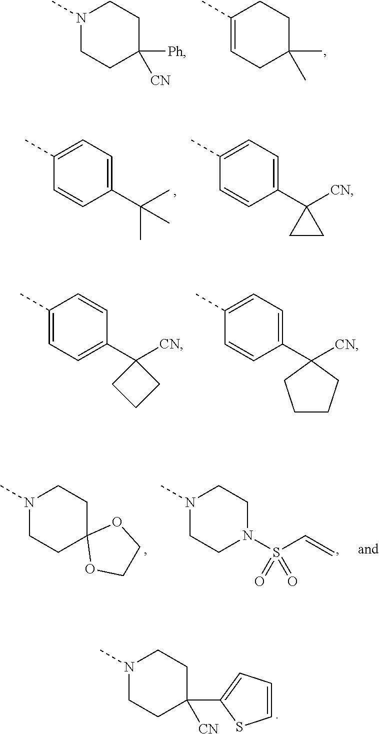
(d) R^{1b} is H; and
(e) R³ is selected from the group consisting of:

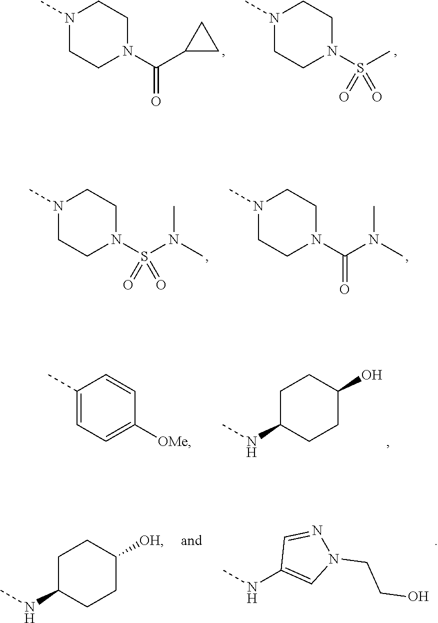

5. (canceled)

6. The method of claim 3, wherein R^{1a} is selected from the group consisting of: 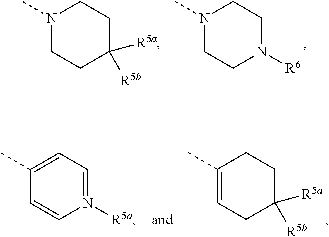
wherein:
R^{5a} and R^{5b}, if present, are each independently selected from the group consisting of C₁-C₆ alkyl, C₁-C₆ alkoxy, C₃-C₆ cycloalkyl, phenyl, thiophen-2-yl, thiophen-3-yl, and CN,
wherein each substituent in R^{5a} and R^{5b} is optionally substituted with at CN, and
wherein R^{5a} and R^{5b}, if present, may combine with the atoms to which they are bound to form a C₂-C₆ heterocyclyl or C₃-C₆ cycloalkyl; and
R⁶ is S(═O)₂R^{a}.

7. The method of claim 6, wherein at least one of the following applies:
(a) R^{5a} and R^{5b} are each independently selected from the group consisting of Me, t-Bu, 1-cyanocyclopropyl, 1-cyanocyclobutyl, 1-cyanocyclopentyl, 1-cyanocyclohexyl, phenyl, thiophen-2-yl, thiophen-3-yl, and CN; and
(b) R⁶ is ethenylsulfonyl.

8. (canceled)

9. (canceled)

10. (canceled)

11. The method of claim 3, wherein R³ is selected from the group consisting of: 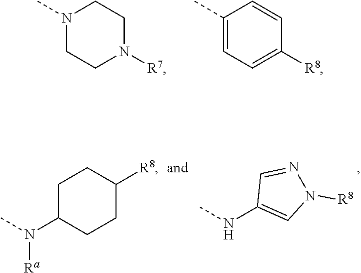
wherein:
R⁷ is selected from the group consisting of C(═O)R^{a} and S(═O)₂R^{a}; and
R⁸ is selected from the group consisting of C₁-C₆ alkoxy, C₁-C₆ hydroxyalkyl, and OH.

12. The method of claim 11, wherein at least one of the following applies:
(a) R⁷ is selected from the group consisting of cyclopropylcarbonyl, methylsulfonyl, dimethylaminosulfonyl, and dimethylaminocarbonyl; and
(b) R⁸ is selected from the group consisting of methoxy, 2-hydroxyethyl, and OH.

13. (canceled)

14. The method of claim 3, wherein R³ is selected from the group consisting of:

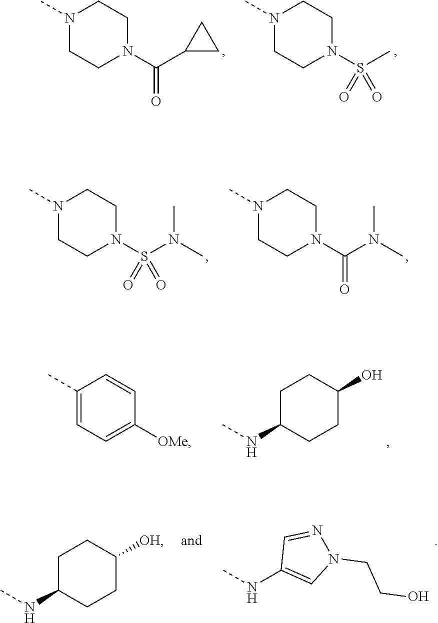

15. The method of claim 3, wherein the compound of formula (I) is selected from the group consisting of:
8-(6-methoxy-3-((4-methoxyphenyl)sulfonyl)quinolin-4-yl)-1,4-dioxa-8-azaspiro[4.5]decane;
1-(3-(4-(cyclopropanecarbonyl)piperazine-1-carbonyl)-6-fluoroquinolin-4-yl)-4-phenylpiperidine-4-carbonitrile;
(4-(cyclopropanecarbonyl)piperazin-1-yl)(4-(4,4-dimethylcyclohex-1-en-1-yl)-6-fluoroquinolin-3-yl)methanone;
(4-(4-(tert-butyl)phenyl)-6-fluoroquinolin-3-yl)(4-(cyclopropanecarbonyl)piperazin-1-yl)methanone;
1-(4-(3-(4-(cyclopropanecarbonyl)piperazine-1-carbonyl)-6-fluoroquinolin-4-yl)phenyl)cyclopropane-1-carbonitrile;
1-(6-fluoro-3-(4-(methylsulfonyl)piperazine-1-carbonyl)quinolin-4-yl)-4-phenylpiperidine-4-carbonitrile;
(4-(4,4-dimethylcyclohex-1-en-1-yl)-6-fluoroquinolin-3-yl)(4-(methylsulfonyl)piperazin-1-yl)methanone;
(4-(4-(tert-butyl)phenyl)-6-fluoroquinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclopropane-1-carbonitrile;
4-(4-cyano-4-phenylpiperidin-1-yl)-6-fluoro-N-((1r,4r)-4-hydroxycyclohexyl)quinoline-3-carboxamide;
(6-fluoro-4-(4-(vinylsulfonyl)piperazin-1-yl)quinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclobutane-1-carbonitrile;
1-(4-(6-fluoro-3-(4-(methylsulfonyl)piperazine-1-carbonyl)quinolin-4-yl)phenyl)cyclopentane-1-carbonitrile;
4-(4-(4-cyano-4-phenylpiperidin-1-yl)-6-fluoroquinoline-3-carbonyl)-N,N-dimethylpiperazine-1-sulfonamide;
4-(4-(4-cyano-4-phenylpiperidin-1-yl)-6-fluoroquinoline-3-carbonyl)-N,N-dimethylpiperazine-1-carboxamide;
1-(6-chloro-3-(4-(methylsulfonyl)piperazine-1-carbonyl)quinolin-4-yl)-4-phenylpiperidine-4-carbonitrile;
1-(6-chloro-3-(4-(cyclopropanecarbonyl)piperazine-1-carbonyl)quinolin-4-yl)-4-phenylpiperidine-4-carbonitrile;
1-(6-(4-(methylsulfonyl)piperazine-1-carbonyl)thieno[3,2-b]pyridin-7-yl)-4-phenylpiperidine-4-carbonitrile;
1-(6-(4-(cyclopropanecarbonyl)piperazine-1-carbonyl)thieno[3,2-b]pyridin-7-yl)-4-phenylpiperidine-4-carbonitrile;
1-(6-methoxy-3-(4-(methylsulfonyl)piperazine-1-carbonyl)quinolin-4-yl)-4-phenylpiperidine-4-carbonitrile;
1-(7-methoxy-3-(4-(methylsulfonyl)piperazine-1-carbonyl)quinolin-4-yl)-4-phenylpiperidine-4-carbonitrile;
1-(6,7-difluoro-3-(4-(methylsulfonyl)piperazine-1-carbonyl)quinolin-4-yl)-4-phenylpiperidine-4-carbonitrile;
1-(6,8-difluoro-3-(4-(methylsulfonyl)piperazine-1-carbonyl)quinolin-4-yl)-4-phenylpiperidine-4-carbonitrile;
4-(4-(4-(1-cyanocyclopropyl)phenyl)-6-fluoroquinoline-3-carbonyl)-N,N-dimethylpiperazine-1-sulfonamide;
4-(4-(4-(1-cyanocyclopropyl)phenyl)-6-fluoroquinoline-3-carbonyl)-N,N-dimethylpiperazine-1-carboxamide;
4-(6-chloro-4-(4-(1-cyanocyclopropyl)phenyl)quinoline-3-carbonyl)-N,N-dimethylpiperazine-1-sulfonamide;
1-(4-(6-chloro-3-(4-(cyclopropanecarbonyl)piperazine-1-carbonyl)quinolin-4-yl)phenyl)cyclopropane-1-carbonitrile;
1-(4-(6-(4-(methylsulfonyl)piperazine-1-carbonyl)thieno[3,2-b]pyridin-7-yl)phenyl)cyclopropane-1-carbonitrile;
1-(4-(6-(4-(cyclopropanecarbonyl)piperazine-1-carbonyl)thieno[3,2-b]pyridin-7-yl)phenyl)cyclopropane-1-carbonitrile;
1-(4-(6-methoxy-3-(4-(methylsulfonyl)piperazine-1-carbonyl)quinolin-4-yl)phenyl)cyclopropane-1-carbonitrile;
1-(4-(7-methoxy-3-(4-(methylsulfonyl)piperazine-1-carbonyl)quinolin-4-yl)phenyl)cyclopropane-1-carbonitrile;
1-(4-(6,7-difluoro-3-(4-(methylsulfonyl)piperazine-1-carbonyl)quinolin-4-yl)phenyl)cyclopropane-1-carbonitrile;
1-(4-(6,8-difluoro-3-(4-(methylsulfonyl)piperazine-1-carbonyl)quinolin-4-yl)phenyl)cyclopropane-1-carbonitrile;
4-(4-(1-cyanocyclopropyl)phenyl)-6-fluoro-N-((1r,4r)-4-hydroxycyclohexyl)quinoline-3-carboxamide;
4-(4-(1-cyanocyclopropyl)phenyl)-6-fluoro-N-(1-(2-hydroxyethyl)-1H-pyrazol-4-yl)quinoline-3-carboxamide; and
4-(4-cyano-4-phenylpiperidin-1-yl)-6-fluoro-N-(1-(2-hydroxyethyl)-1H-pyrazol-4-yl)quinoline-3-carboxamide.

16. The method of claim 1, wherein at least one of the following applies:
(a) the solid tumor is a carcinoma, optionally wherein the carcinoma comprises human hepatocellular carcinoma (HCC) cells; and
(b) RALDH1 is overexpressed in the solid tumor, optionally wherein expression of Raldh1 and Raldh2 or Raldh1 and Raldh3 in the solid tumor has a ratio selected from the group consisting of about 1000:1, 500:1, 250:1, 100:1, 50:1, 25:1, 10:1, and 5:1.

17. (canceled)

18. (canceled)

19. (canceled)

20. The method of claim 1, wherein the immunostimulator is at least one selected from the group consisting of an immune checkpoint inhibitor, chimeric antigen receptor (CAR) T-cells, T-cells engineered to express specific TCR targeted tumor antigens (TCR-transgenic), ex vivo expanded T-cells, and bispecific T-cell engagers (BiTE).

21. The method of claim 20, wherein at least one of the following applies:
(a) the immunostimulator is an immune checkpoint inhibitor, optionally wherein the immune checkpoint inhibitor is selected from the group consisting of an anti-PD1 antibody, an anti-PD-L1 antibody, an anti-CTLA4 antibody, any fragment thereof, and any combinations thereof;
(b) the immunostimulator is CAR T-cells, optionally wherein the CAR T-cells are administered intravenously, and optionally wherein the CAR T-cells are administered as a CAR T-cell therapy; and
(c) the subject is administered an immune checkpoint inhibitor and chimeric antigen receptor (CAR) T-cells.

22. (canceled)

23. (canceled)

24. (canceled)

25. (canceled)

26. (canceled)

27. The method of claim 1, further comprising administering to the subject at least one selected from the group consisting of a retinoic acid receptor (RAR) inhibitor and a retinoid X receptor (RXR) inhibitor.

28. The method of claim 27, wherein at least one of the following applies:
(a) the RAR inhibitor is selected from the group consisting of AGN 193109, BMS 195614, BMS 493, CD 2665, ER 50891, LE 135, LY 2955303, MM 11253, any salt or solvate thereof, and any combinations thereof; and
(b) the RXR inhibitor is selected from the group consisting of HX 531, PA 452, and UVI 3003, any salt or solvate thereof, and any combinations thereof.

29. (canceled)

30. The method of claim 1, wherein the RALDH1 inhibitor and the immunostimulator are administered to the subject simultaneously or sequentially.

31. (canceled)

32. (canceled)

33. A pharmaceutical composition comprising:
(a) at least one immunostimulator;
(b) a pharmaceutically acceptable carrier; and
(c) a retinaldehyde dehydrogenase 1 (RALDH1) inhibitor, wherein the RALDH1 inhibitor is selected from the group consisting of:
(i) a compound of formula (I), or a salt, solvate, prodrug, stereoisomer, tautomer, or isotopologue thereof: 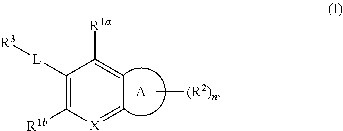
wherein:
R^{1a} is selected from the group consisting of optionally substituted C₂-C₈ heterocyclyl, optionally substituted phenyl, and optionally substituted C₅-C₈ cycloalkenyl,
wherein each optional substituent in R^{1a} is independently selected from the group consisting of C₁-C₆ alkyl, C₃-C₆ cycloalkyl, C₁-C₆ alkoxy, optionally substituted phenyl, optionally substituted C₂-C₈ heterocyclyl, halogen, OH, N(R^{a})(R^{b}), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b},
wherein each optional substituent is optionally substituted with at least one substituent selected from the group consisting of C₁-C₃ alkyl, C₁-C₃ alkoxy, halogen, CN, and NO₂, and
wherein two vicinal or geminal optional substituents in R^{1a} may combine with the atoms to which they are bound to form a C₂-C₈ heterocyclyl or C₃-C₈ cycloalkyl;
R^{1b} and R^{1c}, if present, are each independently selected from the group consisting of H, C₁-C₆ alkyl, C₁-C₆ alkoxy, halogen, OH, N(R^{a})(R^{b}), NO₂, and CN;
each occurrence of R² is independently selected from the group consisting of C₁-C₆ alkyl, C₁-C₆ alkoxy, C₁-C₃ haloalkoxy, C₁-C₆ hydroxyalkyl, halogen, NO₂, and CN;
R³ is selected from the group consisting of optionally substituted C₂-C₈ heterocyclyl, optionally substituted phenyl, N(R^{a})(optionally substituted C₃-C₈ cycloalkyl), and N(R^{a})(optionally substituted C₂-C₈ heterocyclyl),
wherein each optional substituent in R³ is independently selected from the group consisting of C₁-C₆ alkyl, C₁-C₆ alkoxy, C₁-C₆ hydroxyalkyl, halogen, OH, N(R^{a})(R), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b};
A is selected from the group consisting of optionally substituted C₆-C₁₀ aryl and optionally substituted C₂-C₈ heterocyclyl
wherein each optional substituent in A is independently selected from the group consisting of C₁-C₆ alkyl, C₁-C₆ alkoxy, C₁-C₆ hydroxyalkyl, halogen, OH, N(R^{a})(R), NO₂, CN, C(═O)R^{a}, C(═O)N(R^{a})(R^{b}), S(═O)₂R^{a}, S(═O)₂N(R^{a})(R^{b}), and N(R^{a})C(═O)R^{b};
L is selected from the group consisting of —CH₂—, —C(═O)—, and —S(═O)₂—;
X is selected from the group consisting of N and CR^{1c};
n is an integer selected from the group consisting of 0, 1, 2, 3, and 4; and
each occurrence of R^{a}, R^{b}, and R^{c} is independently selected from the group consisting of H, C₁-C₆ alkyl, C₂-C₆ alkenyl, C₂-C₆ alkynyl, C₃-C₈ cycloalkyl, and C₂-C₈ heterocyclyl; and
(ii) a compound selected from the group consisting of:
1-benzylindoline-2,3-dione;
ethyl 2-((4-oxo-3-(3-(pyrrolidin-1-yl)propyl)-3,4-dihydrobenzo[4,5]thieno[3,2-d]pyrimidin-2-yl)thio)acetate;
2-((2-(sec-butyl)-3-oxo-2,3-dihydroimidazo[1,2-c]quinazolin-5-yl)thio)-N-(o-tolyl)butanamide; and
8-((4-(cyclopropanecarbonyl)piperazin-1-yl)methyl)-7-isopentyl-1,3-dimethyl-3,7-dihydro-1H-purine-2,6-dione;
or a salt, solvate, prodrug, stereoisomer, tautomer, or isotopologue thereof.

34. (canceled)

35. The pharmaceutical composition of claim 33, wherein the RALDH1 inhibitor is a compound of formula (I), optionally wherein the compound of formula (I) is selected from the group consisting of:
8-(6-methoxy-3-((4-methoxyphenyl)sulfonyl)quinolin-4-yl)-1,4-dioxa-8-azaspiro[4.5]decane;
1-(3-(4-(cyclopropanecarbonyl)piperazine-1-carbonyl)-6-fluoroquinolin-4-yl)-4-phenylpiperidine-4-carbonitrile;
(4-(cyclopropanecarbonyl)piperazin-1-yl)(4-(4,4-dimethylcyclohex-1-en-1-yl)-6-fluoroquinolin-3-yl)methanone;
(4-(4-(tert-butyl)phenyl)-6-fluoroquinolin-3-yl)(4-(cyclopropanecarbonyl)piperazin-1-yl)methanone;
1-(4-(3-(4-(cyclopropanecarbonyl)piperazine-1-carbonyl)-6-fluoroquinolin-4-yl)phenyl)cyclopropane-1-carbonitrile;
1-(6-fluoro-3-(4-(methylsulfonyl)piperazine-1-carbonyl)quinolin-4-yl)-4-phenylpiperidine-4-carbonitrile;
(4-(4,4-dimethylcyclohex-1-en-1-yl)-6-fluoroquinolin-3-yl)(4-(methylsulfonyl)piperazin-1-yl)methanone;
(4-(4-(tert-butyl)phenyl)-6-fluoroquinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclopropane-1-carbonitrile;
4-(4-cyano-4-phenylpiperidin-1-yl)-6-fluoro-N-((1r,4r)-4-hydroxycyclohexyl)quinoline-3-carboxamide;
(6-fluoro-4-(4-(vinylsulfonyl)piperazin-1-yl)quinolin-3-yl)(4-(methylsulfonyl)piperazin-1-yl)methanone;
1-(4-(6-fluoro-3-(4-(methylsulfonyl)piperazine-1-carbonyl)quinolin-4-yl)phenyl)cyclobutane-1-carbonitrile;
1-(4-(6-fluoro-3-(4-(methylsulfonyl)piperazine-1-carbonyl)quinolin-4-yl)phenyl)cyclopentane-1-carbonitrile;
4-(4-(4-cyano-4-phenylpiperidin-1-yl)-6-fluoroquinoline-3-carbonyl)-N,N-dimethylpiperazine-1-sulfonamide;
4-(4-(4-cyano-4-phenylpiperidin-1-yl)-6-fluoroquinoline-3-carbonyl)-N,N-dimethylpiperazine-1-carboxamide;
1-(6-chloro-3-(4-(methylsulfonyl)piperazine-1-carbonyl)quinolin-4-yl)-4-phenylpiperidine-4-carbonitrile;
1-(6-chloro-3-(4-(cyclopropanecarbonyl)piperazine-1-carbonyl)quinolin-4-yl)-4-phenylpiperidine-4-carbonitrile;
1-(6-(4-(methylsulfonyl)piperazine-1-carbonyl)thieno[3,2-b]pyridin-7-yl)-4-phenylpiperidine-4-carbonitrile;
1-(6-(4-(cyclopropanecarbonyl)piperazine-1-carbonyl)thieno[3,2-b]pyridin-7-yl)-4-phenylpiperidine-4-carbonitrile;
1-(6-methoxy-3-(4-(methylsulfonyl)piperazine-1-carbonyl)quinolin-4-yl)-4-phenylpiperidine-4-carbonitrile;
1-(7-methoxy-3-(4-(methylsulfonyl)piperazine-1-carbonyl)quinolin-4-yl)-4-phenylpiperidine-4-carbonitrile;
1-(6,7-difluoro-3-(4-(methylsulfonyl)piperazine-1-carbonyl)quinolin-4-yl)-4-phenylpiperidine-4-carbonitrile;
1-(6,8-difluoro-3-(4-(methylsulfonyl)piperazine-1-carbonyl)quinolin-4-yl)-4-phenylpiperidine-4-carbonitrile;
4-(4-(4-(1-cyanocyclopropyl)phenyl)-6-fluoroquinoline-3-carbonyl)-N,N-dimethylpiperazine-1-sulfonamide;
4-(4-(4-(1-cyanocyclopropyl)phenyl)-6-fluoroquinoline-3-carbonyl)-N,N-dimethylpiperazine-1-carboxamide;
4-(6-chloro-4-(4-(1-cyanocyclopropyl)phenyl)quinoline-3-carbonyl)-N,N-dimethylpiperazine-1-sulfonamide;
1-(4-(6-chloro-3-(4-(cyclopropanecarbonyl)piperazine-1-carbonyl)quinolin-4-yl)phenyl)cyclopropane-1-carbonitrile;
1-(4-(6-(4-(methylsulfonyl)piperazine-1-carbonyl)thieno[3,2-b]pyridin-7-yl)phenyl)cyclopropane-1-carbonitrile;
1-(4-(6-(4-(cyclopropanecarbonyl)piperazine-1-carbonyl)thieno[3,2-b]pyridin-7-yl)phenyl)cyclopropane-1-carbonitrile;
1-(4-(6-methoxy-3-(4-(methylsulfonyl)piperazine-1-carbonyl)quinolin-4-yl)phenyl)cyclopropane-1-carbonitrile;
1-(4-(7-methoxy-3-(4-(methylsulfonyl)piperazine-1-carbonyl)quinolin-4-yl)phenyl)cyclopropane-1-carbonitrile
1-(4-(6,7-difluoro-3-(4-(methylsulfonyl)piperazine-1-carbonyl)quinolin-4-yl)phenyl)cyclopropane-1-carbonitrile;
1-(4-(6,8-difluoro-3-(4-(methylsulfonyl)piperazine-1-carbonyl)quinolin-4-yl)phenyl)cyclopropane-1-carbonitrile;
4-(4-(1-cyanocyclopropyl)phenyl)-6-fluoro-N-((1r,4r)-4-hydroxycyclohexyl)quinoline-3-carboxamide;
4-(4-(1-cyanocyclopropyl)phenyl)-6-fluoro-N-(1-(2-hydroxyethyl)-1H-pyrazol-4-yl)quinoline-3-carboxamide; and
4-(4-cyano-4-phenylpiperidin-1-yl)-6-fluoro-N-(1-(2-hydroxyethyl)-1H-pyrazol-4-yl)quinoline-3-carboxamide.

36. The pharmaceutical composition of claim 33, wherein the immunostimulator is at least one selected from the group consisting of an immune checkpoint inhibitor, chimeric antigen receptor (CAR) T-cells, T-cells engineered to express specific TCR targeted tumor antigens (TCR-transgenic), ex vivo expanded T-cells, and bispecific T-cell engagers (BiTE).

37. The pharmaceutical composition of claim 36, wherein at least one of the following applies:
(a) the immunostimulator is an immune checkpoint inhibitor, optionally wherein the immune checkpoint inhibitor is selected from the group consisting of an anti-PD1 antibody, an anti-PD-L1 antibody, an anti-CTLA4 antibody, any fragment thereof, and any combinations thereof;
(b) the immunostimulator is CAR T-cells; and
(c) the at least one immunostimulator comprises an immune checkpoint inhibitor and CAR T-cells.

38. (canceled)

39. (canceled)

40. (canceled)

41. The pharmaceutical composition of claim 33, further comprising at least one selected from the group consisting of a retinoic acid receptor (RAR) inhibitor and a retinoid X receptor (RXR) inhibitor, optionally wherein at least one of the following applies:
(a) the RAR inhibitor is selected from the group consisting of AGN 193109, BMS 195614, BMS 493, CD 2665, ER 50891, LE 135, LY 2955303, MM 11253, any salt or solvate thereof, and any combinations thereof; and
(b) the RXR inhibitor is selected from the group consisting of HX 531, PA 452, and UVI 3003, any salt or solvate thereof, and any combinations thereof.

42. (canceled)

43. (canceled)

44. (canceled)


SEQUENCE LISTING (6 sequences)

SEQ ID NO: 1 - DNA, 20 bp, synthetic construct
  source 1..20: mol_type = other DNA
ctgagttggaccctatatgg

SEQ ID NO: 2 - DNA, 20 bp, synthetic construct
  source 1..20: mol_type = other DNA
gagaatgtgttggtgcctcg

SEQ ID NO: 3 - DNA, 22 bp, synthetic construct
  source 1..22: mol_type = other DNA
caaccctgagcaaatcctccac

SEQ ID NO: 4 - DNA, 25 bp, synthetic construct
  source 1..25: mol_type = other DNA
gacagattgagagcagtgtttaccc

SEQ ID NO: 5 - DNA, 22 bp, synthetic construct
  source 1..22: mol_type = other DNA
tgatatgtcccaggaagatgaa

SEQ ID NO: 6 - DNA, 18 bp, synthetic construct
  source 1..18: mol_type = other DNA
ggaccgagcacttgccta